(19) United States
(12) Patent Application Publication
(10) Pub. No.: US 2024/0400692 A1
(43) Pub. Date: Dec. 5, 2024

(54) CELIAC DISEASE EPITOPES

(71) Applicant: NEXTERA AS, Oslo (NO)

(72) Inventor: Geir Åge LØSET, Oslo (NO)

(21) Appl. No.: 18/695,335

(22) PCT Filed: Sep. 28, 2022
(86) PCT No.: PCT/GB2022/052450

§ 371 Date: Mar. 25, 2024

(30) Foreign Application Priority Data
Sep. 28, 2021 (GB) 2113858.1

(51) Int. Cl. C07K 16/28; C07K 14/74; G01N 33/569

(52) U.S. Cl. CPC C07K 16/2833; C07K 14/70539; G01N 33/56977; C07K 2317/76; C07K 2317/92; G01N 2800/24

(57) ABSTRACT

The present invention provides a peptide comprising an epitope, e.g. a T cell epitope, that comprises the amino acid sequence PYPQQQQPY or an epitope, e.g. a T cell epitope, that comprises the amino acid sequence PYPQQQQPY in which one or more of the Q residues is replaced by an E residue, wherein the peptide is not more than 50 amino acids in length. Conjugates or complexes comprising said peptides and an MHC molecule are also provided, together with various therapeutic and diagnostic uses of said peptides and complexes.

This invention relates generally to the field of epitopes, in particular T cell epitopes. The invention further relates to peptides comprising said epitopes, and products related to or comprising said epitopes or peptides. Such products have therapeutic and diagnostic uses, in particular in the treatment or diagnosis of celiac disease (CeD).

Celiac disease (CeD) is an immune mediated disorder involving immune reactions, in particular abnormal intestinal T cell responses, to dietary gluten proteins from cereals, in particular wheat (gliadin and glutenin), barley (hordein) and rye (secalin).

CeD has a strong genetic basis (genetic predisposition) and hallmarks of the disease are gluten reactive CD4 T cells that recognise deamidated gluten peptides in the context of the HLA-DQ2 (especially HLA-DQ2.5, but also HLA-DQ2.2) or HLA-DQ8 molecules (Lindfors et al., 2019, Nat. Rev. Dis. Primers, 5(3)).

Thus, the disease has a strong HLA association with about 90% of the patients expressing HLA-DQ2.5 (DQA1*05-DQB1*02), and most of the remaining patients expressing HLA-DQ8 (DQA1*03-DQB1*03:02) or HLA-DQ2.2 (DQA1*02:01-DQB1*02). Gluten proteins are resistant to proteolysis due to high proline content and, as a result, long immunogenic peptide fragments remain in the intestine. Currently the T cell response to wheat gluten is thought to be dominated by reactivity to two epitopes of α-gliadin, DQ2.5-glia-α1a (PFPQPELPY (SEQ ID NO:4)) and DQ2.5-glia-α2 (PQPELPYPQ (SEQ ID NO:5)), which can be found within a proteolysis resistant α-gliadin 33mer peptide, as well as to two epitopes of ω-gliadin, DQ2.5-glia-ω1 (PFPQPEQPF (SEQ ID NO:6)) and DQ2.5-glia-ω2 (PQPEQPFPW (SEQ ID NO:7)). Importantly, the immunogenicity of gluten peptides is greatly augmented through post-translational modification by the enzyme transglutaminase 2 (TG2), which by deamidation converts certain glutamine residues (Q) to glutamate (E). The introduction of negatively charged anchor residues presumably makes the peptides better suited for HLA-DQ2.5 binding by increasing the pMHC (peptide-major histocompatibility complex) stability.

Other hallmarks of CeD are antibodies to the autoantigen transglutaminase 2 (TG2) and to gluten peptides (Osman et al., Clin. Exp. Immunol., 2000; 121(2):248-254; Dørum et al., 2016, Sci. Rep. 6, 25565). Both B cells reactive with gluten peptides and B cells reactive with TG2 may serve as antigen presenting cells for gluten reactive (anti-gluten) CD4+ T cells and thereby engage in an amplifying loop for the pathogenic T cell response. Thus, both B cell (antibody) and T cell responses are involved in CeD (Sollid, 2002, supra, Stamnaes and Sollid, Semin. Immuno., 2015; 27(5):343-352).

Although the immune response in CeD is classically observed to proline and glutamine-rich gluten proteins from wheat, barley and rye, some patients also appear sensitive to oat (avenin) as well. CeD primarily affects the small intestine, and classical symptoms include gastrointestinal problems such as chronic diarrhoea, abdominal distension and pain, malabsorption, and loss of appetite. However, as such symptoms are common in other diseases and conditions, diagnosis is often not straightforward. Patients may have severe symptoms and be investigated for a long period of time before a diagnosis of CeD is achieved.

The only established treatment for CeD is a lifelong adherence to a gluten exclusion diet, which generally leads to recovery of the intestinal mucosa, improves symptoms and reduces the risk of developing complications. However, such exclusion diets are incredibly difficult to manage and successfully adhere to, in addition to a growing uncertainty in successful homeostatic re-establishment (Stamnaes et al. Adv Sci (Weinh), volume 8(4), 2021). Thus, flare-ups and relapses in CeD patients are extremely common.

CeD is also difficult to diagnose. Although diagnostic assays based on the detection of TG2 antibodies or anti-gluten peptide antibodies are extremely disease specific, there are issues with such assays (for example low sensitivity or cumbersome procedures) that prevent routine use and mean that many patients suffering from CeD cannot be diagnosed using current methods.

Rates of CeD vary in different parts of the world, from as few as 1 in 300 to as many as 1 in 40, with an average of between 1 in 100 and 1 in 170 people. It is however estimated that 80% of cases remain undiagnosed. CeD can affect children as well as adults.

Improved therapeutic and diagnostic methods for CeD are thus highly desirable. Ideally, an early diagnosis would be possible in people without symptoms, for example, by way of screening.

To this end, over the last few years, work has been done to identify and characterise gluten derived T cell epitopes (gluten derived peptides) which can act to trigger an abnormal T cell response and hence have a potential role in CeD. CD4+ T cells of CeD patients but not healthy subjects recognise gluten peptides when presented by disease associated HLA-DQ molecules (such as DQ2.5, DQ2.2 and DQ8 as discussed above). In addition, it is often observed that gluten reactive T cells of CeD patients recognise the antigenic peptides much better when specific glutamine (Q) residues in the peptides are converted to a glutamate residue (E) by the enzyme TG2. Such converted peptides are also referred to as deamidated peptides or deamidated gluten peptides. CD4+ T cells recognising gluten peptides/epitopes presented by disease-associated HLA-DQ molecules are considered to be drivers of the disease.

Many distinct HLA-DQ restricted T cell epitopes derived from gliadins (α-, γ-, and ω-gliadins), glutenins (both high molecular weight and low molecular weight glutenins), hordeins, secalins and avenins have been identified (Sollid et al., 2020, Immunogenetics 72, 85-88). Such T cell epitopes have a 9 amino acid (9-mer) core region, although most CD4+ T cells recognise peptides longer than 9 amino acids by involvement of N-terminal and C-terminal flanking residues. In addition, gluten specific T cell responses are generally either dependent on, or strongly enhanced by, deamidation of gluten. Although many distinct T cell epitopes have been identified, there is a need to identify others in order to improve therapeutic and diagnostic options for CeD patients. This is particularly the case as it is broadly recognised that the pool of CeD-active gluten epitopes recognised by CD4+ T cells is far from complete, as the epitopes recognised by many T cells are not known (Sherf et al., 17 Mar. 2020, Front.Nutr.).

The present invention is based on the identification of a new T cell epitope which is associated with CeD. Surprisingly, this epitope has not to date been found in classical hexaploid bread wheat (Triticum aestivum), but is found in a diploid wild type of wheat (Tritcum urartu). Notably, the size and complexity of the wheat genomes, as well as the lack of genome-assembly data for multiple wheat lines are likely cause for masking CeD relevant epitopes in hexaploid bread wheat, including the new epitope disclosed herein (Walkowiak et al, Nature, 2020, 588(7837):277). This, may well underlie the prevailing lack of known epitope reactivities in CeD (Raki et al Gastroenterology, 2017, 153(3):787). Such epitopes thus provide an exciting new opportunity for CeD therapy and diagnosis.

This epitope was not identified using conventional techniques but was identified due to a surprising finding that an antibody selected for its ability to interact with the immunodominant α-gliadin epitope, DQ2.5-glia-α1a, showed some properties, which upon further investigation suggested that the antibody might also be recognising another T cell epitope in CeD patients. Through a mixture of extensive database analysis and in vitro testing it has now been shown that this antibody can recognise a new T cell epitope associated with CeD. This T cell epitope has a 9-mer core sequence (non-deamidated sequence) PYPQQQQPY (SEQ ID NO:8). Deamidated forms thereof are also provided.

Thus, in one aspect, the present invention provides a peptide, e.g. an isolated peptide, that comprises an epitope that comprises (or consists of) the amino acid sequence PYPQQQQPY (SEQ ID NO:8), or an epitope that comprises (or consists of) the amino acid sequence PYPQQQQPY (SEQ ID NO:8) in which one or more of the Q residues is replaced by an E residue.

Viewed alternatively, the present invention provides a peptide, e.g. an isolated peptide, that comprises the amino acid sequence PYPQQQQPY (SEQ ID NO:8), or that comprises the amino acid sequence PYPQQQQPY (SEQ ID NO:8) in which one or more of the Q residues is replaced by an E residue.

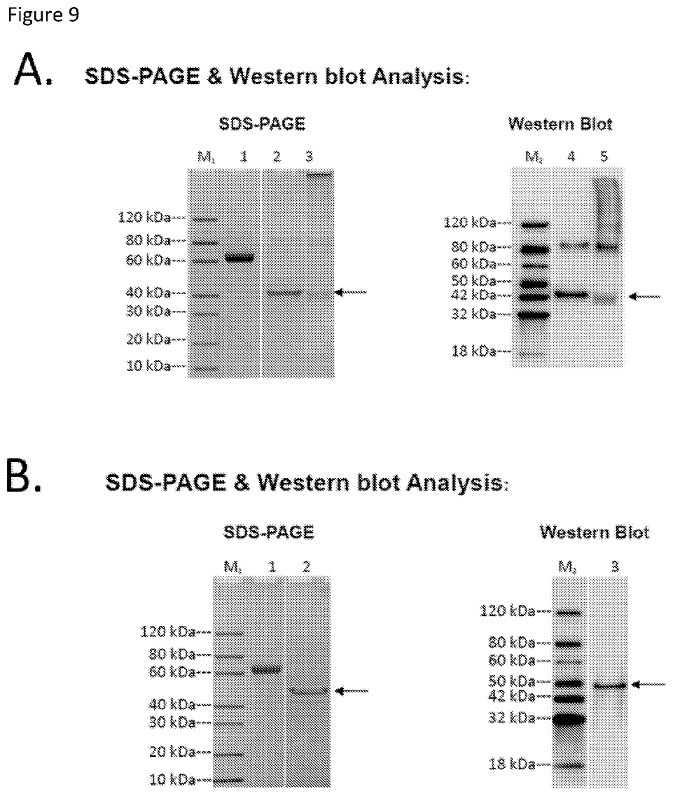

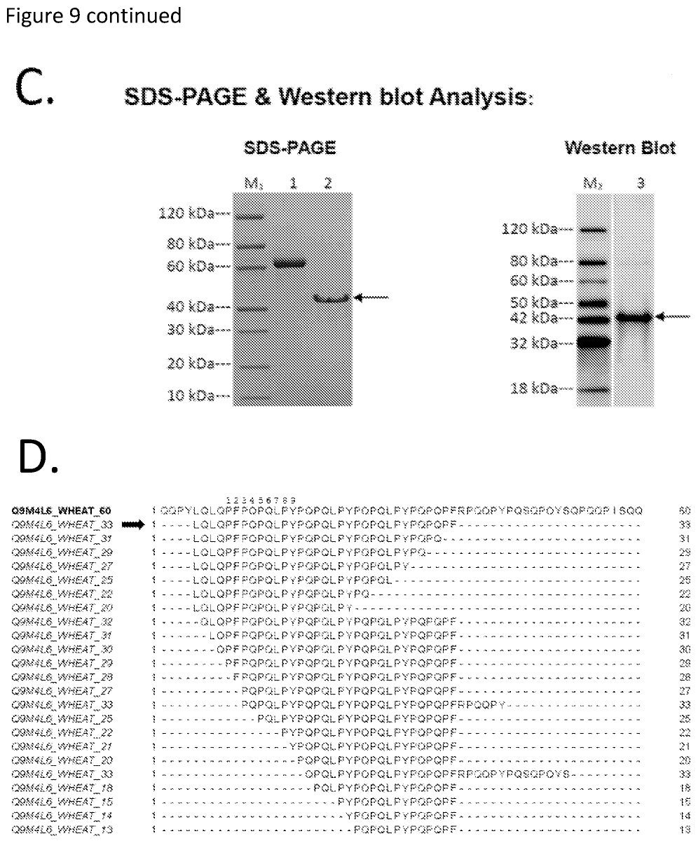

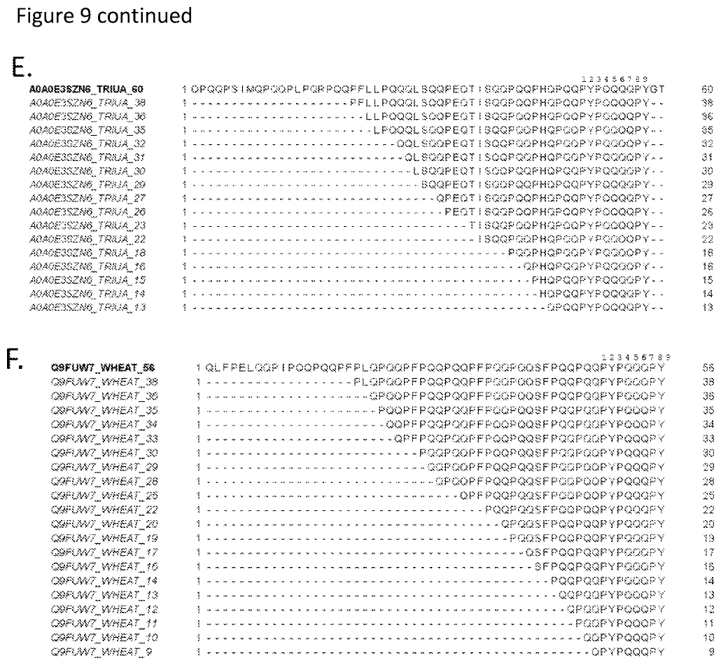

Peptides of the invention thus comprise the above 9 amino acid sequence (9-mer) which can be regarded as the core sequence or core epitope sequence. However, when such core sequences are associated with MHC molecules, i.e. where peptide-MHC (p-MHC) complexes are concerned, then flanking residues at the N- and/or C-terminus of the 9-mer are also typically important for the interaction. Thus, peptides with longer sequences are also contemplated, for example, peptides with the above 9-mer and with additional amino acids at the N- and/or C-terminus. Any appropriate number of additional flanking amino acid residues can be included. For example, such additional residues can be included provided that the peptide has the ability to associate with or form complexes with or bind to an MHC molecule, e.g. HLA-DQ2.5 or HLA-DQ2.2. Preferred examples include sequences with up to 4, e.g. 1, 2, 3 or 4 amino acid residues at either the N-terminus and/or the C-terminus of the above 9-mer. Other preferred examples include sequences which terminate with the Y or PY residues at position 9, and positions 8 and 9, respectively, of the above 9-mer, i.e. which do not have any flanking residues at the C-terminus. Some exemplary sequences are shown in FIG. 9E.

The two residues immediately flanking the 9-mer, i.e. position−1 or 10 (when the 9 amino acids of the 9-mer are referred to as positions 1 to 9 from the N-terminus to the C-terminus), can be particularly important for the interaction of a peptide with an MHC molecule, e.g. the interaction of a peptide with the peptide binding groove of an MHC molecule. Thus, preferred peptides of the invention may comprise a Q residue at position−1 and/or a G residue at position 10, e.g. comprise the amino acid sequence QPYPQQQQPY (SEQ ID NO:9), PYPQQQQPYG (SEQ ID NO:10) or QPYPQQQQPYG (SEQ ID NO:11), or a deamidated version thereof. Although any one or more of the Q residues can be replaced with an E residue in such deamidated versions or forms of the peptides, preferred and convenient positions for deamidation (or positioning of E residues) are shown underlined.

Other preferred peptides of the invention may comprise QQ at the N-terminus of the 9-mer and/or GT at the C-terminus of the 9-mer. For example, such peptides comprise the amino acid sequence QQPYPQQQQPY (SEQ ID NO:12), PYPQQQQPYGT (SEQ ID NO:13), or QQPYPQQQQPYGT (SEQ ID NO:14), or a deamidated version thereof. In some embodiments, peptides comprising QQPYPQQQQPY (SEQ ID NO:12), or QQPYPQQQQPYG (SEQ ID NO:15), or a deamidated version thereof, are preferred. Although any one or more of the Q residues can be replaced with an E residue in such deamidated versions or forms of the peptides, preferred and convenient positions for deamidation (or positioning of E residues) are shown underlined. In exemplary peptides, one or both of these positions may be deamidated (or otherwise provided) to replace the Q residue with an E residue.

Other preferred peptides of the invention may comprise PQQ at the N-terminus of the 9-mer and/or GTS at the C-terminus of the 9-mer. For example, such peptides comprise the amino acid sequence PQQPYPQQQQPY (SEQ ID NO:16), PYPQQQQPYGTS (SEQ ID NO:17), or PQQPYPQQQQPYGTS (SEQ ID NO:18), or a deamidated version thereof. In some embodiments, peptides comprising PQQPYPQQQQPY (SEQ ID NO:16), or PQQPYPQQQQPYG (SEQ ID NO:19), or a deamidated version thereof, are preferred.

Although any one or more of the Q residues can be replaced with an E residue in such deamidated versions or forms of the peptides, preferred and convenient positions for deamidation (or positioning of E residues) are shown underlined. In exemplary peptides, one or both of these positions may be deamidated (or otherwise provided) to replace the Q residue with an E residue.

Other preferred peptides of the invention may comprise QPQQ at the N-terminus of the 9-mer and/or GTSL at the C-terminus of the 9-mer. For example, such peptides comprise the amino acid sequence QPQQPYPQQQQPY (SEQ ID NO:20), PYPQQQQPYGTSL (SEQ ID NO:21) or QPQQPYPQQQQPYGTSL (SEQ ID NO:22), or a deamidated version thereof. In some embodiments, peptides comprising QPQQPYPQQQQPY (SEQ ID NO:20), QPQQPYPQQQQPYG (SEQ ID NO:23), or a deamidated version thereof, are preferred. Although any one or more of the Q residues can be replaced with an E residue in such deamidated versions or forms of the peptides, preferred and convenient positions for deamidation (or positioning of E residues) are shown underlined. In exemplary peptides, one or both of these positions may be deamidated (or otherwise provided) to replace the Q residue with an E residue.

Combinations of the above examples of 1, 2, 3 or 4 flanking residues are also provided. Thus, for example, 1 flanking residue at the N-terminus or the C-terminus can be combined with 2, 3 or 4 of the above described flanking residues at the other terminus, or 2 flanking residues at the N-terminus or the C-terminus can be combined with 1, 3 or 4 of the above described flanking residues at the other terminus, or 3 flanking residues at the N-terminus or the C-terminus can be combined with 1, 2 or 4 of the above described flanking residues at the other terminus, or 4 flanking residues at the N-terminus or the C-terminus can be combined with 1, 2 or 3 of the above described flanking residues at the other terminus.

Other exemplary peptides of the invention only comprise additional amino acids at the N-terminus of the 9-mer.

Peptides of the invention are generally up to 50 amino acids in length (or no longer than 50 amino acids in length). Preferred peptides are thus up to (or no longer than) 50, 45, 40, 38, 36, 35, 34, 33, 32, 31, 30, 29, 28, 27, 26, 25, 24, 23, 22, 21, 20, 19, 18, 17, 16, 15, 14, 13, 12, 11, 10 or 9 amino acids in length, or are these lengths. In this aspect of the invention, the minimal length of the peptides is 9 amino acids so that the core 9-mer epitope sequence, e.g. core T cell epitope sequence, as outlined above, or deamidated forms thereof, is incorporated. Thus, preferred peptides are, or are at least, 9, 10, 11, 12, 13, 14, 15, 16, 17, 18, 19 or 20 amino acids in length. For example, from 9 to 50, 45, 40, 35, 30 or 25 amino acids in length, e.g. from 10, 11, 12, 13, 14, 15, 16 or 17 to 50, 45, 40, 35, 30, 25 or 20 amino acids in length. Other preferred peptides are up to 15 amino acids in length, for example, 9 to 15 amino acids in length, for example, 13 amino acids in length. Other preferred peptides are up to 20 amino acids in length, for example, 9 to 20 amino acids in length, for example, 17 amino acids in length. Other preferred peptides are up to 25 or 30 amino acids in length, for example, 9 to 25 or 9 to 30 amino acids in length, for example, 13 or 17 amino acids in length. Other preferred peptides are up to 40 amino acids in length, for example, 9 to 40 or 9 to 38 amino acids in length, for example, are the lengths as shown in FIG. 9E, which also shows the sequences of some exemplary peptides of the invention. Deamidated (or E residue containing) versions thereof are sometimes preferred.

Thus, embodiments of the invention provide a peptide, e.g. an isolated peptide, that comprises an epitope that comprises (or consists of) the amino acid sequence PYPQQQQPY (SEQ ID NO:8), or an epitope that comprises (or consists of) the amino acid sequence PYPQQQQPY (SEQ ID NO:8) in which one or more of the Q residues is replaced by an E residue, wherein the peptide is not more than 50 amino acids in length, with preferred lengths and preferred and exemplary sequences as described above and elsewhere herein.

Peptides or isolated peptides in accordance with the present invention of course do not include the full-length omega gliadin protein from Triticum urartu in which the peptides and epitopes of the invention can be found (i.e. wild-type or naturally occurring omega gliadin Triticum urartu protein), or any other full-length (wild-type or naturally occurring) gliadin protein, or any other full-length (wild-type or naturally occurring) protein in the omega gliadin family, or any other full-length (wild-type or naturally occurring) proteins. Peptides or isolated peptides in accordance with the present invention thus do not include full-length SEQ ID NO:1 (which corresponds to entry number A0A0E3SZN6 in the Uniprot database (https://www.uniprot.org/uniprotA0A0E3SZN6) or Uniparc accession number: UPI000618D06A.

Given their proposed association with CeD, peptides or isolated peptides of the present invention can be found in or can be derived from a gluten protein, or a gliadin protein. Such sequences are therefore generally naturally occurring sequences, e.g. fragments of naturally occurring sequences or deamidated versions thereof.

Peptides or isolated peptides of the present invention, thus, for example, correspond to (or correspond essentially to) regions or fragments (or epitopes) of gluten or gliadin proteins, e.g. regions or fragments (or epitopes) of whole or full-length gluten or gliadin proteins, e.g. of the full-length (wild type or naturally occurring) omega gliadin Triticum urartu protein (e.g. as described elsewhere herein) or the equivalent or similar sequence in an alternative gluten or gliadin protein. Some peptides of the invention are believed to themselves occur in nature (i.e. they do have naturally occurring counterparts or occur in nature, e.g. are naturally occurring fragments). Some peptides of the invention do not however occur per se in nature (i.e. they do not have naturally occurring counterparts or do not occur in nature, e.g. are not naturally occurring fragments). Thus, some peptides of the invention (or complexes or conjugates comprising said peptides) can be considered to be artificial peptides, or synthetic peptides, or man-made peptides, or non-native peptides.

By “corresponds to” in this context is meant that the amino sequence (SEQ ID NO:) of the isolated peptide matches the amino acid sequence of the equivalent region or epitope of the wild type (or naturally occurring) omega gliadin Triticum urartu protein (SEQ ID NO:1). By “corresponds essentially to” is meant that the amino acid sequence of the peptide (SEQ ID NO:) is identifiable as being based on (or derived from or a modified form of) the sequence of the equivalent region or epitope of the wild type (or naturally occurring) omega gliadin Triticum urartu protein (SEQ ID NO:1). For example, a peptide having a sequence that “corresponds essentially to” the equivalent region or epitope of the wild type (or naturally occurring) omega gliadin Triticum urartu protein, typically has one or more (e.g. 1, 2, 3, 4 or 5, preferably 1, 2 or 3) amino acid substitutions, additions or deletions as compared to a peptide that corresponds to (i.e. exactly corresponds to) the sequence of the equivalent region or epitope of the wild type (or naturally occurring) omega gliadin Triticum urartu protein. Thus, a peptide having a sequence that “corresponds essentially to” the equivalent region or epitope of the wild type (or naturally occurring) omega gliadin Triticum urartu protein may be considered to be a “substantially homologous” peptide sequence as defined elsewhere herein.

Preferred peptides of the invention comprise the amino acid sequence QPQQPYPQQQQPY (SEQ ID NO:20) (13-mer), PQQPYPQQQQPYGT (SEQ ID NO:24) (14-mer), or QPQQPYPQQQQPYGTSL (SEQ ID NO:22) (17-mer), or said sequence wherein one or more of said Q residues is replaced by an E residue.

As mentioned elsewhere herein, peptides or epitopes associated with CeD generally contain one or more residues in which a Q has been changed to an E residue. Such E containing sequences are thus preferred in some aspects and are also referred to herein as deamidated peptides, deamidated versions, or deamidated forms, or other similar or equivalent terminology. Peptide sequences in which one or more, or all, Q residues are replaced by E residues (or where E residues are present instead of Q residues) are thus contemplated by the present invention. Thus, with reference to the 9-mer core sequence described herein (PYPQQQQPY (SEQ ID NO:8)), 1, 2, 3 or 4 of the Q residues at (or corresponding to) positions 4, 5, 6 or 7 of the 9-mer can be replaced by E residues. However, with reference to the 9-mer core sequence, preferred peptides of the invention have the Q residue at position 6 replaced by an E residue. Other peptides of the invention have the Q residue at position 4 replaced by an E residue. Other peptides of the invention have the Q residue at position 6 and the Q residue at position 4 replaced by an E residue. If the 9-mer is present in a longer peptide then the appropriate positions of the E residues are adjusted accordingly depending on where the 9-mer is present in the sequence. In other words, these positions (and other amino acid positions as described herein, unless indicated otherwise) are defined in relation to the 9-mer core sequence and longer peptide sequences have E residues at positions corresponding to the above-described positions in the 9-mer (e.g. at position 6 and/or position 4, or positions corresponding to position 6 and/or position 4).

Thus, preferred peptides of the invention comprise the sequence PYPQQEQPY (SEQ ID NO:25), PYPEQEQPY (SEQ ID NO:26), or PYPEQQQPY (SEQ ID NO:27).

As described elsewhere herein, in CeD patients the transglutaminase-2 (TG2) enzyme is believed to be responsible for physiological deamidation of gluten derived sequences observed in the development of CeD. Thus, peptides comprising sequences which have been deamidated by the TG2 enzyme or peptides comprising sequences obtainable by TG2 deamidation or peptides comprising sequences that correspond to sequences which have been deamidated by the TG2 enzyme are preferred. These sequences thus represent exemplary and preferred deamidated peptides of the invention. The TG2 enzyme uses QXP residues as a substrate and can convert this sequence to EXP via a deamidation reaction (Sollid, 2002, Nature Reviews Immunology 2:647-655). Thus, in the above described non-deamidated (healthy or wildtype) 9-mer PYPQQQQPY (SEQ ID NO:8), the Q residue at position 6 is the most likely residue to be deamidated in CeD patients. Thus, peptides which comprise the sequence PYPQQEQPY (SEQ ID NO:25) are particularly preferred. Specific examples of such peptides are peptides which comprise QPQQPYPQQEQPY (SEQ ID NO:28), PQQPYPQQEQPYGT (SEQ ID NO:29), or QPQQPYPQQEQPYGTSL (SEQ ID NO:30).

In these longer peptides, QPQQPYPQQEQPY (SEQ ID NO:28), PQQPYPQQEQPYGT (SEQ ID NO:29), or QPQQPYPQQEQPYGTSL (SEQ ID NO:30), and in some of the other longer peptides of the invention, there is a further classical substrate (QXP sequence/motif) for the TG2 enzyme, i.e. here the sequence QQP which can be deamidated to EQP. This motif is shown in the sequences above where the Q that can be deamidated to an E residue is shown underlined. Again, peptides with an E residue at this position are preferred in some aspects.

In other embodiments, peptides of the present invention comprise the sequence QPQQPYPQQQQPY (SEQ ID NO:20), wherein said Q at residue 3 is replaced by an E residue, and/or wherein said Q at residue 10 (corresponding to position 6 in the core 9-mer of the epitope of the invention, e.g. the T cell epitope) is replaced by an E residue. Optionally, or in addition, said Q at residue 8 (corresponding to position 4 in the core 9-mer of the epitope of the invention, e.g. the T cell epitope) is replaced by an E residue.

Thus, in some embodiments, peptides of the present invention comprise the sequence PQQPYPQQQQPYGT (SEQ ID NO:24), wherein said Q at residue 2 is replaced by an E residue, and/or wherein said Q at residue 9 (corresponding to position 6 in the core 9-mer of the epitope of the invention, e.g. the T cell epitope) is replaced by an E residue. Optionally, or in addition, said Q at residue 7 (corresponding to position 4 in the core 9-mer of the epitope of the invention, e.g. the T cell epitope) is replaced by an E residue.

In other embodiments, peptides of the present invention comprise the sequence QPQQPYPQQQQPYGTSL (SEQ ID NO:22), wherein said Q at residue 3 is replaced by an E residue, and/or wherein said Q at residue 10 (corresponding to position 6 in the core 9-mer of the epitope of the invention, e.g. the T cell epitope) is replaced by an E residue. Optionally, or in addition, said Q at residue 8 (corresponding to position 4 in the core 9-mer of the epitope of the invention, e.g. the T cell epitope) is replaced by an E residue.

Preferred examples of such embodiments, are peptides which comprise the sequence;

(SEQ ID NO: 28)
QPQQPYPQQEQPY,
(SEQ ID NO: 31)
QPEQPYPQQEQPY,
(SEQ ID NO: 32)
QPEQPYPQQQQPY,
(SEQ ID NO: 30)
QPQQPYPQQEQPYGTSL,
(SEQ ID NO: 33)
QPEQPYPQQEQPYGTSL,
(SEQ ID NO: 34)
QPEQPYPQQQQPYGTSL,
(SEQ ID NO: 29)
PQQPYPQQEQPYGT,
(SEQ ID NO: 35)
PEQPYPQQEQPYGT,
or
(SEQ ID NO: 36)
PEQPYPQQQQPYGT.

In these peptides the second underlined residue corresponds to position 6 in the core 9-mer of the epitope of the invention, e.g. the T cell epitope 9-mer.

Other specific examples might include the above peptides with the inclusion of an additional E residue at the position corresponding to position 4 in the core 9-mer of the epitope of the invention, e.g. the T cell epitope 9-mer, or include the above peptides where the only E residue is at the position corresponding to position 4 in the core 9-mer of the epitope of the invention, e.g. the T cell epitope 9-mer (in other words peptides in which both the underlined residues in the above 9 listed peptides are Q residues and the position corresponding to position 4 in the core 9-mer of the epitope of the invention, e.g. the T cell epitope 9-mer, is an E residue.

As mentioned elsewhere herein, CeD is believed to involve both B cell and T cell responses. Peptides comprising the core 9-mer of the newly identified epitope, for example, T cell epitope, of the invention are described herein. However, some of the peptides described above and elsewhere herein also contain a second epitope, QPQQPYP (SEQ ID NO:37), which can for example, act as a B cell epitope. Peptides comprising or further comprising such epitopes, or deamidated versions of such epitopes (or versions where E residues are present in place of Q residues) in which one or more of the Q residues, e.g. 1, 2 or 3 of the Q residues at positions 1, 3 or 4 of this B cell epitope (or at positions corresponding to these positions), is replaced by an E residue, are preferred.

The Q residue at position 3 of this epitope, e.g. B cell epitope, sequence, QPQQPYP (SEQ ID NO:37), is the most likely substrate for the TG2 enzyme (it is in the motif QQP which is a classic QXP motif for the TG2 enzyme and should be converted to EQP). Thus, due to the proposed role of TG2 in the development of CeD, particularly preferred second epitope or B cell epitope sequences found in the peptides of the present invention comprise the sequence QPEQPYP (SEQ ID NO:38).

An alternative aspect of the invention thus provides an epitope, e.g. a B cell epitope, with the sequence QPQQPYP (SEQ ID NO:37) or deamidated versions thereof (or versions where E residues are present in place of Q residues) as described above, in particular QPEQPYP (SEQ ID NO:38). Peptide sequences comprising this epitope, e.g. B cell epitope sequence, QPQQPYP (SEQ ID NO:37), or deamidated versions thereof as described above, in particular QPEQPYP (SEQ ID NO:38), provide a yet further aspect.

Some peptides of the invention comprise both a first or core 9-mer comprising epitope, e.g. a T cell epitope, and a second epitope, e.g. a B cell epitope, for example, as defined herein. Preferred such peptides have an overlapping first epitope, e.g. a T cell epitope, and second epitope, e.g. B cell epitope. In other words, in such embodiments at least one of the amino acid residues of the first epitope, or T cell epitope, also forms part of the second epitope or B cell epitope, and vice versa. For example, in particularly preferred peptides of the invention, the PYP residues at the end of the second (or B cell) epitope also form part of the first (or T cell) epitope, e.g. provide the first three amino acids of the first (or T cell) epitope. Preferred examples of such embodiments are peptides which comprise the sequence;

(SEQ ID NO: 28)
QPQQPYPQQEQPY,
(SEQ ID NO: 31)
QPEQPYPQQEQPY,
(SEQ ID NO: 32)
QPEQPYPQQQQPY,
(SEQ ID NO: 30)
QPQQPYPQQEQPYGTSL,
(SEQ ID NO: 33)
QPEQPYPQQEQPYGTSL,
or
(SEQ ID NO: 34)
QPEQPYPQQQQPYGTSL.

Particularly preferred peptides in accordance with this embodiment comprise the sequence QPEQPYPQQEQPY (SEQ ID NO:31) or QPEQPYPQQEQPYGTSL (SEQ ID NO:33). Other examples comprise the sequence QPEQPYPQQEQPYG (SEQ ID NO:39), or versions thereof with Q residues in place of one or both E residues.

When ingested, dietary glutens are broken down by proteases in the intestinal space, e.g. in the lumen of the small intestine, to form partially digested gluten peptides or proteolysis resistant peptides. Deamidation of these peptides by TG2, for example, in the lamina propria of the small intestine, can then occur as part of the mechanism of development of CeD. Recognition of such deamidated peptides by T cells and B cells is believed to be involved in CeD. Thus, in some embodiments of the invention, preferred peptides contain or comprise or consist of or correspond to such naturally occurring (native) peptides, e.g. naturally occurring partially digested gluten peptides or naturally occurring proteolysis resistant peptides (or deamidated versions thereof, in particular naturally occurring deamidated versions thereof). Tryptic digest analysis of the peptides of the invention has shown that the peptide sequences QPQQPYPQQQQPY (SEQ ID NO:20) and/or QPQQPYPQQQQPYGTSL (SEQ ID NO:22), or naturally occurring variants thereof, e.g. substantially homologous sequences that, for example, have Y or PY (as shown at the end of the first sequence) as the final (or C-terminal) amino acids in a substantially homologous sequence, or L or SL (as shown at the end of the second sequence) as the final (or C-terminal) amino acids in a substantially homologous sequence, are the most likely to occur (or be produced) naturally in the small intestine. Thus, in some embodiments these sequences, or sequences comprising (or consisting of) these sequences, or sequences produced (e.g. in vitro) or predicted (e.g. in silico) by tryptic digest, e.g. trypsin and/or chymotrypsin digest, or naturally occurring variants thereof (or deamidated versions thereof, as described elsewhere herein) are preferred. Exemplary peptide sequences are shown in FIG. 9E and peptides with these sequences or comprising (or consisting of) these sequences are exemplary sequences, in particular peptides ending (or terminating) with the residues PY or Y, e.g. as described in FIG. 9E or elsewhere herein. In particular, deamidated (or E residue containing) versions of such peptides are provided, preferably with E residues at the positions described elsewhere herein, e.g. at one or more of positions 6, −2 and 4, e.g. position 6 and/or −2, in relation to the 9-mer core sequence as shown in FIG. 9E.

Preferred peptides or epitopes of the invention are isolated peptides or epitopes. Preferred epitopes of the invention are T cell epitopes or B cell epitopes, in particular T cell epitopes. Thus, preferred peptides of the invention comprise or contain or consist of or correspond to sequences that are T cell epitopes or B cell epitopes. Other preferred peptides of the invention comprise or contain or consist of or correspond to sequences that contain T cell epitopes and B cell epitopes.

The term “T cell epitope” as used herein refers to an amino acid sequence which can bind to, be associated with, form a complex with, or be presented in an antigenic peptide groove of an appropriate MHC/HLA molecule, here an HLA-DQ 2.5 or HLA-DQ 2.2 molecule. Such T cell epitopes can also, when associated with said HLA molecules, be recognized or bound by a T cell receptor, and thereby activate T cells or promote T cell reactivity, e.g. stimulate proliferation of T cells. These T cells are also sometimes referred to herein as gluten specific (or gluten reactive) T cells or gluten specific (or gluten reactive) CD4+ T cells. Nine amino acids is the typical length of the core region of a T cell epitope.

The term “B cell epitope” as used herein refers to an amino acid sequence that can be recognised by or bound by antibody molecules (or B cell receptors). Such antibody molecules can be present either on B cells (as B cell receptors) or in a soluble form. The length of peptide sequence that is recognized by or bound by an antibody, e.g. the length of a B cell epitope, is highly variable. As described above, the specific B cell epitope described herein comprises 7 amino acids but B cell epitopes can be shorter or longer than this.

The peptides or epitopes of the invention, e.g. T cell epitopes, can be classified as gliadin-omega (ω) peptides or epitopes. The peptides or epitopes of the invention, e.g. T cell epitopes, are preferably DQ2.5 restricted. In other words, they are peptides or epitopes which can be bound to or associated with or presented by the MHC class II/HLA molecule HLA-DQ2.5. Alternatively, or additionally, the peptides or epitopes of the invention, e.g. T cell epitopes, can be DQ2.2 restricted. In other words, they are peptides or epitopes which can be bound to or associated with or presented by the MHC class II/HLA molecule HLA-DQ2.2. Thus peptides or epitopes of the invention are capable of forming complexes (pMHC complexes) with the MHC class II/HLA molecule HLA-DQ2.5 or HLA-DQ2.2, preferably HLA-DQ2.5.

HLA-DQ2.5 (encoded by DQA1*05 and DQB1*02) is a specific type of MHC Class II molecule that has a strong association with CeD. HLA-DQ2.2 (encoded by DQA1*02:01-DQB1*02) is another specific type of MHC Class II molecule that has an association with CeD.

HLA-DQ2.5 comprises an α-chain (typically having an α₁ domain and an α₂ domain and typically encoded by DQA1*05) and a β-chain (typically having a β₁ domain and a β₂ domain and typically encoded by DQB1*02). Amino acid sequences of the α- and β-chains of HLA-DQ2.5 are set forth herein (the α-chain sequence is set forth in SEQ ID NO:2; the β-chain sequence is set forth in SEQ ID NO:3).

HLA-DQ2.5 (or HLA-DQ2.2) can present gliadin peptides or epitopes, for example, peptides or epitopes of α-gliadin or ω-gliadin, to T cells (e.g. CD4+ T cells). Preferred peptides or epitopes presented by HLA-DQ2.5 (or HLA-DQ2.2) are the peptides or epitopes, e.g. the T cell epitopes, of the present invention as described herein. The amino acid sequence of various deamidated DQ2.5 epitopes and peptides of the present invention are provided elsewhere herein. These deamidated forms of the DQ2.5 epitopes and peptides of the invention may be considered to be CeD-associated forms of DQ2.5 epitopes and peptides and are generally preferred.

Although the longer peptides of the invention, for example, the peptides such as QPQQPYPQQQQPY (SEQ ID NO:20), PQQPYPQQQQPYGT (SEQ ID NO:24), or QPQQPYPQQQQPYGTSL (SEQ ID NO:22) (or deamidated versions thereof, as described herein) may associate with (or bind to) HLA-DQ2.5 (or HLA-DQ2.2), the binding groove (or binding pocket or peptide groove, or antigenic peptide groove) of the HLA-DQ2.5 (or HLA-DQ2.2) molecule (i.e. the MHC molecule) can only present, or accommodate, a single 9-mer epitope (e.g. the DQ2.5-core epitope PYPQQQQPY (SEQ ID NO:8), or a deamidated equivalent sequence, as described herein) at a given time. Which epitope is presented by HLA-DQ2.5 (or HLA-DQ2.2) is determined by the “register” (or position) in which the longer peptide is bound to (associated with) the HLA-DQ2.5 (or HLA-DQ2.2).

The non-disease associated forms of the epitopes or peptides, e.g. the DQ2.5-epitopes or peptides, of the invention (or “native” form or non-deamidated form or “healthy” form) comprise the sequence PYPQQQQPY (SEQ ID NO:8).

A preferred disease associated form of the epitopes or peptides, e.g. the DQ2.5 epitopes or peptides, of the invention (deamidated form or CeD-associated form or pathogenic form) comprise the sequence PYPQQEQPY (SEQ ID NO:25).

The non-disease associated forms of the epitopes may also be present on a longer peptide, e.g. a proteolysis resistant longer peptide, which comprises (or consists of) the sequence QPQQPYPQQQQPY (SEQ ID NO:20) or QPQQPYPQQQQPYGTSL (SEQ ID NO:22), or a naturally occurring variant thereof.

Similarly, the disease associated forms of the epitopes may also be present on a longer peptide, e.g. a proteolysis resistant longer peptide, and can comprise (or consist of) deamidated forms of the sequence QPQQPYPQQQQPY (SEQ ID NO:20) or QPQQPYPQQQQPYGTSL (SEQ ID NO:22), or a naturally occurring variant thereof, for example,

(SEQ ID NO: 28)
QPQQPYPQQEQPY,
(SEQ ID NO: 31)
QPEQPYPQQEQPY,
(SEQ ID NO: 32)
QPEQPYPQQQQPY,
(SEQ ID NO: 30)
QPQQPYPQQEQPYGTSL,
(SEQ ID NO: 33)
QPEQPYPQQEQPYGTSL,
(SEQ ID NO: 34)
QPEQPYPQQQQPYGTSL,

or other E residue containing peptides as described elsewhere herein, or a naturally occurring variant of any of the above.

Peptides or epitopes which comprise sequences that are substantially homologous to any of the sequences provided herein are also provided.

In the context of the peptide sequences of the invention, a sequence “substantially homologous” to a given amino acid sequence may be a sequence having, or a sequence comprising, a sequence containing 1, 2, 3, 4, 5 or 6 (preferably 1, 2 or 3) amino acid substitutions or deletions or additions compared to the given amino acid sequence, or a sequence having at least 60%, 70%, 80%, 85%, 88%, 90%, 92%, 94%, 95%, 96%, 97%, 98% or 99% sequence identity to the given amino acid sequence, or a sequence having at least 6 consecutive amino acids of the given amino acid sequence.

In some preferred embodiments, amino acid sequences that are “substantially homologous” to peptides of the invention are sequences having, or sequences comprising, a sequence that has 1, 2, or 3 amino acid substitutions or additions or deletions (preferably 1 or 2, more preferably 1) compared with the amino acid sequence of the given peptide.

Amino acid sequences that are “substantially homologous” to peptides of the invention include sequences that comprise (or consist of) at least 6 consecutive amino acids of the isolated peptides (or comprise or consist of at least 7, at least 8, at least 9, at least 10, at least 11, at least 12, at least 15, at least 20, at least 25 or at least 30 consecutive amino acids of the isolated peptide). Nine amino acids is the typical length of the core region of a T cell epitope that is presented by an appropriate HLA molecule and that is recognized or bound by a T cell receptor. Six or seven amino acids, or longer, might be a typical length of peptide/protein sequence that is recognized or bound by an antibody, e.g. is a typical length of a B cell epitope. As described elsewhere herein peptides comprising T cell and/or B cell epitopes, e.g. comprising the sequences PYPQQQQPY (SEQ ID NO:8) and/or QPQQPYP (SEQ ID NO:37), or deamidated versions thereof (if both are present then these sequences can overlap), are preferred. Thus, other exemplary peptides of the invention comprise sequences which are substantially homologous to sequences comprising the T cell and/or B cell epitopes described herein.

Alternative peptides or substantially homologous peptides of the invention include sequences that are longer than 9 amino acids and which comprise the 9-mer core sequence PYPQQQQPY (SEQ ID NO:8). In other words, preferred substantially homologous peptides have peptide sequences as defined herein but wherein the amino acid variation, e.g. the amino acid substitutions, deletions or additions as described herein (e.g. the 1, 2 or 3 changes, preferably 1 or 2, more preferably 1), are located outside of the 9-mer core sequence. Appropriate lengths for such peptides are described elsewhere herein.

Alterations in the amino acid sequences can be with conservative or non-conservative amino acids. Preferably said alterations are amino acid substitutions. Preferably said alterations are conservative amino acid substitutions.

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine).

The term “substantially homologous” also includes modifications or chemical equivalents of the amino acid sequences of the present invention that perform substantially the same function as the amino acid sequences of the invention in substantially the same way. For example, any substantially homologous peptide encompassed by the invention should typically retain the ability to act as a T cell epitope and/or B cell epitope, as appropriate. In the case of a T cell epitope, such substantially homologous peptides, e.g. retain the ability to bind to or associate with an MHC class II/HLA molecule, in particular HLA-DQ2, for example, HLA-DQ2.5 (or HLA-DQ2.2).

Alternatively, or in addition (preferably in addition), such substantially homologous peptides have the ability to bind to or interact with or be recognised by a T cell receptor (conveniently a TCR which binds to or recognises the unmodified or non-variant sequence) and/or to activate or stimulate a T cell response, e.g. T cell proliferation or inflammatory cytokine production.

In the case of a B cell epitope, such substantially homologous peptides, e.g. retain the ability to be bound by anti-gluten or anti-gliadin (e.g. anti-T. urartu omega gliadin) antibodies or B cell receptors, in particular antibodies (or BCRs) which can bind to or recognise peptides (e.g. unmodified or non-variant peptides) of the present invention.

Alternatively, or in addition, such substantially homologous peptides retain the ability to act as a peptide or epitope to (or against) which antibodies which bind to omega gliadin, e.g. the omega gliadin of T. urartu, can be generated (or raised).

The above functional properties of substantially homologous peptides of the invention can conveniently be assessed by comparison to an unmodified or wildtype or parent peptide sequence of the invention. Typically, such functional properties of substantially homologous peptides will be observed to at least the same amount, level or extent as observed with (or when compared to) the unmodified, wildtype or parent peptide sequence.

Methods of carrying out the above described manipulation of amino acids (e.g. to generate “substantially homologous” sequences) are well known to a person skilled in the art.

Preferably such peptides or substantially homologous peptides of the invention retain a Q or preferably an E residue at the position corresponding to position 6 of the core 9-mer as described herein.

Other preferred peptides or substantially homologous peptides of the invention comprise a G residue at position 10, or at the position corresponding to position 10 in relation to the core 9-mer as described herein. Exemplary such sequences will comprise a G residue at position 10, or GT residues at positions 10 and 11, respectively, or G and L residues at positions 10 and 13, respectively, or GTSL residues at positions 10, 11, 12 and 13 respectively (or corresponding positions).

Other preferred peptides or substantially homologous peptides of the invention comprise naturally occurring peptides (or sequences substantially homologous thereto). In particular, preferred peptides of the invention are those which correspond to peptides found in the small intestine, for example, naturally occurring proteolytic fragments (or proteolysis resistant peptides) that are obtained or obtainable by the action of proteases in the gastrointestinal tract. The action of such proteases can be mimicked in vitro, e.g. by carrying out tryptic digests, in order to ascertain the sequences of preferred naturally occurring peptides (or substantially homologous peptides). Such studies indicate that preferred peptides or substantially homologous peptides of the present invention have Y or PY as the final (or C-terminal) amino acids. Other preferred peptides or substantially homologous peptides of the present invention have L or SL as the final (or C-terminal) amino acids. The Y residue can, for example, be found at position 9 (or a position corresponding to position 9) of the 9-mer core described herein. The L residue can, for example, be found at position 13 (or a position corresponding to position 13) in relation to the 9-mer core described herein. Thus, peptides with these residues at these positions are preferred, and other positions can have variant amino acid sequences, for example, the other positions can comprise any amino acid. More preferred peptides also contain the 9-mer core.

Other preferred peptides or substantially homologous peptides of the invention have Q or E residues at positions corresponding to positions 4, 5, 6 and 7 of the 9-mer core, and preferably have a Y residue at position 9 (or the corresponding residue). As described elsewhere herein, in such peptides an E residue at position 4 or 6, preferably 6 is preferred, in which case exemplary positions 4 to 7 (or corresponding residues) are QQEQ or EQEQ.

Other preferred peptides or substantially homologous peptides of the invention comprise a Q or preferably an E residue at the position corresponding to position−2 of the first epitope or T cell epitope, or at the position corresponding to position 3 of the second epitope or B cell epitope. In preferred embodiments this Q or E residue is the same residue in both the first (T cell) and second (B cell) epitopes.

Other preferred peptides or substantially homologous peptides of the invention comprise a Y residue at the position corresponding to position 2 of the first epitope or T cell epitope, or at the position corresponding to position 6 of the second epitope or B cell epitope. In preferred embodiments this Y residue is the same residue in both the first (T cell) and second (B cell) epitopes.

Other preferred peptides or substantially homologous peptides of the invention can have an aspartic acid (D) residue instead of a glutamic acid (E) residue at one or more positions.

For the avoidance of doubt, reference herein to deamidated versions or forms of the peptides etc., of the invention includes such forms of the peptides when generated by any process, i.e. it does not only refer to an E residue generated at a particular position by deamidation of a Q residue (although the peptides can be generated that way if appropriate), but refers to a peptide or amino acid sequence where an E residue is present in place of a Q residue at one or more positions by any appropriate means, e.g. by synthetic or recombinant creation of a peptide with those E residues. Thus, the reference to a deamidated version or form of a peptide as used herein at its broadest refers to a sequence with one or more E residues present in place of or instead of one or more of the Q residues present in the original peptide sequence.

Homology (e.g. sequence identity) may be assessed by any convenient method. However, for determining the degree of homology (e.g. identity) between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention having at least 60%, 70%, 80%, 85%, 88%, 90%, 92%, 94%, 95%, 96%, 97%, 98% or 99% homology, sequence identity etc. may be determined using the ALIGN program with default parameters (for instance available on Internet at the GENESTREAM network server, IGH, Montpellier, France).

In some embodiments, the present invention provides a peptide, e.g. an isolated peptide, that comprises (or consists of) an elongated or truncated version of a peptide, e.g. an isolated peptide, sequence disclosed herein (or a sequence substantially homologous thereto).

A peptide of the invention may comprise (or consist of) an elongated version of a peptide sequence disclosed herein, or an elongated version of an amino acid sequence substantially homologous to a peptide sequence disclosed herein. For example, one or more additional amino acids (e.g. at least 2, at least 3, at least 4, at least 5, at least 6, at least 7, at least 8, at least 9, at least 10, at least 15, at least 20, or at least 25 amino acids, or 1-5 or 1-10 or 1-20 amino acids) may be present at one end or both ends of the peptide sequence (or sequence substantially homologous thereto).

Nucleic acid molecules comprising (or consisting of) nucleotide sequences that encode the peptides or epitopes or conjugates or complexes of the present invention as defined herein, or nucleic acid molecules substantially homologous thereto, form yet further aspects of the invention. Vectors, or expression vectors, comprising the nucleic acid molecules of the invention, or cells, e.g. host cells, comprising said expression vectors or nucleic acid molecules form yet further aspects. Said vectors or cells can be used as alternatives to nucleic acid molecules in the various aspects of the invention described herein, e.g. in the therapeutic or diagnostic uses and methods.

The term “substantially homologous” as used herein in connection with a nucleic acid sequence includes sequences having at least 65%, 70% or 75%, preferably at least 80%, and even more preferably at least 85%, 90%, 95%, 96%, 97%, 98% or 99%, sequence identity to the starting nucleic acid sequence.

The term “nucleic acid sequence” or “nucleic acid molecule” as used herein refers to a sequence of nucleoside or nucleotide monomers composed of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid molecules may be double stranded or single stranded. The nucleic acid molecules may be wholly or partially synthetic or recombinant.

One or more nucleic acid molecules may be used to encode the desired sequences described here or elsewhere herein.

In embodiments of the invention, peptides or proteins consisting of or consisting essentially of the amino acid sequences of the peptides or epitopes or conjugates or complexes as described herein, or sequences substantially homologous thereto, or nucleic acid molecules encoding said peptides, or epitopes, or conjugates or complexes, or nucleic acid molecules substantially homologous thereto, form yet further aspects of the invention.

A further aspect of the invention provides a conjugate (or complex) comprising a peptide or epitope of the invention. Typically, the conjugate (or complex) is configured to present the peptide or epitope to T cells, e.g. CD4+ T cells. Typically, the conjugate (or complex) is configured to present the peptide or epitope to T cell receptors. The conjugate (or complex) may comprise at least one peptide or epitope of the invention as defined elsewhere herein coupled to (e.g. linked to or connected to, or joined to, or conjugated to, or bonded to), or otherwise associated with a second component or another entity.

Exemplary conjugates (or complexes) may comprise a peptide or epitope of the invention coupled to, e.g. physically coupled to, an MHC molecule, typically an MHC class II molecule (HLA molecule), to form a peptide-MHC (p-MHC) conjugate/complex. Such coupling can be achieved by methods known and described in the art. For example, the peptide or epitope of the invention can be coupled to an α or β chain (or a fragment or variant, e.g. a functional fragment or variant, for example, a truncated fragment or variant, thereof) of an MHC or MHC class II molecule, for example, via a peptide or other linker or means of linkage (e.g. a chemical linker or linkage). Thus, such linker or linkages are typically artificial, synthetic, or non-naturally occurring linkers or linkages. Peptide linkers with appropriate sequences, e.g. non-native or artificial sequences, can preferably and conveniently be provided as genetic fusions. Alternatively, click chemistry applications, e.g. sortase A, can be used to provide such linkages.

Such coupling or means of coupling can typically and conveniently be carried out via a permanent, covalent or irreversible linkage. Equally said coupling can be via an association, e.g. a physical association, e.g. where a peptide or epitope of the invention is bound to or associated with another entity or second component, e.g. an MHC molecule, without being joined by a physical linkage. Thus, free or isolated peptides of the invention can conveniently be loaded onto or bound to the MHC molecule.

Exemplary functional fragments or variants of MHC or MHC class II molecules are those that retain the ability to bind or otherwise associate with the peptides or epitopes of the invention. For example, a number of engineered HLA variants, e.g. truncated variants, are described in the art and any of these may be used.

As elsewhere herein, preferred MHC class II molecules for use in such conjugates (or complexes) are HLA-DQ2 molecules (or chains or fragments thereof), in particular HLA-DQ2.5 molecules or HLA-DQ2.2 molecules (or chains or fragments thereof). Such molecules thus comprise an MHC or HLA molecule, e.g. an HLA-DQ2.5 molecule, that is presenting (or “loaded” with) a peptide or an epitope, e.g. a gliadin epitope, e.g. an ω-gliadin epitope, of the invention. Put another way, such conjugates can be an HLA-DQ2.5-peptide or HLA-DQ2.2-peptide conjugate/complex (pMHC) in which a peptide or epitope of the invention is presented in the antigen binding groove (or accommodated in the antigen binding groove). Such pMHC complexes containing class II MHC molecules, e.g. HLA-DQ2.5-peptide or HLA-DQ2.2-peptide complexes can also be referred to as pMHCII. Such pMHC complexes can be produced in a soluble form, e.g. as isolated or recombinant molecules (e.g. recombinant soluble pMHC or pHLA molecules), or can be associated with or loaded on or into another entity or carrier or formulation, e.g. can be coated on or attached to solid supports, e.g. nanoparticles or other solid supports as described elsewhere herein, or can be expressed on cells, e.g. by recombinant expression, or can be associated with other particles, carriers or formulations such as lipid-based particles, carriers or formulations, e.g. liposomes or micelles. Such pMHC containing entities (including the multimers described below and elsewhere herein) can be used therapeutically, e.g. to induce T cell anergy, or can be used in diagnostic applications or methods of detection as described elsewhere herein, for example, in the detection (e.g. staining) of T cells, or, for example, to activate T cells.

As mentioned above, conjugates or complexes of the invention can contain more than one peptide or epitope of the invention coupled to a second entity, e.g. an MHC molecule. Thus, conjugates or complexes which comprise peptide-MHC (pMHC) multimers, e.g. with 2, 3, 4 or more peptide-MHC complexes (pMHC) are provided. Particularly preferred multimeric conjugates take the form of dimers or tetramers, e.g. molecules in which 2 or 4, respectively, peptide-MHC conjugates/complexes are provided together in a single complex or molecule. Any appropriate structure or design can be used for these conjugates with more than one peptide-MHC complex and appropriate formats are described in the art (for example dimers are described in Clemente-Casares et al., 2002, Nature Immunology 3, 383-391). In addition, tetramers can take any of the formats described in the art, for example, a format in which each pMHC complex is joined to a biotin containing moiety and a biotin-streptavidin interaction is used to join the four separate pMHC complexes together and form a tetramer (see, for example, Quarsten et al., J. Immunol., 2001, 167(9), 4861-8). In such multimeric conjugates it is preferred that each pMHC arm of the construct, e.g. each individual pMHC complex, retains the ability to bind to T cell receptors, e.g. on the surface of CD4+ T cells.

Other conjugates may comprise a peptide or epitope of the invention and a peptide carrier, wherein said peptide or epitope is coupled to said peptide carrier. Peptide carriers typically enhance immunogenicity or can be chosen to enhance immunogenicity. Thus, such conjugates are particularly useful if it is desired to induce an antigenic response to the peptide or epitope. Such conjugates are therefore appropriate to generate or raise antibodies to the peptide or epitope of the invention. As described elsewhere herein, such antibodies that can bind or specifically bind to the peptide or epitope of the invention provide a further embodiment of the invention.

One or more nucleic acid molecules encoding such conjugates or complexes are also provided.

As set out above, in some embodiments, peptides (or conjugates or complexes, e.g. pMHC) of the invention may be present on (i.e. attached to or bound to) a solid support (e.g. a particle or planar support, for example, a bead or microbead or nanoparticle or plate or microtitre plate). Thus, in one aspect the present invention provides a solid support, having attached thereto (either directly or indirectly attached thereto) a peptide or conjugate or complex of the invention. Typically, multiple copies will be attached. Such solid supports are in particular suitable for use or used in in vitro diagnostic applications.

As set out above and described elsewhere herein, in some embodiments, peptides (or conjugates or complexes, e.g. pMHC) of the invention may be present in association with other entities, formulations or carriers, for example, lipid-based formulations or carriers such as liposomes or micelles. Thus, carriers or formulations, e.g. lipid-based carriers or formulations, comprising peptides, conjugates or complexes of the invention form a yet further aspect.

A yet further aspect provides an antigen presenting cell (APC) or other cell comprising, presenting or loaded with a peptide or epitope or conjugate or complex of the invention. In such embodiments the peptide or epitope of the invention is typically located on or associated with the cell surface. Any appropriate method of preparing such cells can be used. Thus, if the cell naturally expresses or comprises MHC class II molecules on its surface which are capable of binding to the peptide or epitope, the cells can be externally loaded with the peptide or epitope of the invention. Alternatively, the cells can be engineered to express the peptide or epitope of the invention, e.g. by recombinant means. For example, the cells can be transfected with a construct which results in the expression of an appropriate pMHC (e.g. a pMHC conjugate or complex of the invention) on the surface, e.g. such cells can comprise a nucleic acid molecule of the invention. Such cells could be used therapeutically, e.g. to induce T cell anergy, as described elsewhere herein.

Any appropriate cell type can be used. Preferred APCs might take the form of dendritic cells, macrophages or B-lymphocytes, in particular plasma cells.

A yet further aspect of the invention provides a binding protein that binds or specifically binds to a peptide or epitope or complex or conjugate of the invention. In particular, binding proteins that specifically bind to a peptide or epitope or complex or conjugate of the invention are preferred. Binding proteins that bind or specifically bind to a deamidated (or E residue containing) peptide or epitope or complex or conjugate of the invention are also provided and are preferred in some aspects.

In some embodiments, said binding proteins are present or expressed on a cell (cell surface), e.g. can be a cell, e.g. a eukaryotic cell such as a T cell or NK cell, comprising or expressing said binding protein, e.g. can be CAR T cells or TCR T cells or TCR-NK cells or CAR-NK cells.

Thus, a yet further aspect of the present invention, provides a method of expressing a binding protein of the invention on the surface of a cell, e.g. a eukaryotic cell such as a T cell or NK cell. Said methods may conveniently comprise the step of providing the cell with a nucleic acid molecule or construct encoding said binding protein and allowing expression of said binding protein to occur on the surface of the cell.

As the binding proteins of the invention can target peptides and epitopes associated with CeD, in some embodiments the binding proteins of the invention may be associated with or conjugated to (attached to) other entities, e.g. other useful therapeutic entities or moieties.

For example, the binding proteins (e.g. antibodies or T cell receptors) of the invention may be conjugated to or associated with a toxic or cytotoxic moiety, e.g. in the form of an antibody drug conjugate (ADC), or some other payload such as an inhibitory RNA molecule, e.g. an siRNA molecule. In some embodiments, the binding proteins of the present invention, e.g. antibody molecules, can be internalised into target cells or enable internalisation into target cells. As the binding proteins of the present invention can target pMHC complexes on APCs, if the binding proteins are then internalised, this provides a convenient way for ensuring that the payload enters the target cells. Thus, depending on the payload chosen, the APCs expressing the pMHC target (e.g. APCs associated with CeD) can be killed or deleted (e.g. if the payload is a cytotoxic molecule or an appropriate inhibitory RNA/siRNA molecule) or gene or protein levels can be altered (e.g. if the payload is an inhibitory RNA/siRNA). Such conjugates therefore have therapeutic uses as described herein, e.g. in the treatment of CeD.

Alternatively, the binding proteins (e.g. antibodies, e.g. TCR-like antibodies, or T cell receptors) of the invention may be conjugated to or associated with a second binding protein with specificity for another entity, e.g. effector cells, which can then be recruited. Appropriate effector cells to which the second binding protein has specificity could be any type of cell, for example, T cells or NK cells. Thus, bispecific molecules comprising the binding proteins of the invention, for example, bispecific antibodies and Cell Engagers such as bispecific T cell engager molecules (BiTEs) are also provided. Appropriate second binding proteins, e.g. antibodies or T cell receptors, can be readily selected depending on the entity or effector cell being targeted, and examples are well known and described in the art. For example, where the target is a T cell or an NK cell, then the second binding protein, e.g. antibody or T cell receptor, is selected to bind to a T cell surface protein (e.g. CD3 or CD16, e.g. in the form of an anti-CD3 or anti-CD16 unit such as an anti-CD3 or anti-CD16 antibody) or an NK cell surface protein (e.g. NKG2D, e.g. in the form of an anti-NKG2D unit such as an anti-NKG2D antibody) as appropriate.

Binding proteins of the invention could also be associated with or conjugated to (attached to) entities which can be detected or enable detection, for example, can be associated with labels or other detectable moieties. Such labelled or detectable binding proteins (e.g. antibodies) could then be used to detect peptides, epitopes or pMHC complexes of the invention, e.g. could be used to detect APCs displaying peptides or epitopes of the invention in association with MHC molecules. In addition, as the binding proteins of the invention can target peptides, epitopes or pMHC complexes associated with CeD, e.g. on the surface of antigen presenting cells, the binding proteins, e.g. antibodies, can be used to block or inhibit the interaction of T cells (e.g. gluten specific T cells or pathogenic T cells) with the pMHC complex and in turn inhibit or reduce T cell activation or proliferation.

The binding proteins of the invention thus also have therapeutic uses and can be used in therapy, e.g. as described elsewhere herein.

A preferred such binding protein is a T cell receptor (TCR). Said T cell receptor can be present either on a cell, e.g. can be a cell, e.g. a eukaryotic cell, comprising or expressing said T cell receptor on its surface, e.g. can be a T cell, or NK cell or other cell type expressing a TCR, e.g. by recombinant means, or can be a soluble T cell receptor (TCR), e.g. a TCR that is not associated with a cell membrane, for example, produced by recombinant means. Thus, further embodiments of the invention provide cells, e.g. T cells or NK cells, which have or express cell surface TCRs which can bind or specifically bind to a peptide or epitope or complex or conjugate of the invention. Other embodiments provide a soluble TCR which can bind or specifically bind to a peptide or epitope or complex or conjugate of the invention. In particular, TCRs or T cells which can bind or specifically bind to pMHC complexes or molecules of the invention are preferred. TCRs or T cells which show specific binding are also preferred. Fragments of TCRs are also included providing they retain functional activity such as the ability to bind or specifically bind to pMHC complexes or molecules of the invention. TCRs that bind or specifically bind to a deamidated (or E residue containing) peptide or epitope or complex or conjugate of the invention are also provided and are preferred in some aspects.

Another preferred binding protein is or comprises an antibody or antigen binding domain of an antibody that specifically binds to a peptide or epitope or complex or conjugate of the invention. Antibodies or antigen binding domains of antibodies that bind or specifically bind to a pMHC or pHLA complex are sometimes referred to as TCR-like antibodies. A binding protein that is or comprises an antibody or antigen binding domain of an antibody that binds (or specifically binds) to a peptide or epitope of the invention, in particular a peptide comprising a T cell epitope of the invention, for example, comprising a T cell epitope of the invention having the 9-mer core sequence but not comprising a B cell epitope of the invention (e.g. in a naked, isolated or uncomplexed form, as opposed to in a complex or conjugate, e.g. pMHC) is also provided. A binding protein that is or comprises an antibody or antigen binding domain of an antibody that binds (or specifically binds) to a complex or conjugate of the invention (e.g. a peptide of the invention in the form of pMHC, as opposed to a peptide of the invention in a naked, isolated or uncomplexed form) is also provided. In such embodiments, said peptide in the pMHC preferably comprises a T cell epitope of the invention, for example, comprises a T cell epitope of the invention having the 9-mer core sequence but does not comprise a B cell epitope of the invention.

Such binding proteins can be present on a cell, e.g. can be a cell, e.g. a eukaryotic cell, comprising or expressing said antibody or antigen binding domain of an antibody on its surface, e.g. can be a T cell, e.g. in the form of a CAR T cell, or can be a soluble or isolated binding protein, e.g. an antibody or antigen binding domain of an antibody that is not associated with a cell membrane, for example, produced by recombinant means.

Preferably, the binding protein comprising an antigen binding domain of an antibody is an antibody or an antigen binding fragment thereof.

As will be understood by those in the art, the immunological binding reagents encompassed by the term “antibody” includes or extends to all antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region comprising one or more CDRs and framework (FR) regions (or one or more VH and/or VL regions), and this term includes antibody fragments that comprise an antigen binding domain such as Fab′, Fab, F(ab′)₂, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lambda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); and small antibody mimetics.

As used herein, the term “specifically binds” or “specifically recognises” in the context of a peptide or epitope or complex or conjugate of the invention means those binding proteins (e.g. TCRs, antibodies or antigen binding domains of antibodies) that are capable of binding to a peptide or epitope of the invention, e.g. a peptide or epitope comprising the sequence PYPQQQQPY (SEQ ID NO:8) or a deamidated (or E residue containing) version thereof, or to a complex or conjugate of the invention, e.g. a complex or conjugate comprising said peptide or epitope of the invention loaded or presented on HLA-DQ2.5 or HLA-DQ2.2, and which do not cross-react (or do not bind) or do not significantly cross-react (or do not significantly bind) with other peptides, for example, other naked, isolated or unconjugated peptides or other peptides loaded or presented on HLA-DQ2.5 or HLA-DQ2.2 (e.g. other celiac disease associated peptides, or other gliadin or gliadin-derived peptides, or variants of gliadin derived peptides, or other gluten-derived peptides). Preferably, binding proteins (e.g. TCRs, antibodies or antigen binding domains of antibodies) do not cross-react (or do not bind) or do not significantly cross-react (or do not significantly bind) with DQ2.5:DQ2.5-glia-α1a. Other preferred binding proteins (e.g. TCRs, antibodies or antigen binding domains of antibodies) do not cross-react (or do not bind) or do not significantly cross-react (or do not significantly bind) to peptides comprising B cell epitopes as described herein but not comprising the core 9-mer T cell epitopes of the invention, e.g. naked, isolated or unconjugated peptides comprising B cell epitopes as described herein, or peptides comprising B cell epitopes as described herein loaded or presented on MHC molecules (pMHC), e.g. HLA-DQ2.5 or HLA-DQ2.2, but not comprising the core 9-mer T cell epitopes of the invention.

HLA-DQ2.5:DQ2.5-glia-α1a means an HLA-DQ2.5 molecule that is presenting (or 5 “loaded” with) a DQ2.5-glia-α1a epitope (PFPQPELPY (SEQ ID NO:4)). Put anotherway, HLA-DQ2.5:DQ2.5-glia-α1a means an HLA-DQ2.5-peptide complex (pMHC) in which the DQ2.5-glia-α1a epitope is presented in the antigen binding groove (or accommodated in the antigen binding groove).

Other preferred binding proteins (e.g. TCRs, antibodies or antigen binding domains of antibodies) are specific to deamidated (or E residue containing) peptides of the invention. Such binding proteins bind to deamidated (or E residue containing) peptides of the invention either alone (in naked or isolated form) or when associated with MHC molecules but do not cross-react (or do not bind) or do not significantly cross-react (or do not significantly bind) to non-deamidated or healthy forms of the peptides of the invention.

In some embodiments, the binding proteins of the invention, in particular antibodies or antigen binding domains of antibodies, bind to their antigen (e.g. a pMHC as described herein comprising a peptide or epitope of the invention) with a binding affinity (K_{D}) of 1 nM or less (e.g. 1 pM to 1 nM, or 10 pM to 1 nM, or 20 pM to 1 nM, or 50 pM to 1 nM, or 100 pM to 1 nM, or 1 pM to 500 pM, or 10 pM to 500 pM, or 20 pM to 500 pM, or 50 pM to 500 pM, or 100 pM to 500 pM, or 1 pM to 100 pM, or 10 pM to 100 pM, or 20 pM to 100 pM, or 50 pM to 100 pM), preferably 900 pM or less, 800 pM or less, 700 pM or less, 600 pM or less, 500 pM or less, 400 pM or less, 300 pM or less, 200 pM or less (e.g. 200, 190, 180, 170, 160, 150, 140, 130, 120, 110, 100, 90, 80, 70, 60, 50, 45, 40, 35, 30, 25, 20, 15 or 10 pM, or less), or 100 pM or less (e.g. 90, 80, 70, 60, 50, 45, 40, 35, 30, 25, 20, 15 or 1 pM, or less). Preferably, the above-mentioned binding affinities and affinity range values apply when the antigen binding protein is in the scFv format or in the Fab format or in a whole antibody format.

Binding affinities (K_{D}) can be measured by any appropriate technique which would be well known to a person skilled in the art. A convenient technique would be to use surface plasmon resonance (SPR), e.g. BIAcore,

The binding proteins of the invention thus recognise or bind to residues found in the peptides or epitopes of the invention. Thus, although the binding proteins may also contact or interact with some residues outside the peptide or epitope, the binding proteins of the invention do not bind to MHC molecules alone, e.g. MHC molecules that are empty or unloaded with peptide.

In some embodiments, the binding protein is not or does not comprise an antibody or an antigen binding fragment thereof. In other embodiments, the binding protein is not or does not comprise an antigen binding domain of an antibody.

In some embodiments the binding protein is or comprises an antibody or an antigen binding domain of an antibody, with the proviso that said binding protein does not bind to HLA-DQ2.5:DQ2.5 presenting the α1a gliadin peptide. In other embodiments, said binding protein also does not bind to HLA-DQ2.2 presenting the α1a gliadin peptide.

In some embodiments the binding protein is or comprises an antibody or an antigen binding protein (or an antigen binding domain of an antibody), with the proviso that said binding protein is not an antibody or an antigen binding protein or domain which binds to HLA-DQ2.5:DQ2.5 presenting the α1a gliadin peptide, said antibody or antigen binding protein (or an antigen binding domain of an antibody) comprising at least one light chain variable domain and at least one heavy chain variable domain, each domain comprising three complementarity determining regions (CDRs), wherein said antibody or antigen binding protein or domain comprises:

a variable heavy (VH) CDR1 comprising the amino acid sequence of GDSVSSNSAA (SEQ ID NO:40), or a sequence containing 1, 2 or 3 amino acid substitutions, additions or deletions relative to said sequence;

a variable heavy (VH) CDR2 comprising the amino acid sequence of TYYRSKWYN (SEQ ID NO:41), or a sequence containing 1, 2 or 3 amino acid substitutions, additions or deletions relative to said sequence;

a variable heavy (VH) CDR3 comprising the amino acid sequence of ARDX₄X₅X₆GWX₉X₁₀YGMDV (SEQ ID NO:42), wherein
X₄ can be any amino acid, preferably S or R (SEQ ID NO:43);
X₅ can be any amino acid, preferably S or T (SEQ ID NO:43);
X₆ can be any amino acid, preferably S or T (SEQ ID NO:43);
X₉ s can be any amino acid, preferably H or N or G (SEQ ID NO:43); and
X₁₀ can be any amino acid, preferably P or A (SEQ ID NO:43);

a variable light (VL) CDR1 comprising the amino acid sequence of HDISSY (SEQ ID NO:44), or a sequence containing 1, 2 or 3 amino acid substitutions, additions or deletions relative to said sequence;

a variable light (VL) CDR2 comprising the amino acid sequence of AAS (SEQ ID NO:45) or a sequence containing 1 amino acid substitution, addition or deletion relative to said sequence; and

a variable light (VL) CDR3 comprising the amino acid sequence of QX₂LNSYPLX₉X₁₀ (SEQ ID NO:46) wherein
X₂ can be any amino acid, preferably D or Q (SEQ ID NO:47);
X₉ can be any amino acid or no amino acid, preferably no amino acid or L (SEQ ID NO:47); and
X₁₀ can be any amino acid or no amino acid, preferably no amino acid or T (SEQ ID NO:47).

In other embodiments, antibodies or antigen binding proteins (or an antigen binding domain of an antibody) which bind to HLA-DQ2.5:DQ2.5 presenting the α1a gliadin peptide, and which comprise one or more (e.g. 1, 2, 3, 4, 5 or all 6) of the above CDRs, are also excluded from the scope of the present invention.

In general, in some embodiments, antibody molecules with CDR regions as defined above are excluded from the scope of the present invention.

In some embodiments the binding protein is or comprises an antibody or an antigen binding protein (or an antigen binding domain of an antibody), with the proviso that said binding protein is not an antibody or an antigen binding protein (or an antigen binding domain of an antibody) which binds to HLA-DQ2.5:DQ2.5 presenting the α1a gliadin peptide, said antibody or antigen binding protein (or an antigen binding domain of an antibody) comprising at least one light chain variable domain and at least one heavy chain variable domain, each domain comprising three complementarity determining regions (CDRs), wherein said antibody or antigen binding protein (or an antigen binding domain of an antibody) comprises:

a variable heavy (VH) CDR1 comprising the amino acid sequence of GDSVSSNSAA (SEQ ID NO:40), or a sequence containing 1, 2 or 3 amino acid substitutions, additions or deletions relative to said sequence;

a variable heavy (VH) CDR2 comprising the amino acid sequence of TYYRSKWYN (SEQ ID NO:41), or a sequence containing 1, 2 or 3 amino acid substitutions, additions or deletions relative to said sequence;

a variable heavy (VH) CDR3 comprising the amino acid sequence of ARDX₄X₅X₆GWX₉X₁₀YGMDV (SEQ ID NO:42), wherein
X₄ can be any amino acid, preferably S or R (SEQ ID NO:43);
X₅ can be any amino acid, preferably S or T (SEQ ID NO:43);
X₆ can be any amino acid, preferably S or T (SEQ ID NO:43);
X₉ can be any amino acid, preferably H or N or G (SEQ ID NO:43); and
X₁₀ can be any amino acid; preferably P or A (SEQ ID NO:43);
more preferably wherein said (VH) CDR 3 comprises the amino acid sequence of ARDSSSGWHPYGMDV (SEQ ID NO:48);

a variable light (VL) CDR1 comprising the amino acid sequence of HDISSY (SEQ ID NO:44) or a sequence containing 1, 2 or 3 amino acid substitutions, additions or deletions relative to said sequence;

a variable light (VL) CDR2 comprising the amino acid sequence of AAS (SEQ ID NO:45) or a sequence containing 1 amino acid substitution, addition or deletion relative to said sequence; and

a variable light (VL) CDR3 comprising the amino acid sequence of QDLNSYPL (SEQ ID NO:49) or a sequence containing 1, 2 or 3 amino acid substitutions, additions or deletions relative to said sequence.

In other embodiments, antibodies or antigen binding proteins (or an antigen binding domain of an antibody) which bind to HLA-DQ2.5:DQ2.5 presenting the α1a gliadin peptide, and which comprise one or more (e.g. 1, 2, 3, 4, 5 or all 6) of the above CDRs, are also excluded from the scope of the present invention.

In general, in some embodiments, antibody molecules with CDR regions as defined above are excluded from the scope of the present invention.

In some embodiments the binding protein is or comprises an antibody or an antigen binding protein (or an antigen binding domain of an antibody), with the proviso that said binding protein is not an antibody or an antigen binding protein (or an antigen binding domain of an antibody) which binds to HLA-DQ2.5:DQ2.5 presenting the α1a gliadin peptide, said antibody or antigen binding protein (or an antigen binding domain of an antibody) comprising at least one light chain variable domain and at least one heavy chain variable domain, each domain comprising three complementarity determining regions (CDRs), wherein said antibody or antigen binding protein (or an antigen binding domain of an antibody) comprises:

a variable heavy (VH) CDR1 that comprises the amino acid sequence of GDSVSSNSAA (SEQ ID NO:40) or a sequence containing 1, 2 or 3 amino acid substitutions, additions or deletions relative to said sequence;

a VH CDR2 that comprises the amino acid sequence of TYYRSKWYN (SEQ ID NO:41) or a sequence containing 1, 2 or 3 amino acid substitutions, additions or deletions relative to said sequence;

a VH CDR3 that comprises the amino acid sequence of ARDRTTGWHPYGMDV (SEQ ID NO:50) or a sequence containing 1, 2 or 3 amino acid substitutions, additions or deletions relative to said sequence;

a variable light (VL) CDR1 that comprises the amino acid sequence of HDISSY (SEQ ID NO:44) or a sequence containing 1, 2 or 3 amino acid substitutions, additions or deletions relative to said sequence;

a VL CDR2 that comprises the amino acid sequence of AAS (SEQ ID NO:45) or a sequence containing 1 amino acid substitution, addition or deletion relative to said sequence, and

a VL CDR3 that comprises the amino acid sequence of QDLNSYPL (SEQ ID NO:49) or a sequence containing 1, 2 or 3 amino acid substitutions, additions or deletions relative said sequence.

In other embodiments, antibodies or antigen binding proteins (or an antigen binding domain of an antibody) which bind to HLA-DQ2.5:DQ2.5 presenting the α1a gliadin peptide, and which comprise one or more (e.g. 1, 2, 3, 4, 5 or all 6) of the above CDRs, are also excluded from the scope of the present invention.

In general, in some embodiments, antibody molecules with CDR regions as defined above are excluded from the scope of the present invention.

Antibodies with all 6 of the CDR sequences:

| | | |
|---|---|---|
| SEQ ID NO: 40 | Heavy CDR1 | GDSVSSNSAA |
| SEQ ID NO: 41 | Heavy CDR2 | TYYRSKWYN |
| SEQ ID NO: 48 | Heavy CDR3 | ARDSSSGWHPYGMDV |
| SEQ ID NO: 44 | Light CDR1 | HDISSY |
| SEQ ID NO: 45 | Light CDR2 | AAS |
| SEQ ID NO: 49 | Light CDR3 | QDLNSYPL |

are also referred to herein as the 107 antibody. Such antibodies (or other binding proteins with all 6 of these CDR sequences), for example, antibodies as defined in Table 1, for example, antibodies with the VH and VL domains as outlined in Table 1, or one or more of the other sequences as outlined in Table 1, are excluded from the scope of the present invention.

In other embodiments, antibodies (or other binding proteins) with VH and/or VL domains, that have at least 60%, 70%, 80%, 85%, 88%, 90%, 92%, 94%, 95%, 96%, 97%, 98% or 99% sequence identity to the given amino acid sequence in Table 1 are excluded, or antibodies (or other binding proteins) with a set of 6 CDR domains that have at least 60%, 70%, 80%, 85%, 88%, 90%, 92%, 94%, 95%, 96%, 97%, 98% or 99% sequence identity to the set (combined set) of 6 CDRs of Table 1, i.e. the set of CDRs taken as a whole, are excluded.

In other embodiments, antibodies (or other binding proteins) with VH and/or VL domains, that have less than 60%, 70%, 80%, 85%, 88%, 90%, 92%, 94%, 95%, 96%, 97%, 98% or 99% sequence identity to the given amino acid sequence in Table 1 are provided, or antibodies (or other binding proteins) with a set of 6 CDR domains that have less than 60%, 70%, 80%, 85%, 88%, 90%, 92%, 94%, 95%, 96%, 97%, 98% or 99% sequence identity to the set (combined set) of 6 CDRs of Table 1, i.e. the set of CDRs taken as a whole, are provided.

Antibodies with all 6 of the CDR sequences:

| | | |
|---|---|---|
| SEQ ID NO: 40 | Heavy CDR1 | GDSVSSNSAA |
| SEQ ID NO: 41 | Heavy CDR2 | TYYRSKWYN |
| SEQ ID NO: 50 | Heavy CDR3 | ARDRTTGWHPYGMDV |
| SEQ ID NO: 44 | Light CDR1 | HDISSY |
| SEQ ID NO: 45 | Light CDR2 | AAS |
| SEQ ID NO: 49 | Light CDR3 | QDLNSYPL |

are also referred to herein as the 4.7C antibody. Such antibodies (or other binding proteins with all 6 of these CDR sequences), for example, antibodies as defined in Table 2, for example, antibodies with the VH and VL domains as outlined in Table 2, or one or more of the other sequences as outlined in Table 2, are excluded from the scope of the present invention.

In other embodiments, antibodies (or other binding proteins) with VH and/or VL domains that have at least 60%, 70%, 80%, 85%, 88%, 90%, 92%, 94%, 95%, 96%, 97%, 98% or 99% sequence identity to the given amino acid sequence in Table 2 are excluded, or antibodies (or other binding proteins) with a set of 6 CDR domains that have at least 60%, 70%, 80%, 85%, 88%, 90%, 92%, 94%, 95%, 96%, 97%, 98% or 99% sequence identity to the set (combined set) of 6 CDRs of Table 2, i.e. the set of CDRs taken as a whole, are excluded.

In other embodiments, antibodies (or other binding proteins) with VH and/or VL domains that have less than 60%, 70%, 80%, 85%, 88%, 90%, 92%, 94%, 95%, 96%, 97%, 98% or 99% sequence identity to the given amino acid sequence in Table 2 are provided, or antibodies (or other binding proteins) with a set of 6 CDR domains that have less than 60%, 70%, 80%, 85%, 88%, 90%, 92%, 94%, 95%, 96%, 97%, 98% or 99% sequence identity to the set (combined set) of 6 CDRs of Table 2, i.e. the set of CDRs taken as a whole, are provided.

In other embodiments, antibodies (or other binding proteins), as described in WO2019/158602, are excluded.

In other embodiments, antibodies (or other binding proteins) with a VH of:

(SEQ ID NO: 51)
EVQLVESGGGLVKPGGSLRLSCAASGFTFSNAWFNWVRQAPGKGLEWVGR
IKTNTDGGTTDYAAPVKGRFTISRDDSKNTLYLQMNSLKTEDTAVYYCTT
GEPLVNHITILDYWGQGTLVTVSS,

and/or a VL of:

(SEQ ID NO: 52)
DIVMTQSPDSLAVSLGERATINCKSSQSVLYSSNNKNYLAWYQQKPGQPP
KLLIYWASTRESGVPDRFSGSGSGTDFTLTISSLQAEDVAVYYCQQYYRT
PPLTFGGGTKVEIK,

or the antibody referred to herein as 1E03, are excluded from the scope of the present invention.

In other embodiments, antibodies (or other binding proteins) with VH and/or VL domains that have at least 60%, 70%, 80%, 85%, 88%, 90%, 92%, 94%, 95%, 96%, 97%, 98% or 99% sequence identity to the given amino acid sequence are excluded.

In other embodiments, antibodies (or other binding proteins) with VH and/or VL domains that have less than 60%, 70%, 80%, 85%, 88%, 90%, 92%, 94%, 95%, 96%, 97%, 98% or 99% sequence identity to the given amino acid sequence are provided.

Preferred and exemplary peptides or epitopes or complexes or conjugates of the invention are described elsewhere herein and apply mutatis mutandis to these aspects relating to binding proteins of the invention. For example, in some embodiments binding proteins, e.g. antibodies or binding proteins comprising antigen binding domains of antibodies, or TCRs, which bind or specifically bind to deamidated (or E residue containing) peptides or peptide complexes (e.g. pMHC) of the invention are preferred.

One or more nucleic acid molecules encoding the binding proteins of the invention are also provided.

A yet further aspect of the invention provides a method of producing (or manufacturing or isolating or identifying or generating) a binding protein, e.g. an antibody, a binding protein comprising an antigen binding domain of an antibody, a TCR (or T cell), of the invention, said method employing a peptide or epitope or conjugate or complex (e.g. pMHC complex) of the invention. Alternatively viewed, the present invention provides the use of a peptide or epitope or conjugate or complex (e.g. pMHC complex) of the invention for the identification (or isolation or generation or production) of a binding protein, e.g. an antibody, a binding protein comprising an antigen binding domain of an antibody, a TCR (or T cell), of the invention.

Thus, a further aspect of the invention provides a method of producing (or manufacturing or isolating or identifying or generating) an antibody of the present invention, or an antibody which can bind to a peptide or epitope or conjugate or complex of the present invention, comprising a step of immunizing a non-human animal (e.g. a rabbit) with a peptide (or epitope or complex or conjugate) of the invention. Preferred methods include a step of obtaining from said animal antibodies that have been generated (or raised) against the peptide (or epitope or complex or conjugate) of the invention, and optionally a step of purification of the antibody product and/or formulating the antibody or product (or antigen binding fragment thereof) into a composition including at least one additional component, such as a carrier or excipient, e.g. a pharmaceutically acceptable carrier or excipient.

A further aspect of the invention provides a method of producing (or manufacturing or isolating or identifying or generating) an antibody of the present invention, or an antibody which can bind to a peptide or epitope or conjugate or complex of the present invention, by employing a peptide, epitope, complex, or conjugate of the invention in hybridoma technology (e.g. conventional hybridoma technology). Alternatively viewed, the present invention provides the use of a peptide, epitope, complex, or conjugate of the invention for the identification (or isolation or generation or production) of an antibody of the invention, or an antibody which can bind to a peptide or epitope or conjugate or complex of the present invention, using hybridoma technology. In some embodiments, a non-human animal (e.g. mouse) is immunized with a peptide, epitope, complex, or conjugate of the invention, spleen cells are isolated from said immunized animal (e.g. mouse) and fused with myeloma cells (e.g. mouse myeloma cells) lacking HGPRT expression (such myeloma cells are unable to grow in HAT containing media) and hybrid (i.e. fused or hybridoma) cells are selected using hypoxanthine, aminopterin and thymine (HAT) containing media. Only fused cells grow in HAT containing media.

A further aspect of the invention provides a method of identifying (or isolating or generating) an antibody of the invention, or an antibody which can bind to a peptide or epitope or conjugate or complex of the present invention, which method employs a peptide or epitope or conjugate or complex (e.g. pMHC complex) of the invention to screen an antibody library, e.g. using phage display technology (with a phage display antibody library).

Alternatively viewed, the present invention provides the use of a peptide or epitope or conjugate or complex of the invention for the identification (or isolation or generation or production) of an antibody of the invention, or an antibody which can bind to a peptide or epitope or conjugate or complex of the present invention, using phage display technology (with a phage display antibody library). Appropriate phage display techniques and libraries are well known and standard in the art. For example, in some embodiments, a peptide, epitope, complex, or conjugate of the invention (typically immobilised on a solid support such as a bead or microbead or plate or microtitre plate) is contacted with a phage display library (e.g. a bacteriophage library, typically a filamentous bacteriophage library such as an M13 or fd phage library) which displays (or presents or expresses) on the phage surface a library of antibodies or antibody fragments such as scFv or Fab fragments. Any suitable phage display antibody library may be used and the skilled person is familiar with these (and, for example, there are commercially available phage display antibody libraries). The bound phage is then eluted and the identity of the displayed antibody may be readily determined by isolating and sequencing the phage's nucleic acid (or at least the portion of the nucleic acid that encodes the displayed antibody). In some embodiments, after elution of the bound phage, one or more (e.g. 1, 2, 3, 4, 5 or more) additional rounds of contacting and eluting is performed prior to identifying the displayed antibody of the bound phage. Such additional rounds typically further enrich the library.

Alternative screening methods to identify (or isolate or generate) an antibody or T cell/TCR of the invention, or an antibody which can bind to a peptide or epitope or conjugate or complex of the present invention, may involve using a peptide or epitope or conjugate or complex (e.g. pMHC complex) of the invention to screen other appropriate sources of antibodies or T cells/TCRs, e.g. appropriate antibody or T cell containing samples from subjects, in particular human subjects, e.g. CeD subjects, for antibodies or T cells/TCRs that can bind or specifically bind to a peptide or epitope or conjugate or complex (e.g. pMHC complex) of the invention. Appropriate and exemplary samples are described elsewhere herein. Alternatively, sequence-based screening methods using NGS and bioinformatics to identify such targets could be used (see e.g. Nannini et al., 2021, MAbs. 13(1):1864084).

Preferred conjugates or complexes for use in such methods are pMHC complexes or conjugates. Preferred antibodies or T cells/TCRs of the invention generated by such methods are described elsewhere herein, for example, are antibodies or T cells/TCRs that specifically bind to a peptide or epitope or conjugate or complex (e.g. pMHC complex) of the invention.

A yet further aspect provides a method of detecting the peptides or epitopes or conjugates or complexes (e.g. pMHC complexes) of the invention using a binding protein, e.g. an antibody, or protein comprising an antigen binding domain of an antibody, or a T cell/TCR, e.g. a binding protein of the invention, that recognises or specifically recognises said peptide or epitope or conjugate or complex (e.g. pMHC complex). Said method comprises, for example, contacting a sample potentially containing said peptide, epitope, conjugate or complex, with said binding protein under conditions effective to allow the formation of complexes between the binding protein and the peptide, epitope, conjugate or complex, and detecting the complexes so formed.

In another aspect, the present invention provides a composition comprising a peptide (or conjugate or complex) of the invention or a nucleic acid molecule (or molecules) encoding such peptides or conjugates or complexes or a cell or other vehicle (e.g. solid support, particles/nanoparticles, lipid-based or other formulations as described herein) presenting or loaded with a peptide, conjugate or complex of the invention. Such compositions may further comprise (e.g. be in admixture with) a suitable diluent, carrier, excipient and/or preservative (e.g. a pharmaceutically acceptable diluent, carrier, excipient and/or preservative). Thus, in some embodiments said compositions are pharmaceutically acceptable compositions.

In some embodiments, peptides or epitopes of the invention are used (e.g. used therapeutically or for detection or diagnosis) in their “naked” unconjugated or uncomplexed form, e.g. as isolated or “free” peptides or epitopes.

The compositions according to the invention may be presented, for example, in a form suitable for oral, nasal, parenteral, intravenal, topical or rectal administration. In a preferred embodiment, compositions according to the invention are presented in a form suitable for intravenal administration. In some embodiments, compositions according to the invention are presented in a form suitable for intraperitoneal (i.p.) administration. In some embodiments, compositions according to the invention are presented in a form suitable for injection.

The active compounds defined herein may be presented in the conventional pharmacological forms of administration, such as tablets, coated tablets, nasal sprays, solutions, emulsions, nanoformulations (nanoparticles), liposomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions may then be filled into injection vials or ampoules.

Nasal sprays may be formulated similarly in aqueous solution and packed into spray containers, either with an aerosol propellant or provided with means for manual compression.

The pharmaceutical compositions (formulations) of the present invention are preferably administered parenterally by any suitable means. Intravenous administration is preferred. In some embodiments, administration is intraperitoneal (i.p.) administration.

Parenteral administration may be performed by subcutaneous, intramuscular or intravenous injection by means of a syringe. Alternatively, parenteral administration can be performed by means of an infusion pump. A further option is a composition which may be a powder or a liquid for the administration of the peptide or peptide containing complex or conjugate in the form of a nasal or pulmonal spray. As a still further option, the peptide or peptide containing complex or conjugate of the invention can also be administered transdermally, e.g. from a patch, optionally an iontophoretic patch, or transmucosally, e.g. buccally.

Suitable dosage units can be determined by a person skilled in the art.

The compositions, e.g. pharmaceutical compositions, may additionally comprise further active ingredients (e.g. as described elsewhere herein) in the context of co-administration regimens or combined (combination therapy) regimens. For example, the therapeutic methods and uses of the invention may be used in combination with any other appropriate therapeutic regime or agent useful for the treatment or prevention of CeD.

Vaccines comprising one or more of the peptides, epitopes, complexes or conjugates of the invention, or nucleic acid molecules encoding such entities, or cells or other vehicles as described elsewhere herein of the invention, or compositions or formulations comprising such peptides, epitopes, complexes, conjugates, nucleic acid molecules or cells or other vehicles as described elsewhere herein form yet further aspects of the invention. Such vaccine or vaccine compositions, formulations or vehicles, optionally further comprise a pharmaceutically acceptable carrier and/or an adjuvant.

As the epitopes or peptides or complexes or conjugates of the invention, in particular the various deamidated (or E residue containing) epitopes or peptides or complexes or conjugates of the invention, can be associated with celiac disease, such epitopes or peptides or complexes or conjugates (or more specifically the detection of such epitopes or peptides or complexes or conjugates) can be used for diagnosis of CeD.

The present invention further provides a method for diagnosing CeD in a subject, said method comprising contacting a sample from the subject with a peptide or epitope or complex or conjugate of the invention and determining whether said peptide or epitope or complex or conjugate binds to (or is recognised by) T cells in said sample, or whether said sample contains antibodies which bind to (or recognise) said peptide or epitope or complex or conjugate, wherein the binding of said peptide or epitope or complex or conjugate to T cells (or the activation of T cells), or the presence of said antibodies in the sample (as determined, for example, by the binding of said antibodies to said peptide or epitope or complex or conjugate), indicates that the subject has, or is susceptible to, CeD.

Thus, such methods or diagnostic methods can also be used for determining or monitoring the progression of CeD in a subject, wherein the binding of said peptide or epitope or complex or conjugate to T cells or the presence of said antibodies in the sample, indicates that CeD is present (or still present). Typically such methods involve the analysis of samples at different time points and a result which shows that the binding of said peptide or epitope or complex or conjugate to T cells (or the activation of T cells) or the presence of said antibodies in the sample (as determined, for example, by the binding of said antibodies to said peptide or epitope or complex or conjugate) is reduced, preferably measurably or significantly reduced, compared to a previous result on the same subject at a previous time point indicates that the CeD is improving.

Similarly, such methods can be used for determining or monitoring the efficacy of a CeD therapy, e.g. a CeD therapeutic method of the invention or any other form of CeD therapy.

Thus, such methods or diagnostic methods can also be used for determining or monitoring the efficacy of CeD therapy in a subject, wherein the binding of said peptide or epitope or complex or conjugate to T cells (or the activation of T cells) or the presence of said antibodies in the sample (as determined, for example, by the binding of said antibodies to said peptide or epitope or complex or conjugate), indicates that CeD is present (or still present). Typically such methods involve the analysis of samples at different time points, for example, before and after treatment, and/or at several time points after treatment, and a result which shows that the binding of said peptide or epitope or complex or conjugate to T cells (or the activation of T cells) or the presence of said antibodies in the sample (as determined, for example, by the binding of said antibodies to said peptide or epitope or complex or conjugate) is reduced, preferably measurably or significantly reduced, compared to a previous result on the same subject at a previous time point indicates that the CeD is improving or being treated effectively.

Such methods or diagnostic methods can conveniently be carried out in vitro, although any appropriate methodology or assays can be used. Appropriate in vitro assays could be carried out, for example, by immobilising peptides or epitopes of the invention, or appropriate complexes or conjugates thereof, e.g. pMHC molecules, to a solid support and detecting binding of T cells or antibodies by appropriate techniques such as ELISA assays.

Equally such solid supports could be used to detect activation of T cells (see, for example, Frick et al., 2020, European J. Imm. 50(1):142-145). Measuring the activation of T cells is another convenient way of measuring binding of T cells to peptides or epitopes or complexes or conjugates of the invention. Appropriate methods to measure such activation would be well known to a person skilled in the art and any of these may be used.

In one embodiment, the invention provides a method of diagnosing CeD in a subject comprising the step of:
(a) contacting a test sample taken from said subject with one or more of the peptides or epitopes or complexes or conjugates of the invention.

In a further embodiment, the invention provides a method of diagnosing CeD in a subject comprising the steps of:
(a) contacting a test sample taken from said subject with one or more of the peptides or epitopes or complexes or conjugates of the invention;
(b) measuring the presence and/or amount of T cells or antibodies in the test sample that bind to (or recognize) said peptides or epitopes or complexes or conjugates; and, optionally
(c) comparing the presence and/or amount of T cells or antibodies in the test sample to a control.

In the above methods, said contacting step is carried out under conditions that permit the formation (e.g. detectable formation) of a T cell-peptide/epitope/complex/conjugate or an antibody-peptide/epitope/complex/conjugate complex. Appropriate conditions can readily be determined by a person skilled in the art.

In the above methods any appropriate test sample or biological sample may be used, for example, a blood or serum sample, biopsy cells, material from tissues or organs suspected of being affected by CeD (e.g. small intestine) or histological sections.

In certain of the above methods, the presence in the test sample of any amount of a T cell-peptide/epitope/complex/conjugate or an antibody-peptide/epitope/complex/conjugate complex would be indicative of the presence of CeD. Preferably, for a positive diagnosis to be made, the amount of a T cell-peptide/epitope/complex/conjugate or an antibody-peptide/epitope/complex/conjugate complex in the test sample is greater than, preferably measurably or significantly greater than, the amount found in an appropriate control sample (a control value or level). More preferably, the significantly greater levels are statistically significant, preferably with a probability value of <0.05. Appropriate methods of determining statistical significance are well known and documented in the art and any of these may be used.

Appropriate control samples could be readily chosen by a person skilled in the art. For example, in the case of diagnosis of CeD, an appropriate control would be a sample from a subject that did not have CeD, e.g. a healthy subject. Appropriate control “values” or “levels” could also be readily determined without running a control “sample” in every test, e.g. by reference to the range for normal or healthy subjects known in the art. The control value or level may thus correspond to the level in appropriate control subjects or samples, e.g. may correspond to a cut-off level or range found in a control or reference population. Alternatively, said control value or level may correspond to the level in the same individual subject, or a sample from said subject, measured at an earlier time point (e.g. comparison with a “baseline” level in that subject). This type of control level (i.e. a control level from an individual subject) is particularly useful for embodiments of the invention where serial or periodic measurements in individuals, either healthy or ill, are taken looking for changes in the levels, e.g. in embodiments involving monitoring of subjects. In this regard, an appropriate control value or level can be the individual's own baseline, stable, nil, or previous level (as appropriate) as opposed to a control or cutoff level found in a general control (e.g. healthy) population. Control levels may also be referred to as “normal” levels or “reference” levels. The control level may be a discrete figure or a range.

Although the control value or level for comparison could be derived by testing an appropriate set of control subjects, the methods of the invention would not necessarily involve carrying out active tests on control subjects as part of the methods of the present invention but would generally involve a comparison with a control level which had been determined previously from control subjects and was known to the person carrying out the methods of the invention.

In one embodiment the method of diagnosing celiac disease is an in vitro method.

In one embodiment the method of diagnosing celiac disease is an in vivo method.

Alternatively viewed, the present invention provides a method for screening for celiac disease in a subject.

In some embodiments, the epitopes or peptides or complexes or conjugates of the present invention can be used as companion diagnostics.

The present invention further provides a method of detecting or determining or measuring the presence or amounts (levels) of T cells or antibodies that bind to the peptides or epitopes or complexes or conjugates of the invention, in a sample from a subject. For example, said method comprises contacting a sample from the subject with a peptide or epitope or complex or conjugate of the invention and determining whether said peptide or epitope or complex or conjugate binds to (or is recognised by) T cells in said sample, or whether said sample contains antibodies which bind to (or recognise) said peptide or epitope or complex or conjugate.

Alternatively viewed, the present invention provides a method of analysing for the presence or absence or level or amount of T cells or antibodies that bind to the peptides or epitopes or complexes or conjugates of the invention, in a sample from a subject. For example, said method comprises contacting a sample from the subject with a peptide or epitope or complex or conjugate of the invention and determining whether said peptide or epitope or complex or conjugate binds to (or is recognised by) T cells in said sample, or whether said sample contains antibodies which bind to (or recognise) said peptide or epitope or complex or conjugate.

The present invention further provides a method for detecting or determining or measuring the presence, absence, amount (or level) of a peptide or epitope or complex or conjugate of the invention in a sample. Such methods may comprise, for example, detecting etc., whether a sample, e.g. a sample from a subject, e.g. a biological sample, contains a peptide or epitope or complex or conjugate of the invention, or the amount (or level) of said peptide or epitope.

The features and discussion herein in relation to the methods of diagnosis for CeD can be applied, mutatis mutandis, to the above methods of detecting, etc., of the present invention.

In the above embodiments relating to diagnosis, monitoring, determining, analysing, screening, or detection, the epitopes or peptides or complexes or conjugates of the invention can be provided in any appropriate format suitable for binding to T cells or antibodies, for example, a format suitable to allow measurable or detectable binding. Thus, said peptides or epitopes can be provided as naked, free, or isolated peptides. However, it can also be helpful for the epitopes or peptides to be provided as a complex or conjugate (e.g. as an isolated or synthetic or recombinantly produced complex or conjugate) with one or more MHC molecules, e.g. provided as pMHC complexes or conjugates, or as larger complexes with multiple pMHC complexes present, e.g. multimers of pMHC, e.g. tetramers. Appropriate pMHC containing complexes are described elsewhere herein. As described elsewhere herein, the peptides or complexes can conveniently be attached to a solid support or other carrier, or, for example, can be expressed on the surface of the cell, e.g. as a pMHC, or loaded onto the surface of a cell, e.g. a cell already expressing appropriate MHC molecules can be loaded with peptides or epitopes of the invention.

In the above embodiments, binding of the peptides or epitopes or complexes or conjugates to T cells also includes binding of the peptides or epitopes or complexes or conjugates to TCRs, for example, either on the surface of T cells or in a soluble or recombinant format. Activation of T cells can be measured (e.g. by monitoring appropriate markers such as IL-2 levels, intracellular IFN-gamma, e.g. using flow cytometry, e.g. as described in the Examples, or by monitoring other appropriate cytokine levels, e.g. using flow cytometry, or by assessing for the presence/up-regulation of CD69, for example, CD69 upregulation on a CD19 negative population, e.g. using flow cytometry, e.g. as described in the Examples) as a way of detecting binding.

In the above embodiments relating to diagnosis or detection, the epitopes or peptides or complexes or conjugates of the invention are typically deamidated versions (or E residue containing versions), as these are the epitopes typically associated with CeD.

In the above embodiments relating to diagnosis or detection (or any other appropriate embodiments described herein), the epitopes or peptides or complexes or conjugates of the invention can be labelled, e.g. with a detectable label, or otherwise modified, in other words can be labelled or modified epitopes or peptides or complexes or conjugates. Binding proteins of the invention and as described herein can also be labelled, e.g. with a detectable label, in other words can be labelled binding proteins.

The present invention further provides a method for determining whether a composition, for example, a foodstuff or other ingestible material, is capable of causing CeD, said method comprising the step of detecting the presence of a peptide or epitope of the invention in said composition. Presence of a peptide or epitope of the invention, in particular at a measurable or significant level, is indicative that the composition is capable of causing CeD.

A further aspect of the present invention provides the peptides or epitopes or complexes or conjugates of the invention defined herein (or nucleic acid molecules encoding said peptides or epitopes or complexes or conjugates) for use in therapy. For example, epitope-specific immunotherapy is a form of antigen-specific immunotherapy that uses peptides instead of whole antigen to target and modify CD4+ T cells.

By “therapy” as used herein is meant the treatment of any medical condition. Treatment of disease or conditions in accordance with the present invention (for example treatment of pre-existing disease) includes cure of said disease or conditions, or any reduction or alleviation of disease (e.g. reduction in disease severity) or symptoms of disease. The therapeutic methods and uses of the prevent invention are suitable for prevention of disease as well as active treatment of disease (for example treatment of pre-existing disease). Thus, such treatment may be prophylactic (i.e. preventative), curative (or treatment intended to be curative), or palliative (i.e. treatment designed merely to limit, relieve or improve the symptoms of a condition).

Preferably, peptides or epitopes or complexes or conjugates of the invention defined herein are for use in the treatment or prevention of CeD.

Thus, in one aspect, the present invention provides the peptides or epitopes of the invention defined herein (e.g. free or isolated peptides or epitopes of the invention) for use in the treatment or prevention of CeD.

In another aspect, the present invention provides the conjugates or complexes of the invention, in particular conjugates or complexes of the epitopes or peptides of the invention with MHC molecules (pMHC), for use in therapy, in particular for use in the treatment or prevention of CeD.

In the embodiments described herein, nucleic acid molecules encoding said peptides or epitopes or conjugates or complexes may equally be used for therapy.

The in vivo methods and uses as described herein e.g. the therapeutic uses, are generally carried out in a human.

Thus, the term “animal” or “patient” or “subject” as used herein typically means human.

The therapeutic methods and uses of the invention can take any appropriate form, some of which are discussed below. In addition, any appropriate formulation (pharmaceutical formulation) of the peptides or epitopes or conjugates or complexes of the invention (or nucleic acid molecules encoding said peptides or epitopes or complexes or conjugates) can be used, examples of which are described elsewhere herein.

The therapeutic methods and uses of the invention can take the form of vaccination or tolerizing therapies.

For example, the peptides of the invention, either as free or isolated peptides or when associated with MHC molecules (pMHC), can be used to tolerize a subject to a gluten or gliadin protein, for example, to suppress or reduce the immune response, e.g. to reduce the production of a T cell response (e.g. a CD4 and/or CD8 T cell response) or an antibody (B cell) response to said peptide. Thus, a further aspect provides the peptides, epitopes, conjugates or complexes of the invention (or nucleic acid molecules encoding said peptides or epitopes or complexes or conjugates) for use in the tolerization of a subject to a gluten or gliadin peptide, or for use to suppress or reduce an immune response.

Such tolerization or suppression methods can then be used to treat or prevent CeD.

Tolerization leads to a decrease in the recognition of an epitope or peptide of the invention by the immune system, e.g. by T cells and/or B cells that recognise the epitope or peptide. Thus, after such tolerization, T-cell activity in response to the epitope is decreased or the T cells become unresponsive (anergy). Alternatively, or in addition, after such tolerization, decreased amounts of antibodies to the epitope are produced when the epitope is present. Such tolerization can also involve the production or induction of Treg cells (e.g. antigen/epitope specific or gluten specific Treg cells) which further suppress the immune response.

In such uses, the peptides and epitopes of the invention are presented to the immune system in a tolerizing context, for example, such epitopes can promote the generation and expansion of antigen (epitope) specific Tregs (gluten-specific Tregs) to induce immune tolerance. Methods of presenting antigens (e.g. the peptides or epitopes of the invention) to the immune system in such a context are known and described in the art. In this regard, the peptides or epitopes of the invention can be in the form of free or isolated peptides (e.g. as peptides per se, see for example Goel et al., Lancet Gastroenterol. Hepatol., 2017, 2(7):479-493) or for example as pMHC molecules (see for example Clemente-Casares et al., 2002, supra).

As the peptides and epitopes of the invention are derived from (or are highly similar to) gluten proteins, are associated (or are highly similar to peptides associated) with CeD, are able to bind to HLA-DQ2.5 and/or HLA-DQ2.2, and they (or substantially homologous peptides or epitopes) can be recognised by gluten-specific or gluten-reactive CD4+ T cells, such peptides or epitopes or complexes or conjugates of the invention can be used to engage such T cells and render them less responsive or unresponsive to further antigenic stimulation.

In such methods, for example, the peptides or epitopes or complexes or conjugates can be administered at various doses, for example, at a predetermined maximum tolerated dose, or at gradually increasing doses, to CeD patients on a gluten-free diet, after which they are challenged with gluten. Initial administrations may result in symptoms similar to an oral gluten challenge. However, in later or subsequent administrations, eventually such symptoms should not be present, for example, only placebo like symptoms or no symptoms would be observed.

In such methods the gluten-specific CD4+ T cells are driven into anergy, which means that they cease to be responsive to antigen or are unresponsive to antigen, and, for example, do not produce or produce significantly reduced levels of inflammatory cytokines such as interferon-γ.

For such methods, a peptide or epitope or complex or conjugate of the invention can be used alone or in combination with other T-cell epitopes or peptides, e.g. other peptides or epitopes of the invention (e.g. a mixture of such peptides, e.g. a mixture of 2, 3, 4 or 5 such peptides) or with different T cell epitopes or peptides as known and described in the art, in particular other T cell epitopes or peptides that are associated with or specific to CeD (e.g. those described in Sollid et al., Immunogenetics, 72 (1-2):85-88). Such T cell epitopes or peptides have the capacity (or capability) to engage with (or bind to, or activate) CeD-specific T cells.

Such compositions comprising a peptide or epitope or complex or conjugate of the invention can be regarded as tolerizing vaccines or vaccine compositions and can be used to treat or prevent CeD. Thus, compositions, e.g. vaccine compositions, comprising a peptide or epitope or complex or conjugate of the invention form a yet further aspect of the invention.

Other tolerizing therapies can involve the use of nanoparticles or other types of nanoformulations (see, for example, Clemente-Casares et al., 2016 Nature 530, 434-4402; Freitag et al., 2020, Gastroenterology 158(6):1667-1681). For example, nanoparticles can be coated or associated with peptides or epitopes of the invention either alone, e.g. as isolated, free or uncomplexed peptides, or in association with an MHC molecule, in particular an MHC class II molecule. In other words, the nanoparticles are coated or associated with peptides or epitopes of the invention or with pMHC molecules in which the peptide (p) is a peptide or epitope of the invention and the MHC molecule is one which is capable of binding to said peptide or epitope. Typically, therefore in the present invention such MHC molecules are HLA-DQ2 molecules, in particular HLA-DQ2.5 or HLA-DQ2.2. Such description of pMHC molecules is appropriate for other aspects and embodiments of the invention as described elsewhere herein.

The administration of such nanoparticles can promote the generation and expansion of antigen-specific Tregs (for example TR1 like cells) which can, for example, then act to suppress auto-antigen loaded APCs, in particular APCs in which the MHC class II molecules are loaded with or presenting the epitopes or peptides of the invention. Thus, said nanoparticles can act to suppress the immune response.

Thus, such nanoparticles coated or associated with peptides or epitopes of the invention either alone or in association with an MHC molecule, in particular an MHC class II molecule or nanoparticles coated with peptides or epitopes of the invention or with pMHC in which the peptide (p) is a peptide or epitope of the invention and the MHC molecule is one which is capable of binding to said peptide or epitope as described above, form yet preferred aspects of the invention.

Other types of formulations, e.g. pharmaceutical formulations or pharmaceutical carriers, e.g. nanoformulations, can equally be used in place of nanoparticles, for example, lipid-based formulations such as liposomes or micelles. Such formulations can be loaded in the interior or core with peptides or epitopes of the invention, for example, with free or isolated peptides or epitopes, or with pMHC complexes as described above and elsewhere herein. Equally, said peptides or epitopes of the invention or pMHC complexes can be associated with or conjugated to the exterior surface of the lipid structures. Other entities to promote tolerance may also be included in the lipid formulations (or indeed in the other formulations of the invention). As is known to the skilled person, a micelle is an aggregate of surfactants (e.g. fatty acids) in an aqueous liquid, in which the hydrophilic head groups of the surfactants form the surface of the aggregate and the hydrophobic tail groups the core. A liposome is a spherical vesicle formed from a lipid bilayer surrounding an aqueous core.

Liposomes and micelles may be synthesised using any method known in the art. Suitable methods for liposome synthesis and drug loading are described in e.g. Akbarzadeh et al., Nanoscale Res Lett 8(1): 102, 2013. Liposomes and micelles may be conjugated to appropriate proteins using methods known in the art, e.g. the methods taught in Reulen et al., Bioconjug Chem 18(2): 590-596, 2007; or Kung & Redemann, Biochim Biophys Acta 862(2): 435-439, 1986.

In general, where peptides of the invention are used in the pMHC format, formats comprising multiple pMHC units (multimers) can be made and are sometimes preferred. For example, dimeric or tetrameric pMHC formats are known in the art and can conveniently be used, for example, in detection, diagnosis and therapeutic applications.

Thus, a yet further aspect of the invention provides the use of a peptide or epitope or complex or conjugate of the invention (or a nucleic acid molecule encoding said peptide or epitope or complex or conjugate) in the manufacture of a medicament or composition for use in therapy, preferably the treatment or prevention of CeD.

A yet further aspect of the invention provides the use of a peptide or epitope or complex or conjugate of the invention (or a nucleic acid molecule encoding said peptide or epitope or complex or conjugate) in the manufacture of a medicament or composition for vaccination or tolerizing therapy, e.g. for use in the tolerization of a subject to said peptide or epitope or complex or conjugate, or for use to suppress or reduce an immune response to said peptide or epitope or complex or conjugate.

A yet further aspect of the invention provides a method of treating or preventing CeD in a subject, said method comprising the step of administrating an effective amount of a peptide or epitope or complex or conjugate of the invention (or a nucleic acid molecule encoding said peptide or epitope or complex or conjugate) to said subject.

A yet further aspect of the invention provides a method of vaccination or tolerizing therapy in a subject, said method comprising the step of administrating an effective amount of a peptide or epitope or complex or conjugate of the invention (or a nucleic acid molecule encoding said peptide or epitope or complex or conjugate) to said subject.

A yet further aspect of the invention provides a method of tolerization of a subject to a peptide or epitope or complex or conjugate of the invention, said method comprising the step of administrating to said subject an effective amount of a peptide or epitope or complex or conjugate of the invention (or a nucleic acid molecule encoding said peptide or epitope or complex or conjugate) to said subject.

A yet further aspect of the invention provides a method of suppressing or reducing an immune response in a subject to a peptide or epitope or complex or conjugate of the invention, said method comprising the step of administrating to said subject an effective amount of a peptide or epitope or complex or conjugate of the invention (or a nucleic acid molecule encoding said peptide or epitope or complex or conjugate) to said subject.

Said methods or uses preferably involve the administration of said peptide or epitope or complex or conjugate of the invention (or a nucleic acid molecule encoding said peptide or epitope or complex or conjugate) in pharmaceutically or physiologically or therapeutically effective amounts, to a subject in need of same.

By “pharmaceutically or physiologically or therapeutically effective amount” is meant an amount sufficient to show benefit to the condition of the subject or to show the relevant physiological effect in the subject, e.g. tolerization or reduction in the immune response. Whether an amount is sufficient to show benefit to the condition of the subject or a relevant physiological effect in a subject may be determined by the subject him/herself or a physician.

Alternative and preferred embodiments and features of the invention as described elsewhere herein apply equally to these methods of treatment and uses of the invention.

In some embodiments (e.g. methods of detection, diagnosing or therapeutic methods), the subject (e.g. a human subject) is a subject at risk of developing CeD or at risk of the occurrence of CeD, e.g. a healthy subject or a subject not displaying any symptoms of celiac disease or any other appropriate “at risk” subject, e.g. first degree relatives of patients with CeD, or those with associated high risk disorders such as type I diabetes, selective IgA deficiency, autoimmune thyroiditis, Sjogren syndrome, Down syndrome, Addison disease, Turner syndrome, or Williams syndrome. In other embodiments, the subject (e.g. a human subject) is a subject having, or suspected of having (or developing), or potentially having (or developing) CeD.

In some embodiments, appropriate subjects (e.g. for detection, diagnosis or therapy) are those which are HLA-DQ 2.5 or HLA-DQ 2.2 positive subjects.

In some aspects, a method (e.g. a detection, diagnostic or therapeutic method) of the invention may further comprise an initial step of selecting a subject (e.g. a human subject), for example, a subject at risk of developing CeD, or at risk of the occurrence of celiac disease, or having celiac disease, or suspected of having (or developing) celiac disease, or potentially having (or developing) CeD. Subjects may be selected on the basis that, for example, the subject (or sample, e.g. tissue biopsy, or blood/serum sample from the subject) is positive for one or more CeD markers or risk factors.

In some aspects, diagnostic (or similar) methods of the invention are provided which further comprise a step of treating CeD by therapy, e.g. using a peptide or epitope of the present invention (or by using different T cell epitopes or peptides, in particular alternative T cell epitopes or peptides that are associated with or specific to CeD), for example, using therapeutic methods as described herein, or any other appropriate therapeutic method. For example, if the result of a diagnostic (or similar) method of the invention is indicative of CeD in the subject (e.g. a positive diagnosis of CeD is made), then an additional step of treating the CeD by therapy can be performed. Appropriate therapeutic methods are described in the art and include gluten-free diet (GFD) or antibody therapy, e.g. with anti-CD20 antibodies such as rituximab.

In other aspects, the diagnostic (or similar) method of the invention can be used in conjunction with other additional diagnostic methods appropriate for CeD. Any such additional method may be used, for example, those described in the art such as CeD serology tests, including measuring or the assessment of TG2 antibodies, or measuring or the assessment of antibodies for other gluten peptides associated with CeD, or the use of small intestine histology techniques or biopsy.

In other aspects the therapeutic methods of the invention can be used in conjunction with other additional therapeutic methods appropriate for CeD. Any such additional method may be used, for example, those described in the art such as gluten-free diet (GFD)) or antibody therapy, e.g. with anti-CD20 antibodies such as rituximab, or therapy by using different T cell epitopes or peptides, in particular alternative T cell epitopes or peptides that are associated with or specific to CeD (as described elsewhere herein). In some embodiments the therapeutic methods of the invention can be used as rescue therapy after incidental gluten exposure.

The invention further includes kits comprising one or more of the peptides, epitopes, complexes, conjugates (e.g. pMHC conjugates), vaccines, binding proteins, or compositions of the invention or one or more of the nucleic acid molecules encoding such entities.

Preferably said kits are for use in the methods and uses as described herein, e.g. the therapeutic or detection or diagnostic methods as described herein, or are for use in the in vitro assays or methods as described herein. Preferably said kits comprise appropriate instructions for use of the kit components in accordance with the invention. Preferably said kits are for treating or diagnosis of diseases as described elsewhere herein, or for detection methods, and optionally comprise instructions for use of the kit components to treat or diagnose such diseases, or for detection.

The peptides or epitopes or complexes or conjugates of the invention as defined herein may also be used as molecular tools for in vitro or in vivo applications and assays.

Thus, yet further aspects of the invention provide a reagent that comprises a peptide or epitope or complex or conjugate of the invention as defined herein and the use of such peptides or epitopes or complexes or conjugates as molecular tools, for example, in in vitro or in vivo assays. Particularly preferred molecular tools and reagents may comprise or consist of peptides or epitopes of the invention associated with MHC molecules (pMHC molecules) as described herein, which may be in the form of multimers, e.g. in the form of dimers or tetramers.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “epitope”, or a “peptide”, etc., as used herein, means “at least a first epitope” or “at least a first peptide”. The operable limits and parameters of combinations, as with the amounts of any single agent, will be known to those of ordinary skill in the art in light of the present disclosure.

In addition, where the terms “comprise”, “comprises”, “has” or “having”, or other equivalent terms are used herein, then in some more specific embodiments these terms include the term “consists of” or “consists essentially of”, or other equivalent terms. Methods comprising certain steps also include, where appropriate, methods consisting of these steps.

The epitopes, peptides, complexes, conjugates, binding proteins, nucleic acid molecules, and cells, e.g. APCs, of the invention are generally “isolated” or “purified” molecules insofar as they are distinguished from any such components that may be present in situ within a human or animal body or a tissue sample derived from a human or animal body. The sequences may, however, correspond to or be substantially homologous to sequences as found in a human or animal body. Thus, the term “isolated” or “purified” as used herein in reference to nucleic acid molecules or sequences and proteins, peptides or polypeptides, e.g. epitopes, refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

The term “increase” or “enhance” (or equivalent terms) as described herein includes any measurable increase or elevation when compared with an appropriate control. Appropriate controls would readily be identified by a person skilled in the art and appropriate examples are described herein. Preferably the increase will be significant, for example, clinically or statistically significant, for example, with a probability value of 50.05, when compared to an appropriate control level or value.

The term “decrease” or “reduce” (or equivalent terms) as described herein includes any measurable decrease or reduction when compared with an appropriate control. Appropriate controls would readily be identified by a person skilled in the art and appropriate examples are described herein. Preferably the decrease will be significant, for example, clinically or statistically significant, for example, with a probability value of 50.05, when compared to an appropriate control level or value.

Methods of determining the statistical significance of differences between test groups of subjects or differences in levels or values of a particular parameter are well known and documented in the art. For example, herein a decrease or increase is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of 50.05.

List of Some Nucleotide and Amino Acid Sequences Disclosed Herein and their Sequence Identifiers (Seq Id Nos)

All nucleotide sequences are recited herein 5′ to 3′ in line with convention in this technical field. All amino acid sequences are recited herein from the N-terminus to the C-terminus in line with convention in this technical field.

### Amino Acid Sequence of Tritium Urartu Omega Aliadin (SEQ ID NO:1)

UPI000618D06A (A0A0E3SZN6)
Omega-gliadin Triticum urartu
MKTFLIFVLLAMAMNIATAARQLNPSNKELQSPQQSFSHQQQPFLQEPYP
QQPYPSQQPYPSQQPFPTPQQQFSQQSQQPFPQTQQSFPLQPQQPFPQQP
QQPFPQPQLPFPQQPEQIIPQQPQQPFPLQPQQPFPQQPQQPFPQPQQPI
SVQPQQPFPQQSQQSQQPFPQPQQLFLELQQPIHQQPQQPFPQQPQQPFP
QQPQQPFPQQPQQPFPLQPQQPFPQQPQQSFLLGPQQPFPQQPQQSQQSF
PQPQPQQPQQPSIMQPQQPLPQRPQQPFLLPQQQLSQQPEQTISQQPQQP
HQPQQPYPQQQQPYGTSLTSIGGQ

Mature α-chain of HLA-DQ2.5 MHC molecule (SEQ ID NO:2) (IMGT-HLA allele name: DQA1*05:01:01:01)

IVADHVASYGVNLYQSYGPSGQYTHEFDGDEQFYVDLGRKETVWCLPVLR
QFRFDPQFALTNIAVLKHNLNSLIKRSNSTAATNEVPEVTVFSKSPVTLG
QPNILICLVDNIFPPVVNITWLSNGHSVTEGVSETSFLSKSDHSFFKISY
LTLLPSAEESYDCKVEHWGLDKPLLKHWEPEIPAPMSELTETVVCALGLS
VGLVGIVVGTVFIIRGLRSVGASRHQGPL

Mature β-chain of HLA-DQ2.5 MHC molecule (SEQ ID NO:3) (IMGT-HLA allele name:DQB1*02:01:01)

RDSPEDFVYQFKGMCYFTNGTERVRLVSRSIYNREEIVRFDSDVGEFRAV
TLLGLPAAEYWNSQKDILERKRAAVDRVCRHNYQLELRTTLQRRVEPTVT
ISPSRTEALNHHNLLVCSVTDFYPAQIKVRWFRNDQEETAGVVSTPLIRN
GDWTFQILVMLEMTPQRGDVYTCHVEHPSLQSPITVEWRAQSESAQSKML
SGIGGFVLGLIFLGLGLIIHHRSQKGLLH

TABLE 1
107 Antibody

| SEQ ID NO: | Description | Sequence |
|---|---|---|
| 66 | VH domain (nt) | IGHV6-1*01/ IGHJ6*02/IGHD6-19*01 |
| | | CAGGTACAGCTGCAGCAGTCAGGTCCAGGA |
| | | CTGGTGAAGCCCTCGCAGACCCTCTCACTC |
| | | ACCTGTGCCATCTCCGGGGACAGTGTCTCT |
| | | AGCAACAGTGCTGCTTGGAACTGGATCAGG |
| | | CAGTCCCCATCGAGAGGCCTTGAGTGGCTG |
| | | GGAAGGACATACTACAGGTCCAAGTGGTAT |
| | | AATGATTATGCAGTATCTGTGAAAAGTCGAA |
| | | TAACCATCAACCCAGACACATCCAAGAACCA |
| | | GTTCTCCCTGCAGCTGAACTCTGTGACTCCC |
| | | GAGGACACGGCTGTGTATTACTGTGCAAGA |
| | | GATAGCAGCAGTGGCTGGCATCCTTACGGT |
| | | ATGGACGTCTGGGGCCAAGGGACCACGGTC |
| | | ACCGTCTCCTCA |
| 67 | VL domain (nt) | IGKV1-9*01/IGKJ5*01 |
| | | GACATCCAGGTGACCCAGTCTCCATCCTTCC |
| | | TGTCTGCATCTGTAGGAGACAGAGTCACCAT |
| | | CACTTGCCGGGCCAGTCACGACATTAGCAG |
| | | TTATTTAGCCTGGTATCAACACAAACCATGG |
| | | AAAGCCCCCAAACTCCTGATCCATGCTGCAT |
| | | CCATTTTGCAAAGTGGGGTCCCATCAAGGTT |
| | | CAGCGGAAGTGGATCTGGGACAGAATTCAC |
| | | TCTCACAATCAGCAGCCTGCAGCCTGAAGAT |
| | | TTTGCAACGTACTACTGTCAAGATCTCAATA |
| | | GTTATCCTCTCTTCGGCCAAGGGACACGACT |
| | | GGAGATTAAA |
| 68 | VH domain (aa) | QVQLQQSGPGLVKPSQTLSLTCAISGDSVSS |
| | | NSAAWNWIRQSPSRGLEWLGRTYYRSKWY |
| | | NDYAVSVKSRITINPDTSKNQFSLQLNSVTPE |
| | | DTAVYYCARDSSSGWHPYGMDVWGQGTTV |
| | | TVSS |
| 69 | VL domain (aa) | DIQVTQSPSFLSASVGDRVTITCRASHDISSYL |
| | | AWYQHKPWKAPKLLIHAASILQSGVPSRFSGS |
| | | GSGTEFTLTISSLQPEDFATYYCQDLNSYPLFG |
| | | QGTRLEIK |
| 40 | Heavy CDR1 | GDSVSSNSAA |
| 41 | Heavy CDR2 | TYYRSKWYN |
| 48 | Heavy CDR3 | ARDSSSGWHPYGMDV |
| 44 | Light CDR1 | HDISSY |
| 45 | Light CDR2 | AAS |
| 49 | Light CDR3 | QDLNSYPL |
| 70 | Heavy FR1 | QVQLQQSGPGLVKPSQTLSLTCAIS |
| 71 | Heavy FR2 | WNWIRQSPSRGLEWLGR |
| 72 | Heavy FR3 | DYAVSVKSRITINPDTSKNQFSLQLNSVTPEDT |
| | | AVYYC |
| 73 | Heavy FR4 | WGQGTTVTVSS |
| 74 | Light FR1 | DIQVTQSPSFLSASVGDRVTITCRAS |
| 75 | Light FR2 | LAWYQHKPWKAPKLLIH |
| 76 | Light FR3 | ILQSGVPSRFSGSGSGTEFTLTISSLQPEDFAT |
| | | YYC |
| 77 | Light FR4 | FGQGTRLEIK |
| 78 | Heavy chain (aa) | QVQLQQSGPGLVKPSQTLSLTCAISGDSVSSN |
| | (variable + constant | SAAWNWIRQSPSRGLEWLGRTYYRSKWYND |
| | domain). | YAVSVKSRITINPDTSKNQFSLQLNSVTPEDTA |
| | mIgG_{2b} | VYYCARDSSSGWHPYGMDVWGQGTTVTVSS |
| | | AKTTPPSVYPLAPGCGDTTGSSVTLGCLVKGY |
| | | FPESVTVTWNSGSLSSSVHTFPALLQSGLYTM |
| | | SSSVTVPSSTWPSQTVTCSVAHPASSTTVDKK |
| | | LEPSGPISTINPCPPCKECHKCPAPNLEGGPS |
| | | VFIFPPNIKDVLMISLTPKVTCVVVDVSEDDPD |
| | | VQISWFVNNVEVHTAQTQTHREDYASTIRVVS |
| | | TLPIQHQDWMSGKEFKCKVNNKDLPSPIERTI |
| | | SKIKGLVRAPQVYILPPPAEQLSRKDVSLTCLV |
| | | VGFNPGDISVEWTSNGHTEENYKDTAPVLDS |
| | | DGSYFIYSKLNMKTSKWEKTDSFSCNVRHEGL |
| | | KNYYLKKTISRSPGK |
| 79 | Light chain (aa) | DIQVTQSPSFLSASVGDRVTITCRASHDISSYL |
| | (variable + constant | AWYQHKPWKAPKLLIHAASILQSGVPSRFSGS |
| | domain). | GSGTEFTLTISSLQPEDFATYYCQDLNSYPLFG |
| | mIgG_{2b} | QGTRLEIKRADAAPTVSIFPPSSEQLTSGGASV |
| | | VCFLNNFYPKDINVKWKIDGSERQNGVLNSWT |
| | | DQDSKDSTYSMSSTLTLTKDEYERHNSYTCEA |
| | | THKTSTSPIVKSFNRNEC |
| 80 | Heavy chain (aa) | QVQLQQSGPGLVKPSQTLSLTCAISGDSVSSN |
| | (variable + | SAAWNWIRQSPSRGLEWLGRTYYRSKWYND |
| | constant domain). | YAVSVKSRITINPDTSKNQFSLQLNSVTPEDTA |
| | hIgG₁ | VYYCARDSSSGWHPYGMDVWGQGTTVTVSS |
| | | ASTKGPSVFPLAPSSKSTSGGTAALGCLVKDY |
| | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPELLGGPSVFLFPP |
| | | KPKDTLMISRTPEVTCVVVDVSHEDPEVKFNW |
| | | YVDGVEVHNAKTKPREEQYGSTYRVVSVLTVL |
| | | HQDWLNGKEYKCKVSNKALPAPIEKTISKAKG |
| | | QPREPQVYTLPPSRDELTKNQVSLTCLVKGFY |
| | | PSDIAVEWESNGQPENNYKTTPPVLDSDGSFF |
| | | LYSKLTVDKSRWQQGNVFSCSVMHEALHNHY |
| | | TQKSLSLSPGK |
| 81 | Light chain (aa) | DIQVTQSPSFLSASVGDRVTITCRASHDISSYL |
| | (variable + | AWYQHKPWKAPKLLIHAASILQSGVPSRFSGS |
| | constant domain). | GSGTEFTLTISSLQPEDFATYYCQDLNSYPLFG |
| | hIgG₁ | QGTRLEIKRTVAAPSVFIFPPSDEQLKSGTASV |
| | | VCLLNNFYPREAKVQWKVDNALQSGNSQESV |
| | | TEQDSKDSTYSLSSTLTLSKADYEKHKVYACE |
| | | VTHQGLSSPVTKSFNRGEC |

TABLE 2
4.7C antibody

| SEQ ID NO: | Description | Sequence |
|---|---|---|
| 82 | VH domain (nt) | IGHV6-1*01 |
| | | IGHJ6*02 |
| | | IGHD6-19*01 |
| | | CAGGTACAGCTGCAGCAGTCAGGTCCAGGA |
| | | CTGGTGAAGCCCTCGCAGACCCTCTCACTCA |
| | | CCTGTGCCATCTCCGGGGACAGTGTCTCTAG |
| | | CAACAGTGCTGCTTGGAACTGGATCAGGCAG |
| | | TCCCCATCGAGAGGCCTTGAGTGGCTGGGA |
| | | AGGACATACTACAGGTCCAAGTGGTATAATG |
| | | ATTATGCAGTATCTGTGAAAAGTCGAATAACC |
| | | ATCAACCCAGACACATCCAAGAACCAGTTCT |
| | | CCCTGCAGCTGAACTCTGTGACTCCCGAGGA |
| | | CACGGCTGTGTATTACTGTGCAAGAGATAGG |
| | | ACTACTGGGTGGCATCCGTACGGTATGGACG |
| | | TCTGGGGCCAAGGGACCACGGTCACCGTCT |
| | | CCTCA |
| 83 | VL domain (nt) | IGKV1-9*01 |
| | | IGKJ5*01 |
| | | CAGGTACAGCTGCAGCAGTCAGGTCCAGGA |
| | | CTGGTGAAGCCCTCGCAGACCCTCTCACTCA |
| | | CCTGTGCCATCTCCGGGGACAGTGTCTCTAG |
| | | CAACAGTGCTGCTTGGAACTGGATCAGGCAG |
| | | TCCCCATCGAGAGGCCTTGAGTGGCTGGGA |
| | | AGGACATACTACAGGTCCAAGTGGTATAATG |
| | | ATTATGCAGTATCTGTGAAAAGTCGAATAACC |
| | | ATCAACCCAGACACATCCAAGAACCAGTTCT |
| | | CCCTGCAGCTGAACTCTGTGACTCCCGAGGA |
| | | CACGGCTGTGTATTACTGTGCAAGAGATTCG |
| | | ACTACGGGGTGGGGTGCGTACGGTATGGAC |
| | | GTCTGGGGCCAAGGGACCACGGTCACCGTC |
| | | TCCTCA |
| 84 | VH domain (aa) | QVQLQQSGPGLVKPSQTLSLTCAISGDSVSSN |
| | | SAAWNWIRQSPSRGLEWLGRTYYRSKWYNDY |
| | | AVSVKSRITINPDTSKNQFSLQLNSVTPEDTAVY |
| | | YCARDRTTGWHPYGMDVWGQGTTVTVSS |
| 69 | VL domain (aa) | DIQVTQSPSFLSASVGDRVTITCRASHDISSYLA |
| | | WYQHKPWKAPKLLIHAASILQSGVPSRFSGSG |
| | | SGTEFTLTISSLQPEDFATYYCQDLNSYPLFGQ |
| | | GTRLEIK |
| 40 | Heavy CDR1 | GDSVSSNSAA |
| 41 | Heavy CDR2 | TYYRSKWYN |
| 50 | Heavy CDR3 | ARDRTTGWHPYGMDV |
| 44 | Light CDR1 | HDISSY |
| 45 | Light CDR2 | AAS |
| 49 | Light CDR3 | QDLNSYPL |
| 70 | Heavy FR1 | QVQLQQSGPGLVKPSQTLSLTCAIS |
| 71 | Heavy FR2 | WNWIRQSPSRGLEWLGR |
| 72 | Heavy FR3 | DYAVSVKSRITINPDTSKNQFSLQLNSVTPEDT |
| | | AVYYC |
| 73 | Heavy FR4 | WGQGTTVTVSS |
| 74 | Light FR1 | DIQVTQSPSFLSASVGDRVTITCRAS |
| 75 | Light FR2 | LAWYQHKPWKAPKLLIH |
| 76 | Light FR3 | ILQSGVPSRFSGSGSGTEFTLTISSLQPEDFATY |
| | | YC |
| 77 | Light FR4 | FGQGTRLEIK |

The invention will now be further described in the following non-limiting Example with reference to the following drawings:

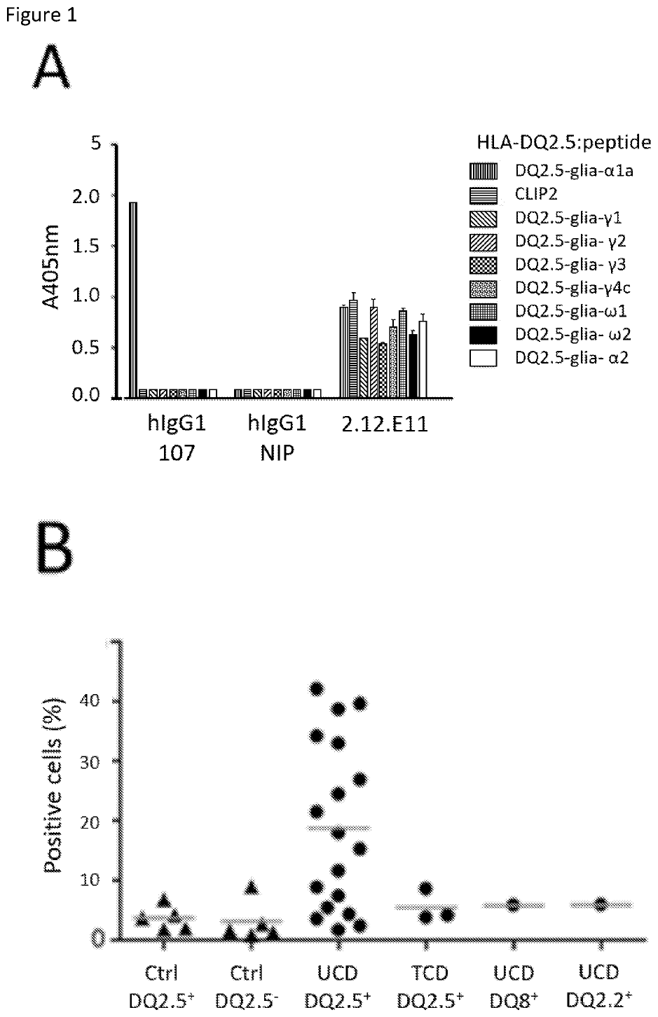

FIG. 1:

A. Binding properties of the HLA-DQ2.5:DQ2.5-glia-α1a-specific antibody 107. Eight different HLA-DQ2.5:gluten peptide complexes and HLA-DQ2.5:CLIP2 were used in ELISA for specificity analysis (n=2). mAb 2.12.E11 specific for the β-chain of HLA-DQ2 was included to control pMHC capture levels. Error bars illustrate mean±SD of duplicates. B. Plasma cells (PCs) and B cells of gut biopsies present the DQ2.5-glia-α1a peptide. Detection of DQ2.5-glia-α1a presentation among PCs and B cells in single-cell suspension prepared from intestinal biopsies from either untreated celiac disease (UCD) or treated celiac disease (TCD) patients or healthy controls. Mouse IgG2b mAb 107 was used for detection, and percentage of positive cells was determined relative to use of secondary antibody alone. Stratification of the control patients among the CD19+ PCs. Each symbol corresponds to one individual.

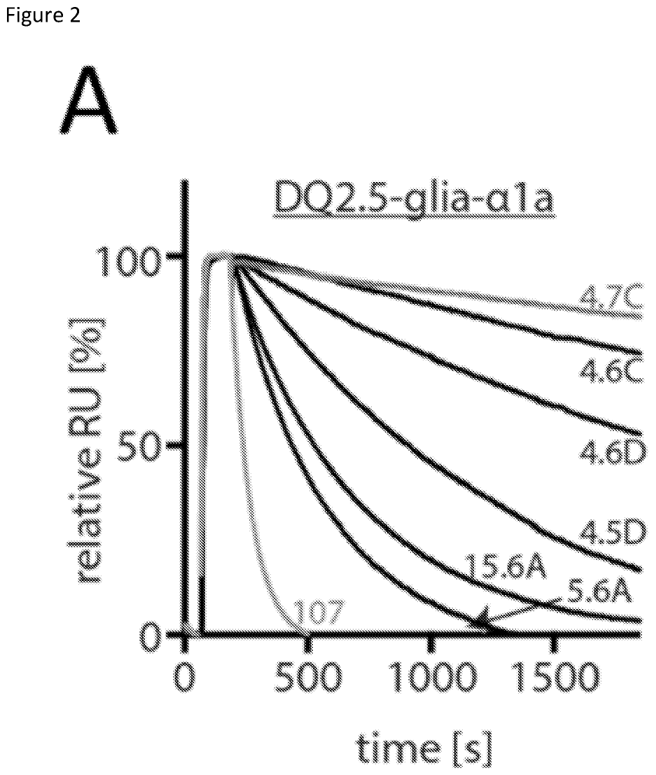

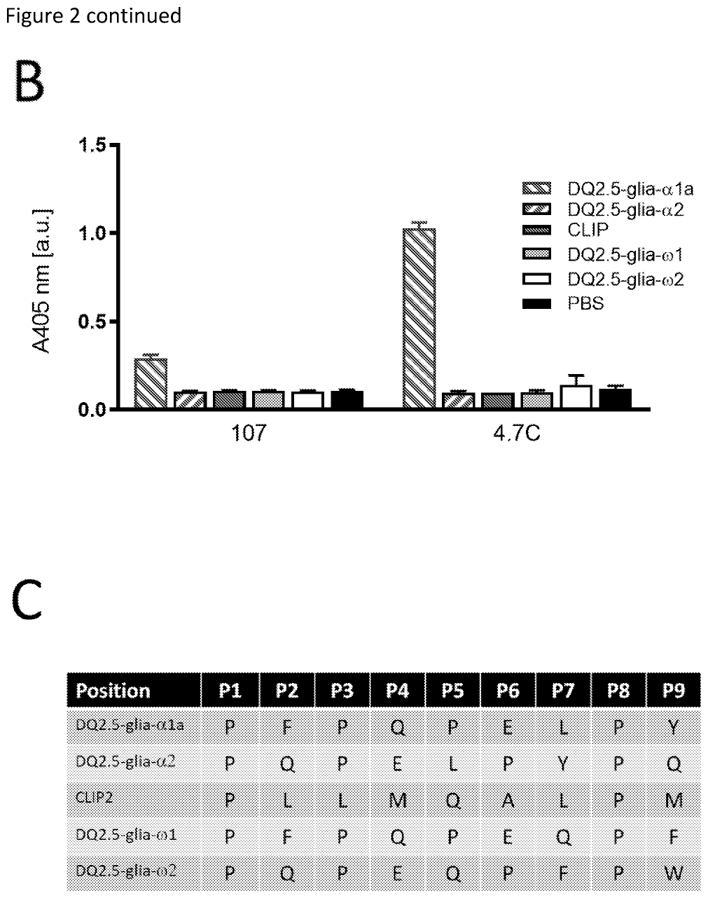

FIG. 2:

Biophysical characterization of leads. A. Fab fragments were ranked based on off-rate binding to HLA-DQ2.5:DQ2.5-glia-α1a using SPR. The individual clone IDs are indicated. B. The Fabs were reformatted to full-length hIgG1 and analyzed in ELISA against a panel of related soluble peptide:HLA-DQ2.5 complexes. Error bars illustrate mean±SD of duplicates. C. Sequence comparison of the 9-mer core epitopes of the respective peptides used in B.

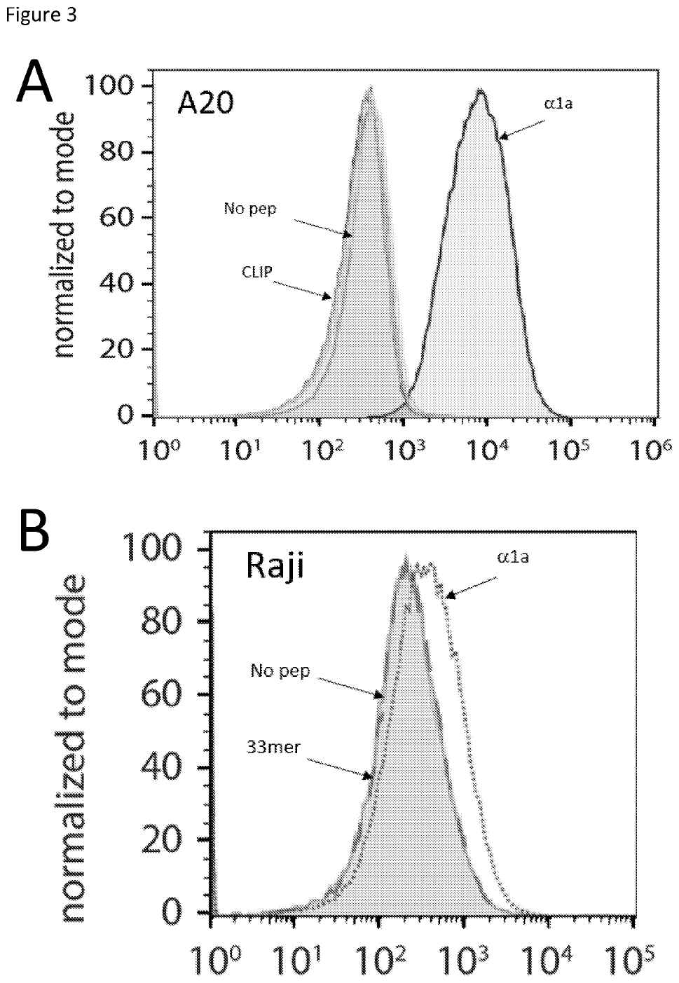

FIG. 3:

Assessment of mAb 4.7C binding to pMHC on cells. A. Murine A20 B cells engineered to express HLA-DQ2.5 with covalently linked peptide as indicated were stained with 5 μg/ml antibody. B: Raji B cells were in vitro loaded with 50 μM gluten peptides as annotated and stained with 5 μg/ml antibody (12-mer α1a peptide: QLQPFPQPELPY (SEQ ID NO:53); CLIP2 peptide (MATPLLMQALPMGAL (SEQ ID NO:54)): 33-mer: LQLQPFPQPELPYPQPELPYPQPELPYPQPQPF (SEQ ID NO:55)).

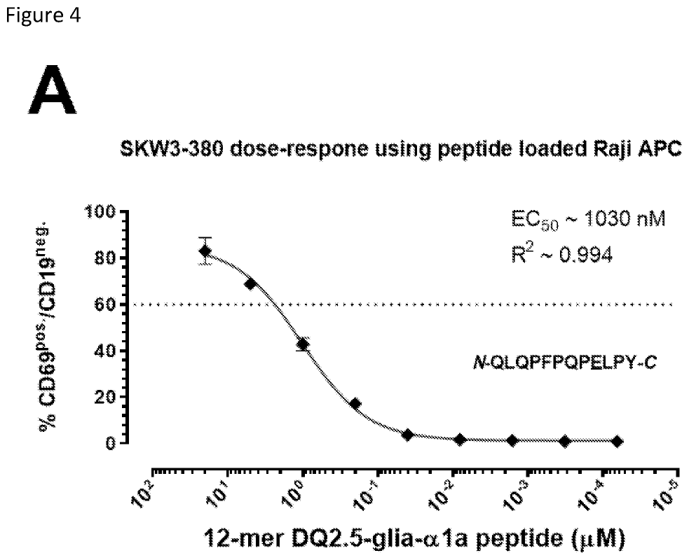

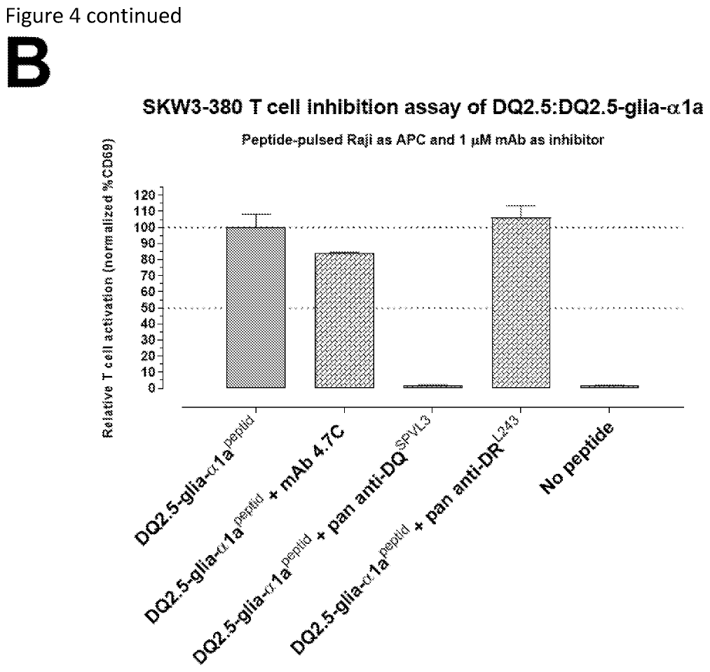

FIG. 4:

T cell activation inhibitory capacity of mAb 4.7C. A. Activation of gliadin-specific SKW3 T cells. Raji B cells were loaded with a serial dilution of peptide and co-cultured with engineered gliadin-specific SKW3 T cells. T-cell activation was measured as CD69+CD19-cells in flow cytometry. Error bars illustrate mean±SD of duplicates (n=2). B. At a peptide dose leading to 60% of maximum T-cell activation as measured by CD69 upregulation, 1 μM mAb 4.7C or 0.1 μM pan-HLA antibodies were added to the Raji B cells prior to incubation with T cells. T-cell activation was calculated relative to peptide-specific T-cell activation without presence of antibodies.

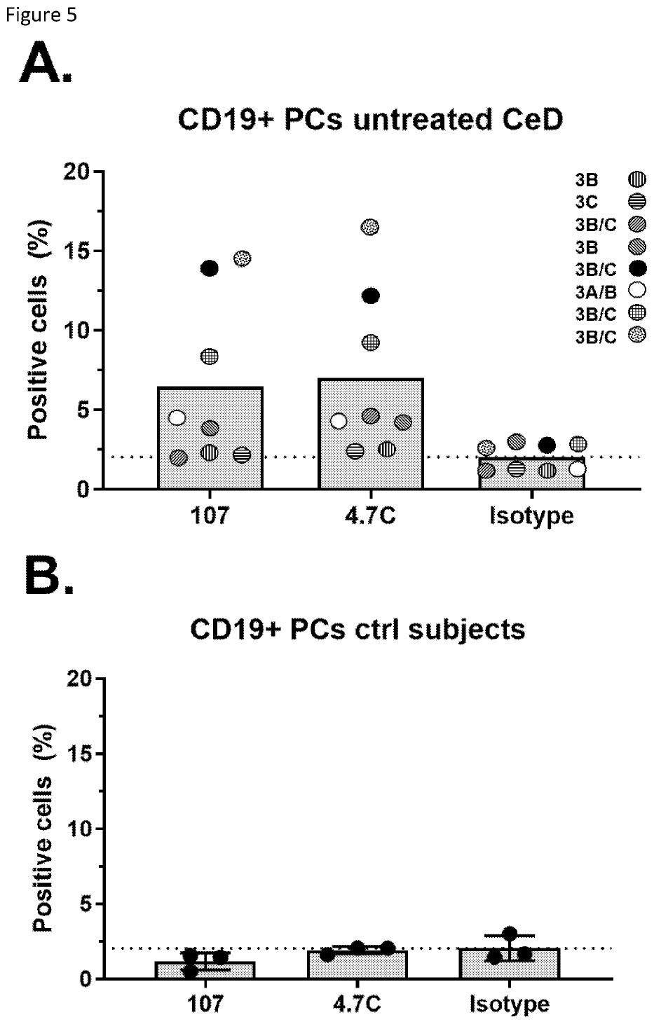

FIG. 5:

The pHLA-specific antibodies detect gluten peptide presentation on cells derived from small intestinal biopsies from CeD patients. Single-cell suspensions were prepared from either untreated HLA-DQ2.5+CeD patients (n=8) (A) or controls with a normal intestinal histology (n=3) (B). Cells were gated as live, large lymphocytes, CD3-CD11c-CD14-CD38+CD27+CD19+CD45+ PCs (A). Bound mlgG2b antibodies were detected with an Alexa-546-conjugated secondary antibody and the frequency of positive cells was calculated based on gates set according to the staining of an isotype control antibody (isotype). The mean percentage in each group is shown as horizontal lines and the dotted lines represent mean background staining of the isotype control. Each CeD patient is represented by a unique color and alterations in biopsy histology according to modified Marsh scores are indicated.

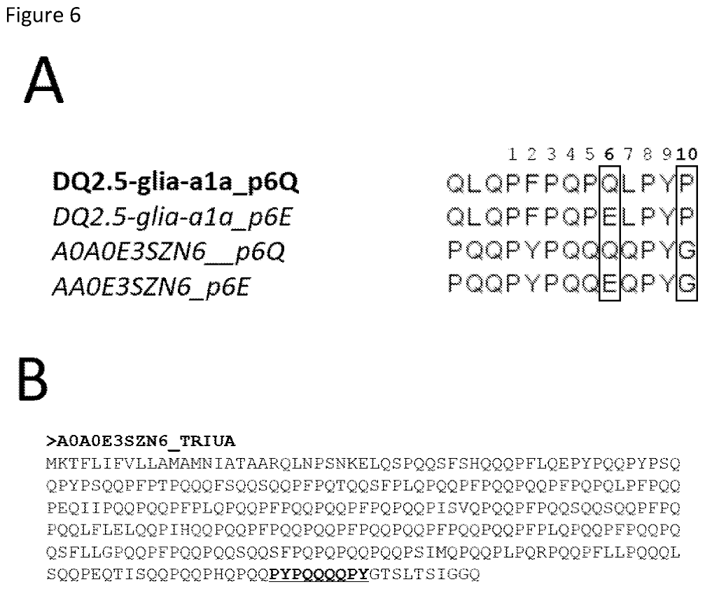

FIG. 6:

Identification of a Triticum urartu peptide homologous to DQ2.5-glia-α1a from Triticum aestivum. Using the ScanProsite search engine (https://prosite.expasy.org/scanprosite/) to search the Triticum taxa for DQ2.5-glia-α1a homologous peptides that did not have proline in p10, but preferably glycine, identified a peptide in the ancestral wheat species Triticum urartu annotated as Uniprot entry A0A0E3SZN6_TRIUA. Positions p6 and p10 are boxed for clarity (A). The complete amino acid sequence of Uniprot entry A0A0E3SZN6_TRIUA (B). The hypothetical 9-mer core of the DQ2.5-glia-α1a homologous peptide is highlighted in underlined bold.

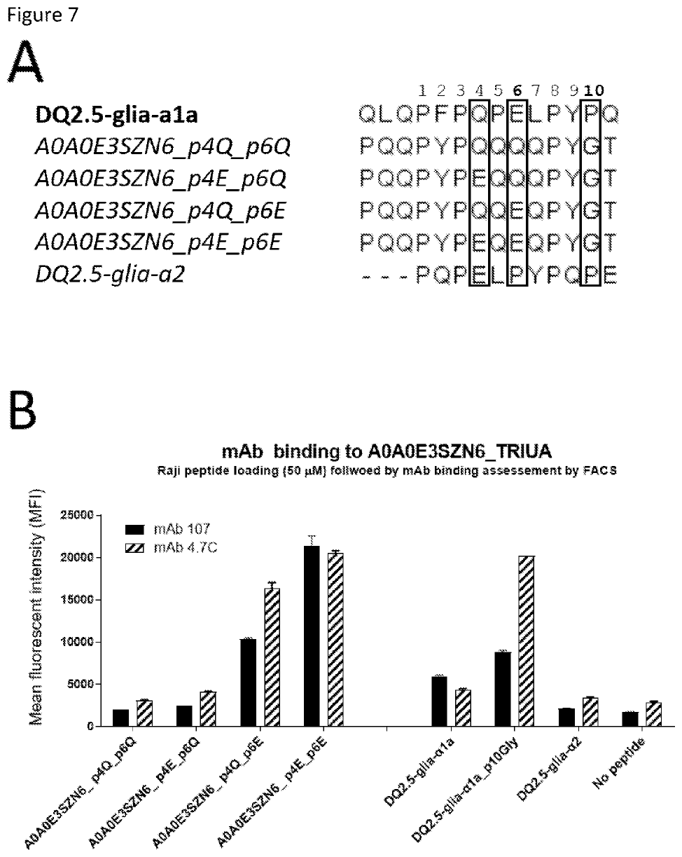

FIG. 7:

Assessment of antibody binding to the A0A0E3SZN6_TRIUA epitope candidate. The A0A0E3SZN6_TRIUA peptide was aligned to the homologous DQ2.5-glia-α1a and DQ2.5-glia-α2 epitopes and Qln (Q) to Glu (E) exchange in the corresponding p4 and p6 positions for comparison. The known P positions are indicated and notably, the DQ2.5-glia-α1a epitope contains the artificial p10 glycine extension (A). The indicated peptides (50 μM) were pulsed onto human DQ2.5*Raji cells and antibody binding was tested in FACS as described (B).

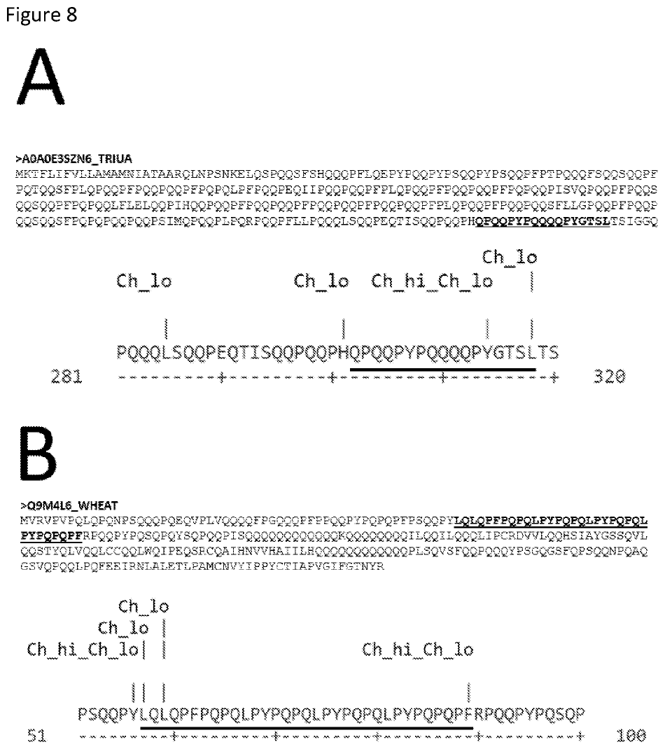

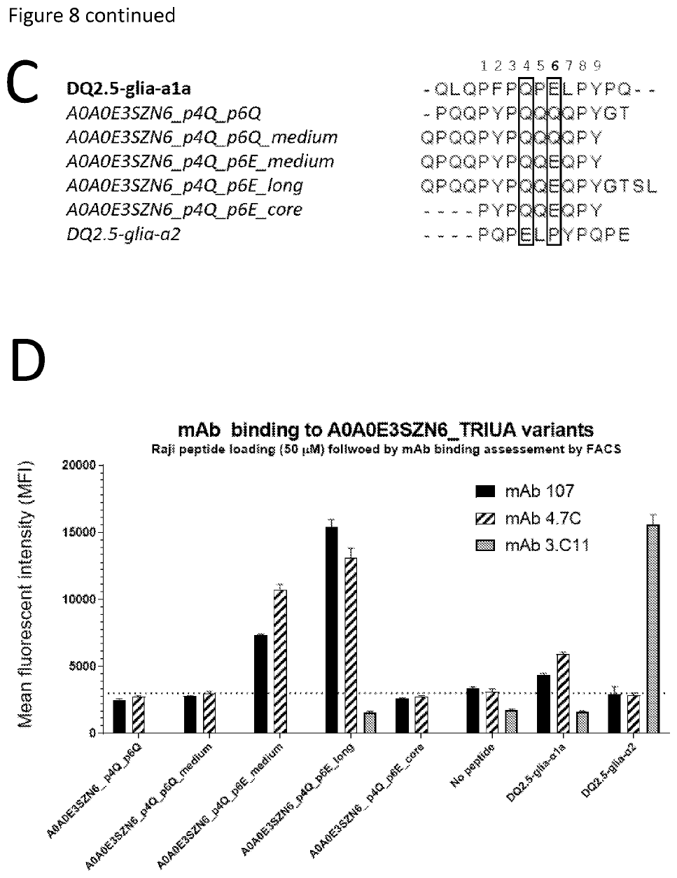

FIG. 8:

Assessment of antibody binding to the physiological relevant forms of the A0A0E3SZN6_TRIUA epitope candidate. The T. urartuA0A0E3SZN6_TRIUA (A) and T. aestivum Q9M4L6_WHEAT (B) sequences were in silico digested with tryspin and chymotrypsin, and the relevant digested fragments shown underlined (A and B). Relevant sequences were aligned to the homologous DQ2.5-glia-α1a and DQ2.5-glia-α2 epitopes and Qln (Q) to Glu (E) exchange in the p6 position for comparison. The known P positions are indicated (C). The indicated peptides were pulsed (50 μM) onto human DQ2.5*Raji cells and antibody binding was tested in FACS as described (D).

FIG. 9:

Assessment of in vitro chymotrypsin proteolysis of the A0A0E3SZN6_TRIUA protein. The T. aestivum Q9M4L6_WHEAT (A), T. urartu A0A0E3SZN6_TRIUA (B) and T. aestivum Q9FUW7_WHEAT (C) proteins where produced and affinity purified from E. coli, followed by in vitro chymotrypsin digest and mass spectrometry analysis of the resulting peptide segments (D, E and F). The anticipated 9-mer core regions of the relevant epitopes are indicated by enumeration (D, E and F). Panel A: SDS-PAGE and Western blot analysis of Q9M4L6_WHEAT. Lane M₁: Protein Marker, GenScript, Cat. No. M00516, Lane M₂: Protein Marker, GenScript, Cat. No. M00521, Lane 1: BSA (2.00 pg), Lane 2: Q9M4L6_WHEAT (Reducing condition, 2.00 pg), Lane 3: Q9M4L6_WHEAT (Non-reducing condition, 2.00 μg), Lane 4: Q9M4L6_WHEAT (Reducing condition), Lane 5: Q9M4L6_WHEAT (Non-reducing condition), Primary antibody: Mouse-anti-His mAb (GenScript, Cat. No. A00186). Panel B: SDS-PAGE and Western blot analysis of A0A0E3SZN6_TRIUA. Lane M₁: Protein Marker, GenScript, Cat. No. M00516, Lane M₂: Protein Marker, GenScript, Cat. No. M00521, Lane 1: BSA (2.00 μg), Lane 2: A0A0E3SZN6_TRIUA (Reducing condition, 2.00 μg), Lane 3: A0A0E3SZN6_TRIUA (Reducing condition), Primary antibody: Mouse-anti-His mAb (GenScript, Cat. No. A00186). Panel C: SDS-PAGE and Western blot analysis of Q9FUW7_WHEAT. Lane M₁: Protein Marker, GenScript, Cat. No. M00516, Lane M₂: Protein Marker, GenScript, Cat. No. M00521, Lane 1: BSA (2.00 μg), Lane 2: Q9FUW7_WHEAT (Reducing condition, 2.00 μg), Lane 3: Q9FUW7_WHEAT (Reducing condition), Primary antibody: Mouse-anti-His mAb (GenScript, Cat. No. A00186).

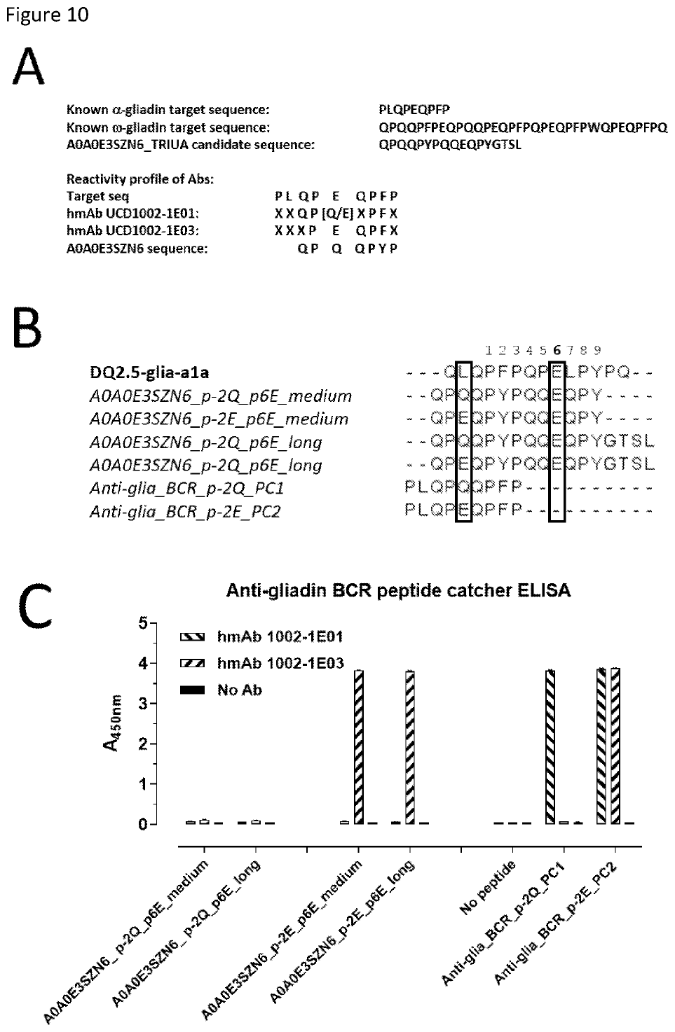

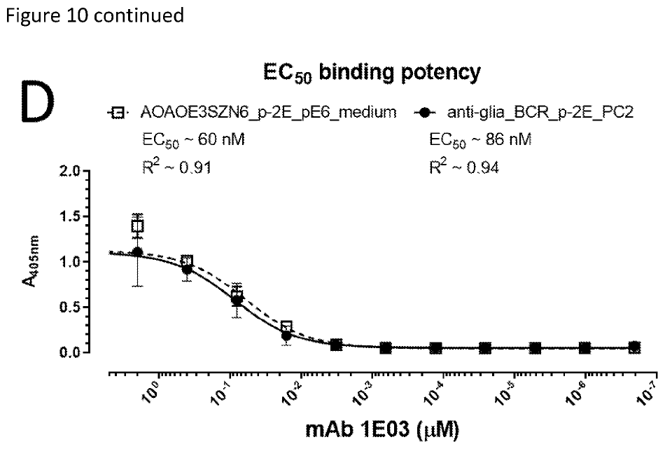

FIG. 10:

Assessment of CeD derived gluten peptide-specific B cell receptor (BCR) binding to the A0A0E3SZN6_TRIUA epitope candidate. Consensus epitope of the CeD prototypic gluten specific BCRs 1 E01/1E03 compared with the documented T. aestivum omega Q9FUW7_WHEAT and T. urartu A0A0E3SZN6_TRIUA epitope candidate sequences (A and B). The 1E01/1E03 BCRs were tested for binding to immobilized peptides in ELISA in the form of reformatted soluble IgG as described (C). EC50 determination for comparison of 1E03 BCR binding potency to the reactive PC2 and A0A0E3SZN6 peptides in the peptide catcher ELISA (D).

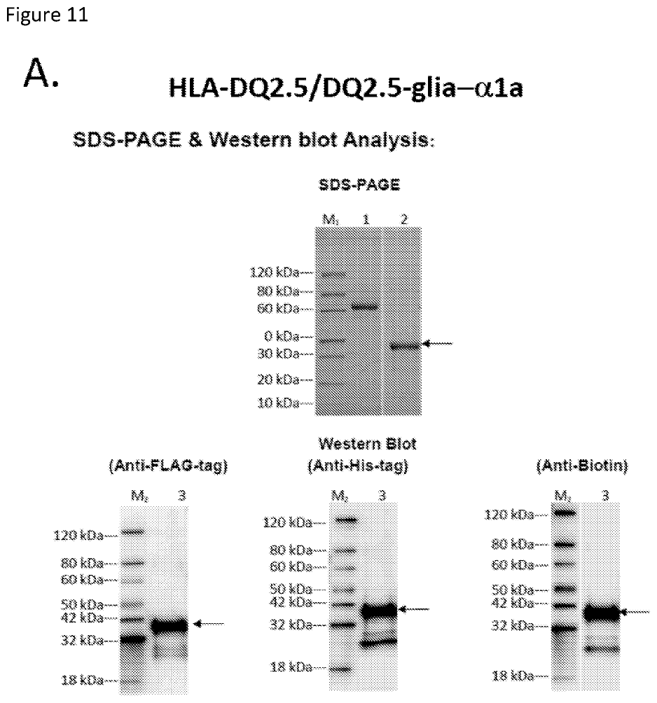

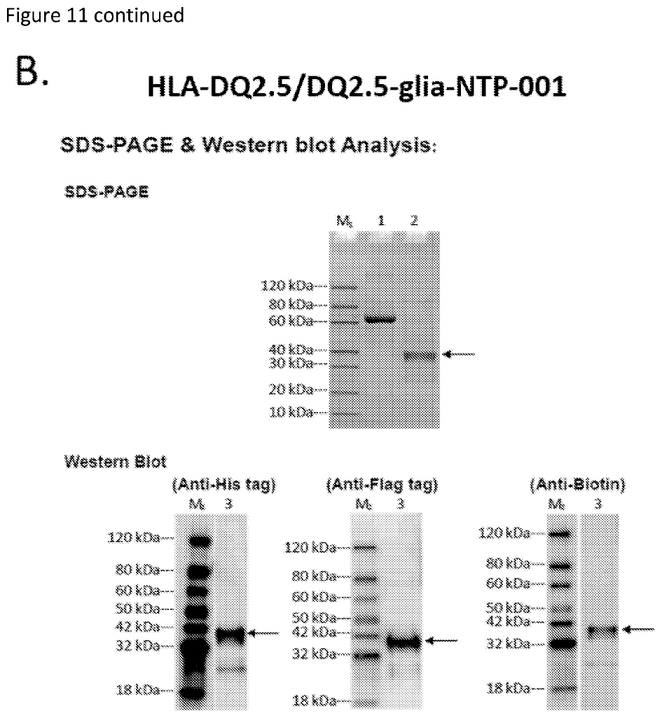

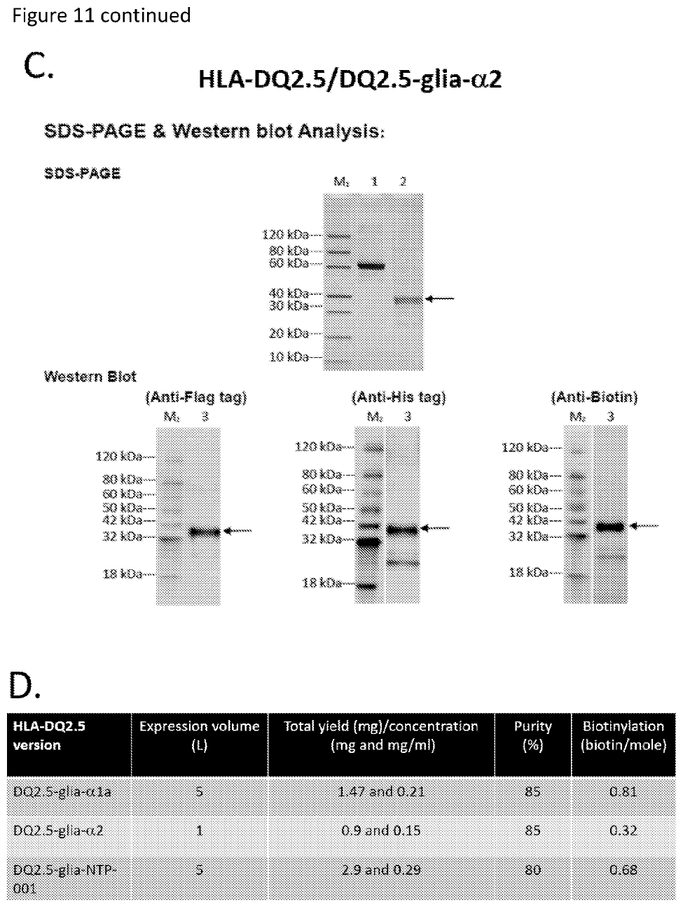

FIG. 11:

Production of recombinant soluble pHLA produced in insect cells. The soluble recombinant versions of HLA-DQ2.5 ectodomains equipped with covalent coupled gliadin peptides using a 15 aa synthetic linker (GAGSLVPRGSGGGGS (SEQ ID NO:56)) were produced by Genscript using Sf9 insect cells and biotinylated essentially as described (Quarsten et al J Immunol, 2001, 167: 4861). Equal amounts of protein were assessed by SDS PAGE and western blot to assess integrity (A to C). The following gliadin peptides were used in the respective versions (A) QLQPFPQPELPY (SEQ ID NO:53), (B) QPEQPYPQQEQPY (SEQ ID NO:31) and (C) PQPELPYPQPE (SEQ ID NO:57), respectively. The deamidated version of the T. urartu A0A0E3SZN6_TRIUA identified sequence comprising both the BCR and T cell epitope candidate sequences (A0A0E3SZN6_p-2E_p6E_medium) is here re-annotated as DQ2.5-glia-NTP-001. Panel A: SDS-PAGE and Western blot analysis of DQB1*0201:DQ25_glia_a1a_DQA1*0501. Lane M₁: Protein Marker, GenScript, Cat. No. M00516, Lane M₂: Protein Marker, GenScript, Cat. No. M00521, Lane 1: BSA (2.00 μg), Lane 2: DQB1*0201:DQ25_glia_a1a_DQA1*0501 (Reducing condition, 2.00 μg), Lane 3: DQB1*0201:DQ25_glia_a1a_DQA1*0501 (Reducing condition), Primary antibody: Mouse-anti-His mAb (GenScript, Cat. No. A00186), Primary antibody: Mouse-anti-FLAG mAb (GenScript, Cat. No. A00187), Primary antibody: Streptavadin-HRP (GenScript, Cat. No. M00091). Panel B: SDS-PAGE and Western blot analysis of DQB1*0201:DQ25_glia_a1a_mutant_DQA1*0501. Lane M₁: Protein Marker, GenScript, Cat. No. M00516, Lane M₂: Protein Marker, GenScript, Cat. No. M00521, Lane 1: BSA (2.00 μg), Lane 2: DQB1*0201:DQ25_glia_a1a_mutant_DQA1*0501 (Reducing condition, 2.00 μg), Lane 3: DQB1*0201:DQ25_glia_a1a_mutant_DQA1*0501 (Reducing condition), Primary antibody: Mouse-anti-His mAb (GenScript, Cat. No. A00186), Primary antibody: Mouse-anti-FLAG mAb (GenScript, Cat. No. A00187), Primary antibody: Streptavadin-HRP (GenScript, Cat. No. M00091). Panel C: SDS-PAGE and Western blot analysis of DQB1*0201:DQ25_glia_a2_DQA1*0501. Lane M₁: Protein Marker, GenScript, Cat. No. M00516, Lane M₂: Protein Marker, GenScript, Cat. No. M00521, Lane 1: BSA (2.00 μg), Lane 2: DQB1*0201:DQ25_glia_a2_DQA1*0501 (Reducing condition, 2.00 μg), Lane 3: DQB1*0201:DQ25_glia_a2_DQA1*0501 (Reducing condition), Primary antibody: Mouse-anti-His mAb (GenScript, Cat. No. A00186), Primary antibody: Mouse-anti-FLAG mAb (GenScript, Cat. No. A00187), Primary antibody: Streptavadin-HRP (GenScript, Cat. No. M00091).

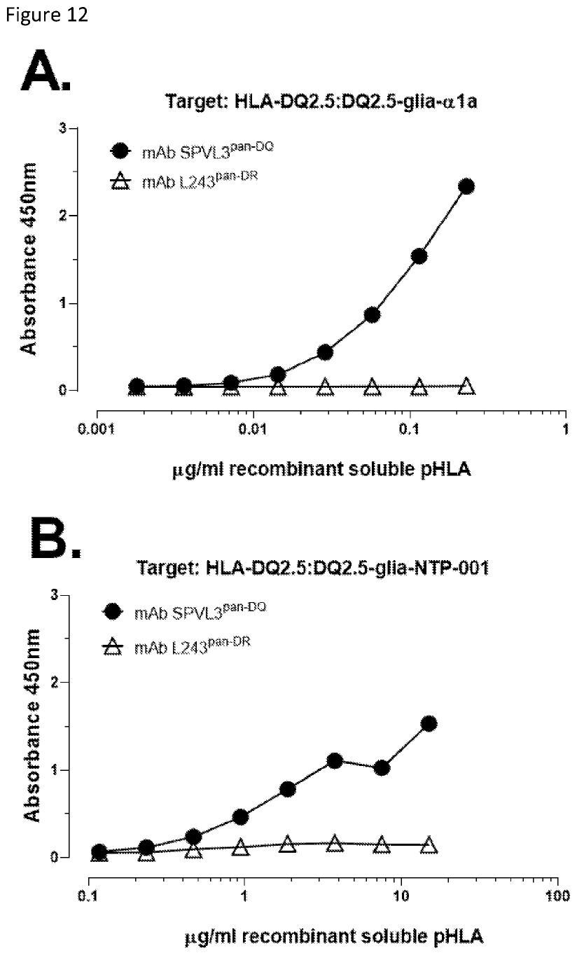

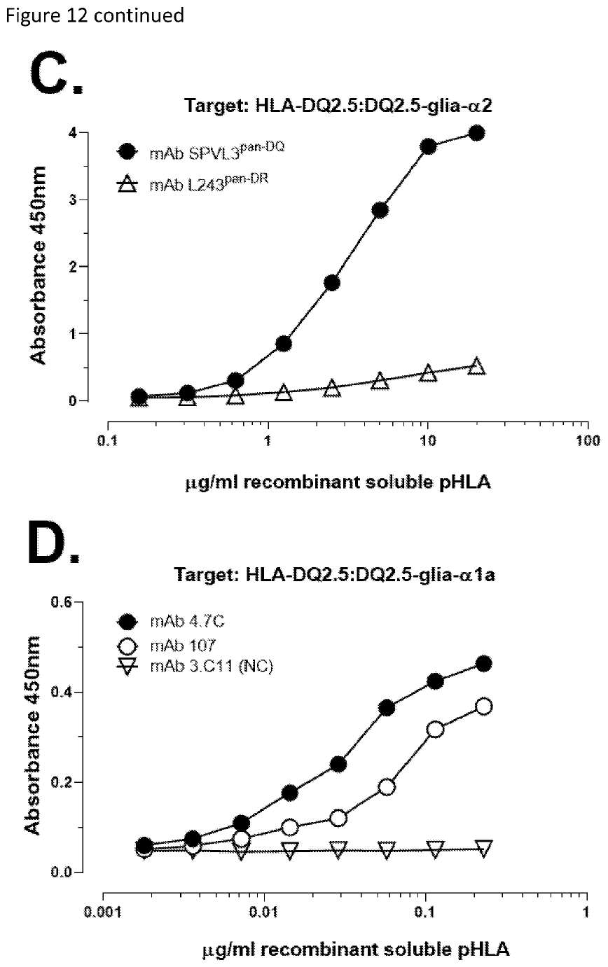

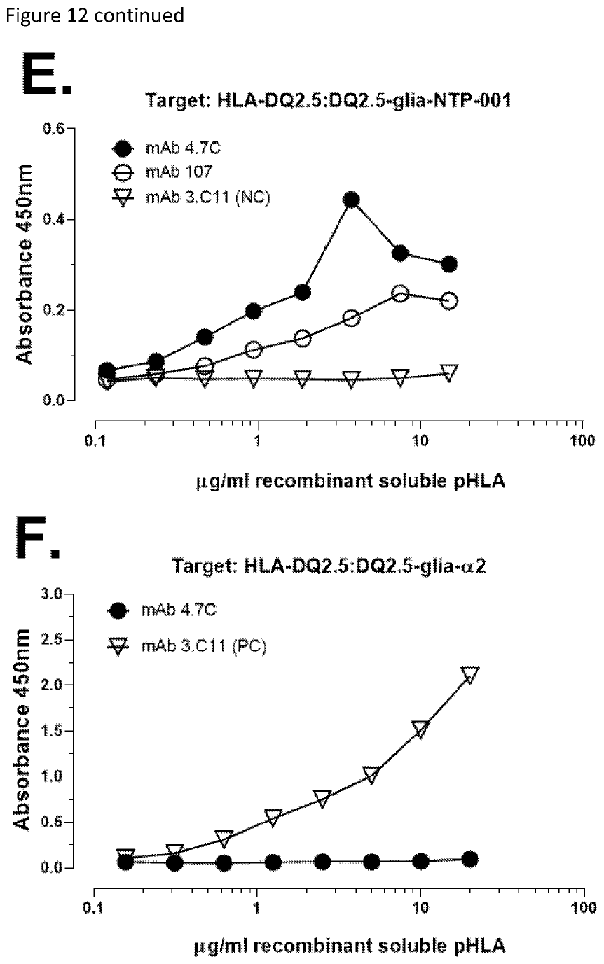

FIG. 12:

Assessment of antibody binding to recombinant soluble pHLA (rs-pHLA) produced in insect cells. Biotinylated soluble recombinant versions of HLA-DQ2.5 equipped with the indicated peptides were immobilized on neuravidin-coated wells and assessed for reactivity to the indicated antibodies (mAb) in ELISA. (A to C) Detection of rs-pHLA by use of conformation specific pan-HLA-DQ (mAb SPVL3) and pan-HLA-DR (mAb L243). (D to F) Detection of individual rs-pHLAs by use of the TCR-like mAbs 107, 4.7C and 3.C11, respectively.

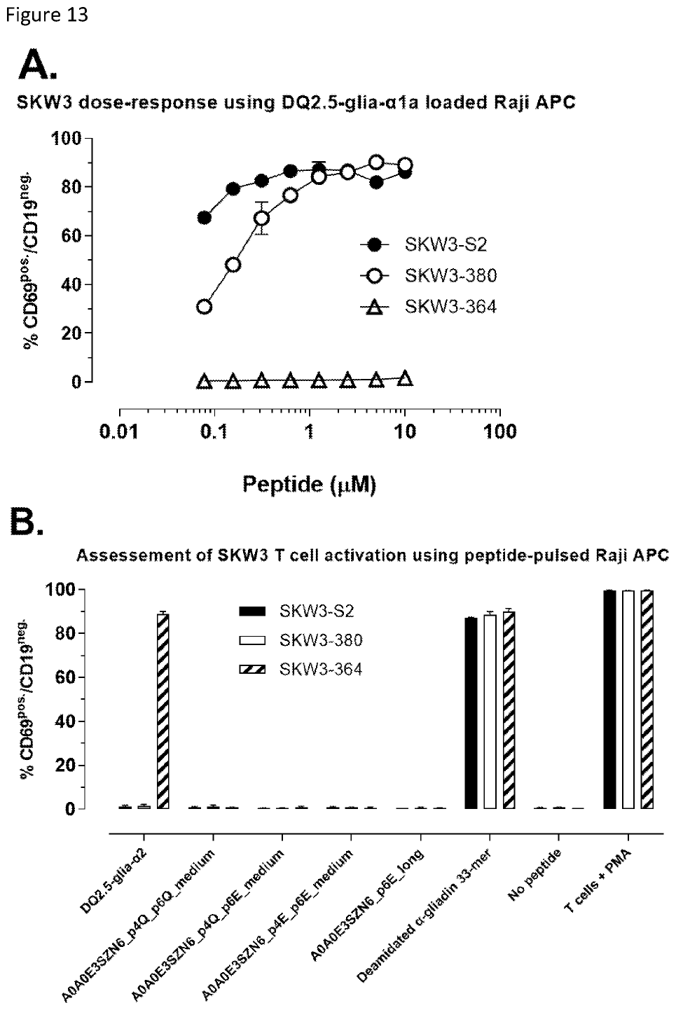

FIG. 13:

Assessment of T cell activation using CeD patient derived TCR-reconstructed SKW3 T cells. (A) Raji B cells were loaded with a serial dilution of DQ2.5-glia-α1a peptide (N-QLQPFPQPELPY-C(SEQ ID NO:53)) and co-cultured with engineered gliadin-specific SKW3 T cells. T-cell activation was measured as CD69+CD19− cells in flow cytometry. (B) Raji B cells were loaded with a 10 μM of the indicated peptide and co-cultured with engineered gliadin-specific SKW3 T cells. T-cell activation was measured as CD69+CD19-cells in flow cytometry. The following peptides were employed: DQ2.5-glia-α2 (N-PQPELPYPQPE-C(SEQ ID NO:57)), A0A0E3SZN6_p4Q_p6Q_medium (N-QPQQPYPQQQQPY-C(SEQ ID NO:20)), A0A0E3SZN6_p4Q_p6E_medium (N-QPQQPYPQQEQPY-C(SEQ ID NO:28)), A0A0E3SZN6_p4E_p6E_medium (N-QPQQPYPEQEQPY-C(SEQ ID NO:58)), A0A0E3SZN6_p6E_long (N-QPQQPYPQQEQPYGTSL-C(SEQ ID NO:30)), respectively. Phorbol myristate acetate (PMA) was used a positive control to assess peptide-independent maximum T cell activation, and baseline threshold on non-activated T cells were set by co-culturing SKW3 and Raji cells in the absence of peptide, respectively.

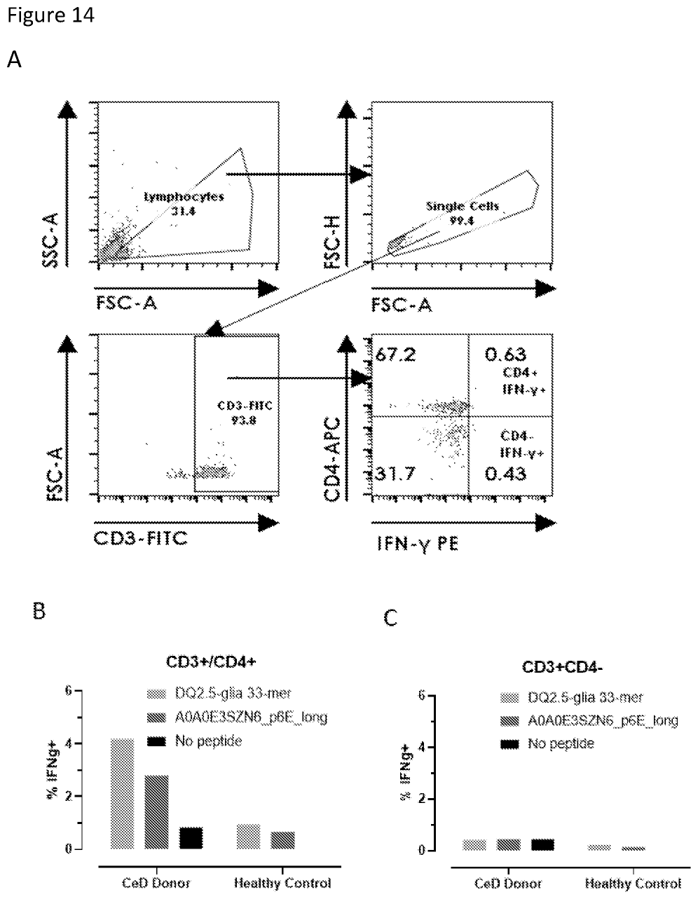

FIG. 14:

Intracellular IFNγ flow assessment of CD4 T cell peptide stimulatory capacity using PBMCs. Cryopreserved HLA-DQ2.5 typed PBMCs from confirmed healthy controls (HC) and CeD patients were purchased (HemaCare-Cellero) and used in autologous in vitro T cell peptide stimulation assays followed by intracellular IFNγ staining in flow. (A) PBMCs from CeD donor 595 were used to set intracellular IFNγ baseline detection gates on CD3/CD4 T cells using anti-IFNγ-PE. (B and C) Bar graph representing intracellular IFNγ detection in CD4+/IFNγ+(B) and CD4−/IFNγ+(C) gates as illustrated in A. PBMCs from CeD donor 595 and HC 575 were stimulated with 20 μM DQ2.5-glia 33-mer (N-LQLQPFPQPQLPYPQPQLPYPQPQLPYPQPQPF-C(SEQ ID NO:59)) and A0A0E3SZN6_p6E_long (N-QPQQPYPQQEQPYGTSL-C(SEQ ID NO:30)), for 48h followed by intracellular T cell IFNγ detection.

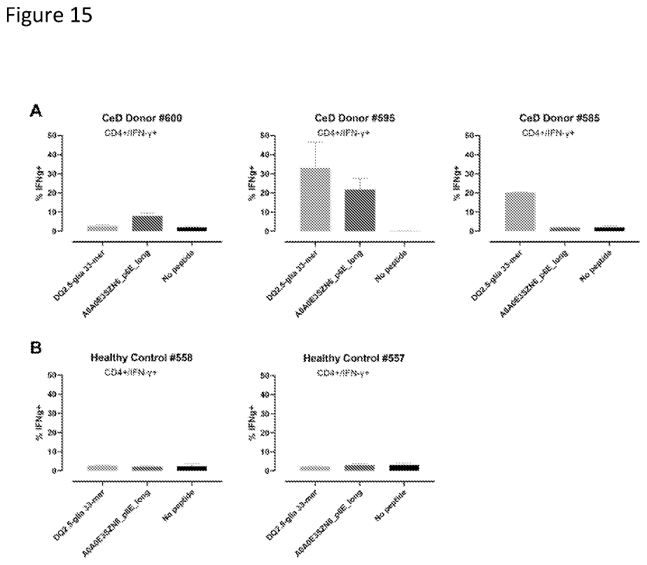

FIG. 15:

Intracellular IFN-γ flow assessment of CD4 T cell peptide stimulatory capacity using PBMCs. Cryopreserved HLA-DQ2.5 typed PBMCs from confirmed healthy controls (HC) and CeD patients were purchased (HemaCare-Cellero) and used in autologous in vitro T cell peptide stimulation assays followed by intracellular IFN-γ staining in flow. Intracellular IFN-γ baseline detection gates on CD3/CD4 T cells using anti-IFN-γ-PE were set as in FIG. 14.

PBMCs from CeD donors (585, 595 and 600) and HCs (557 and 558) were stimulated with 20 μM DQ2.5-glia 33-mer (N-LQLQPFPQPQLPYPQPQLPYPQPQLPYPQPQPF-C(SEQ ID NO:59)) and A0A0E3SZN6_p6E_long (N-QPQQPYPQQEQPYGTSL-C(SEQ ID NO:30)), for 48h followed by intracellular T cell IFN-γ detection (A and B). Values are presented as mean values and ±SEM.

### EXAMPLES

### Example 1. Identification of a New T Cell Epitope Associated with Celiac Disease

### Materials and Methods

### Human PBMCs, Peptides and Selected Antibodies

Frozen HLA typed purified human PBMCs from confirmed celiac disease (CeD) patients and healthy controls (HCs) were purchased from HemaCare-Cellero (https://cellero.com/).

All peptides were purchased at ≥85% purity from Genscript (http://www.genscript.com). The pan anti-DQ (SPV-L3) and pan anti-DR (L243) antibodies were purchased from Beckman-Coulter and Thermo Scientific, respectively.

Recombinant pHLA Expression, Purification, and Validation

Recombinant HLA-DQ2.5 molecules with covalently coupled gluten-derived peptides containing the T-cell epitopes DQ2.5-glia-α1a (QLQPFPQPELPY (SEQ ID NO:53), underlined 9mer core sequence), DQ2.5-glia-α2 (PQPELPYPQPE (SEQ ID NO:57)), DQ2.5-glia-NTP-001 (QPEQPYPQQEQPY (SEQ ID NO:31)) were generated by Genscript (http://Iwwv.enscrit.com) essentially as previously described (Quarsten, H., et al., 2001, supra), with the additon of C-terminal FLAG and HIS tags on the α and β-chains to facilitate affinity purification and recombinant protein detection. Briefly, Sf9 insect cell produced soluble, in vivo biotinylated recombinant pMHC was affinity purified using anti-FLAG, concentrated and the proteins were analyzed by SDS-PAGE and Western blot by using standard protocols for molecular weight and purity measurements.

### Recombinant Antibody Expression, Purification, and Validation

Recombinant human IgG1 proteins were produced by Genscript (http://www.genscript.com). In brief, the respective antibody variable (V) genes were manufactured by gene synthesis and cloned in-frame into human constant heavy (H) and light (L) chain genes into the eukaryotic expression vector pcDNA3.4. Proteins were produced in Expi293F cells and affinity purified on protein A and SEC, followed by SDS-PAGE and Western blot by using standard protocols for molecular weight and purity measurements. The V genes for clone 107 and 4.7C are shown in Table 1 and Table 2, respectively, and for clones 1002-1E01 and 1002-1E03 from PDB IDs 5IHZ and 51K3, respectively (Snir et al., 2017, JCI Insight, 2(16):e93961).

### Epitope Candidate Pattern Search and Identification

To identify putative gliadin-derived sequences different from, but with similarity to, the 30 DOQ.2.5-glia-α1a epitope (PFPQP\vec{E}LPY (SEQ ID NO:4)), the UniProtKB protein database was searched using the ScanProsite tool (https://prosite.expasyorg/scanprosite/). Different motifs were used but preserved key residues of the DQ.2.5-glia-α1a epitope, and some searches also allowed length variation in the N-terminal and C-terminal sequence beyond the 9-mer core. More specifically, we fixed the proline (P) in p1, the glutamine (Q) in p6, the tyrosine (Y) in p9, and P was disallowed in the position p10 after the p9 Y (underlined). To focus the output of the searches, we restricted the output to the Triticum taxonomic group. 14-mer peptides of putative hits were synthesized and tested for binding to the 107 and 4.7C antibodies when loaded onto Raji cells as described. In some cases, Q residues were exchanged with glutamate (E) in the peptide synthesis in order to mimic positional deamidation.

### Recombinant Gliadin Expression, Purification, and Validation

Recombinant wheat gliadin proteins were produced by Genscript (http://www.genscript.com) essentially as described (Arentz-Hansen E H. et al., Gut 2000; 46:46-51). In brief, the respective gliadin genes (Uniprot codes Q9M4L6, Q9FUW7 and A0A0E3SZN6) were manufactured by gene synthesis appending C-terminal HIS tags, and cloned in-frame into the bacterial expression vector pET17b. Proteins were produced in E. coli BL21 (DE3)pLysS cells and purified from whole cell lysate under denatured conditions using two-step purification by ethanol precipitation and salt precipitation, followed by SDS-PAGE and Western blot by using standard protocols for molecular weight and purity measurements.

### ELISA

The peptide capture ELISAs were performed as follows. Briefly, 96-well MaxiSorp microtiter plates (Nunc) were coated overnight at 4° C. with NeutrAvidin (Avidity, 5 μg/ml in PBS), before blocking with 2% biotin-free skim milk powder in PBS (w/v). The various peptides (all synthesized with a biotinylated N-terminal GSGSGS extension) were diluted to 10 μg/ml in PBSTM (PBS with 2% biotin-free skim milk powder (w/v) and 0.05% Tween-20 (v/v)) and captured onto the NeutrAvidin. The 1E01/1E03 antibodies were diluted in PBSTM to 5 μg/ml, added to the wells and detected with either polyclonal rabbit anti-human (Sigma, 1:10000) in PBSTM, respectively, and developed by addition of TMB solution (Calbiochem) before absorbance reading at 620 nm (450 nm in the case of HCl addition), respectively. Assays were performed at RT with duplicate wells. Between each layer, the plates were washed 3-5× with PBST.

The pHLA-specific ELISAs were performed as follows. Briefly, 96-well MaxiSorp microtiter plates (Nunc) were coated overnight at 4° C. with NeutrAvidin (Avidity, 5 μg/ml in PBS), before blocking with 2% biotin-free skim milk powder in PBS (w/v). The various biotinylated pHLAs were diluted to 20 μg/ml in PBSTM and captured onto the NeutrAvidin. The various antibodies were diluted in PBSTM to 5 μg/ml, added to the wells and detected with either polyclonal rabbit anti-human (Sigma, 1:10000) or anti-mouse IgG-HRP (Sigma, 1:2000) in PBST, respectively, and developed by addition of TMB solution (Calbiochem) before absorbance reading at 620 nm (450 nm in the case of HCl addition), respectively. Assays were performed at RT with duplicate wells. Between each layer, the plates were washed 3-5× with PBST.

### Gliadin Proteolytic Digestion and Mass Spectrometry (MS)

Recombinant gliadins were in vitro chymotrypsin digested essentially as described (Molberg O, et al., Methods Mol Med. 2000; 41:105-24). Briefly, about 1 mg recombinant gliadin was digested with chymotrypsin (Sigma) at 200:1 (w:w) in 0.1M NH₄HCO₃ with 2M urea at 37° C. for 24h followed by enzyme inactivation for 5 min at 95° C. and channeled further into MS analysis essentially as described (Dorum S., et al., 2016, Sci Rep. 6:25565). The MS analysis was performed by the University of Oslo (UiO) proteomics core facility at the Department of Biosciences. MS spectra were analyzed by using the PEAKS studio software (Bioinformatics Solutions Inc.) searched against a custom databased made by the respective gliadin Uniprot accession codes.

### Retroviral Transduction of Human SKW3 T Cells and Flow Cytometry

The T cell receptor (TCR) reconstructed SKW3 clones SKW3-380 and SKW3-364 have been described (Frick R., et al., 2021, Sci. Immunol. 6(62):eabg4925). The SKW3-S2 cells were generated essentially in the same manner using the TCR V gene sequences from PFB ID 40ZI (Petersen et al., Nat Struct Mol Biol 2014, 21(5), 480-488). In brief, TCR V gene sequences were reconstructed by gene synthesis as human/mouse chimeric TCRs and cloned into pMSCV (Clontech Laboratories) by Genscript (http://www.censcript.com). Retroviral transduction of the SKW3 human T cells (CLS Cell Lines Service GmbH) was performed using the Retro-X Universal Packaging System (Clontech) according to the manufacturer's instructions. Stable, homogenous TCR-expressing SKW3 T cells were obtained by standard cell expansion and FACS sorting using a FACSAria II cytometer (BD Biosciences) based on their TCR expression levels assessed by H57-Alexa647 (Thermo Fisher Scientific) antibody staining. The TCR transduced SKW3 cells were validated for peptide-specific activation using Raji cells as antigen presenting cells, essentially as described (Frick, R., et al., 2021, supra). T-cell activation was measured by CD69 up-regulation assessed by anti-human CD69-APC (BD Biosciences) antibody staining. Data was acquired on a BD Accuri C6 cytometer (BD Biosciences) and analyzed using FlowJo software V10 (Tree Star).

### T Cell Activation and Inhibition Assays

For T-cell activation assays 50,000 Raji B cells were incubated in RPMI/10% FCS at 37° C./ON with titrated amounts of DQ2.5-glia-α1a (QLQPFPQPELPY (SEQ ID NO:53)) peptide, followed by washing to remove remaining free peptide and addition of 40,000 SKW3 T cells. Cells were cultured at 37° C./ON before they were analyzed in flow cytometry. As a control, Cell Stimulation Cocktail containing PMA and ionomycin (eBioscience, 1:500) was added to wells containing SKW3 T cells only. Based on the established dose-response in T-cell activation, a peptide concentration estimated to result in about 60% T-cell activation (measured as CD69 upregulation on the CD19neg population) was chosen for the inhibitory assay. Following ON incubation with peptide as above and washing, 1 μM (final concentration) of either 4.7C or 3.C11 were added to the Raji cells, before T cells were added and incubation continued ON. The resulting T-cell activation was measured as above. As control Abs, either 0.1 μM (final concentration) of pan-anti-DR or pan-anti-DQ were added in parallel.

### Intracellular IFN-γ Flow Cytometric Detection of Peptide-Activated PBMCs

Peptide-stimulated PBMCs were assessed for intracellular IFN-γ following reagents and the standard protocol from BioLegend (https://www.biolegend.com/). Briefly, cryopreserved human HLA-DQ2.5⁺ PBMCs (HemaCare-Cellero) where gently thawed and washed in ice-cold PBS before resuspended into RPMI1640 supplemented with 10% FCS (v/v) before volumes of 1 ml (about 2×10⁷ cells) were aliquoted into a 24-well microtiter plate (NUNC). One set of wells received either 20 μM of peptide (either A0A0E3SZN6_p6E_long (QPQQPYPQQEQPYGTSL (SEQ ID NO:30)) or 33-mer (LQLQPFPQPELPYPQPELPYPQPELPYPQPQPF (SEQ ID NO:55))). The remaining cells did not receive any peptide. The cells were grown at standard conditions at 37° C. for 36h before Brefaldin A (BioLegend) and Monensin (BioLegend) was added, and incubation continued for another 12 h. The cells were then assessed for intracellular IFN-γ by flow. Data was acquired on a BD Accuri C6 cytometer (BD Biosciences) and analyzed using FlowJo software V10 (Tree Star).

### Results

We have previously identified a peptide-MHC (p-MHC) antibody that can bind to HLA-DQ2.5:DQ2.5-glia-α1a, i.e. the antibody can bind to the MHC class II molecule HLA-DQ2.5 when associated with the coeliac disease (CeD) associated glia-α1a epitope (PFPQPQLPY (SEQ ID NO:60)), i.e. the antibody can bind to a pMHC complex.

This antibody (referred to as the 107 antibody) has been shown to specifically react with samples taken from CeD patients and in an HLA specific manner, i.e. the antibody is CeD specific and also specific for the HLA molecule HLA-DQ2.5 (see FIG. 1).

An affinity matured version of this antibody was also generated (referred to as the 4.7C antibody), which has a higher affinity for binding to the pMHC complex than the parent antibody, 107 (see FIG. 2).

Detection of Cell-Surface pMHC

The above experiments were carried out by assessing binding to soluble recombinant pMHC molecules. However, the 4.7C antibody also showed good and peptide-specific ability to bind antigen presenting cells (A20 mouse B cells) which had been engineered to recombinantly express pMHC complexes on the surface in the form of HLA-DQ2.5 with covalently linked αla peptide, as well as control peptides (FIG. 3A). A 12-mer peptide was used (QLQPFPQPELPY (SEQ ID NO:53)) containing the minimum αla peptide (underlined). In this experiment, the CeD associated form of the epitope was used, i.e. containing an E rather than a Q residue (PFPQPELPY (SEQ ID NO:4) as opposed to PFPQPQLPY (SEQ ID NO:60)).

Interestingly, when the antibody 4.7C was tested on human antigen presenting cells with native MHC expression at physiological levels (Raji cells) that had been externally loaded with the same 12-mer containing the minimal epitope, but now as soluble peptide (i.e. peptide pulsed cells), the binding was markedly weaker (FIG. 3B). In addition, the antibody showed no binding to these antigen presenting cells when externally loaded with a gluten 33-mer peptide (FIG. 3B). The α-gliadin 33-mer (LQLQPFPQPELPYPQPELPYPQPELPYPQPQPF (SEQ ID NO:55)) which is believed to be a naturally occurring fragment, and is strongly associated with CeD, contains one copy of the minimal α1a epitope and three overlapping copies of the DQ2.5-glia-α2 epitope These results were surprising given the binding results for the recombinantly expressed αla peptide. However, there are a number of possible reasons for this, including the possibility that the interaction of the 12-mer or 33-mer with the HLA-DQ2.5 is not stable enough after loading with exogenous peptide, or the fact that there are likely to be far fewer pMHC complexes on the cell surface (lower density pMHC expression) as the native Raji cells do not express as much MHC as the cells in which the pMHC is recombinantly overexpressed. Despite these possible confounding parameters, the 33-mer is known to bind better to the HLA molecule than shorter peptides, which also is seen in e.g. T cell activation assays (Gunnarsen et al., 2017, JCI Insight; 2(17):e95193), hence in comparison with the 12-mer, it was expected that the 33-mer would show better staining than the 12-mer, which it did not.

### Inhibition of T Cell Activation

The antibody 4.7C was also tested for the ability to inhibit T-cell activation in vitro using a human T cell line (SKW380) which expresses TCRs specific for DQ2.5-ala. Raji cells loaded with titrated amounts of stimulatory gliadin peptide (QLQPFPQPELPY (SEQ ID NO:53)) were co-cultured with the SKW380 T cells and T cell activation was measured by determining CD69 expression on the SKW380 cells using flow cytometry (FIG. 4A). A peptide concentration inducing 60% T-cell activation was chosen for the subsequent experiment where the peptide loaded Raji cells were incubated with antibodies upon addition of SKW380 T cells in order to assess their T-cell inhibitory capacity (FIG. 4B).

Although around 20% inhibition of T cell activation was observed and this inhibition was specific, the level of inhibition might be expected to be higher.

### Staining Human Small Intestinal Biopsy Material

When tested on CD19+CD45+ plasma cells derived from small intestinal biopsy samples taken from the inflamed mucosa of untreated, confirmed HLA-DQ2.5+, CeD patients and control subjects, the high affinity (4.7C) and parent (107) antibody showed good staining of the CeD material, showing that the antibodies are detecting HLA-DQ2.5 associated gluten peptide presentation on cells derived from CeD patients (FIG. 5, each patient is shown as a separate circle).

These data were fully in line with a previously reported extensive characterisation of the 107 antibody and its ability to fully disease, HLA and epitope specifically detect peptide presentation in similar CeD patient material (Hoydahl et al., 2019, Gastroenterology 156(5), 1428-1439).

Interestingly, despite the 4.7C antibody having been selected for higher affinity binding to the DQ2.5-glia-α1a epitope (FIG. 2A), in the patient samples the level of staining was fairly similar for the two antibodies (FIG. 5A).

### Investigation of Possible New T Cell Epitope Sequence

To gain better insight into this apparent discrepancy in binding behaviour and lower than expected ability to inhibit T-cell activation, it was contemplated whether it was possible that the antibodies 4.7C and 107 could also recognise an additional peptide sequence/T cell epitope in CeD patients.

For example, when the experiments that had been done were considered more closely it was noted that the 12-mer used for peptide pulsing experiments had the 9-mer minimal T-cell epitope at the C-terminal end, i.e. (QLQPFPQPELPY (SEQ ID NO:53)), whereas the 33-mer sequence is longer with additional sequences at the C-terminal end (LQLQPFPQPELPYPQPELPYPQPELPYPQPQPF (SEQ ID NO:55)). Itwas hypothesised that it was possible that the residue at position 10 onwards (i.e. the positions after the 9-mer minimal T cell epitope PFPQPELPY (SEQ ID NO:4)) might be affecting the ability of the antibodies to bind to the peptides and that the antibodies might also be able to recognise similar T cell epitope sequences associated with CeD but where there was an alternative residue (not P) at the C-terminal end.

For example, other binding studies showed that a Gly (G) residue at position 10 resulted in a good level of antibody binding with the 4.7C and 107 antibodies as compared to having a naturally occurring Pro at p10 extending C-terminally of the minimal DQ2.5-glia-α1a epitope (data not shown).

In summary, the data describing the binding properties of the two antibodies 107 and 4.7C, respectively, clearly showed that both had the capacity to bind specifically to the DQ2.5-glia-α1a peptide epitope as soluble recombinant pHLA as well as pHLA on cells, including cells from CeD patient material. However, the data also showed that both antibodies lacked the capacity to bind the epitope when present on peptide versions having the naturally occurring proline residue occupying the p10 position, which also include the anticipated important 33-mer peptide from wheat α-gliadin (Dorum et al., J Immunol Nov. 1, 2014, 193 (9) 4497-4506). There is an acknowledged gap in epitope insight in CeD, where only up to 50% of the patient reactivity can be accounted for at epitope resolution (Raki et al., 2017, Gastroenterology 153(3) 787-798). In light of the specific and clearly defined binding profile of the 107 and 4.7C antibodies, it therefore became a possibility that the somewhat unexpected similar patient material staining levels were caused by their additional binding to yet undiscovered gluten epitopes.

To try and investigate this, database searching was carried out using the original 9-merT cell epitope sequence PFPQPQLPY (SEQ ID NO:60) (and the deamidated version PFPQPELPY (SEQ ID NO:4)), together with the 12-mer sequence used for the Raji cell peptide pulsing experiments QLQPFPQPELPY (SEQ ID NO:53) (and the native/healthy version QLQPFPQPQLPY (SEQ ID NO:61)) and various amino acid changes to these sequences, based on known shared importance of certain positions in the epitope, such as the apparent invariant requirement for leucine in p7 on T cell reactivity (Petersen et al., Nat Struct Mol Biol 2014, 21(5), 480-488; Dahal-Koirala et al., Mucosal Immunol 9, 587-596, 2016). In particular a focus was made to find a naturally existing gliadin peptide (i.e. a wheat originating peptide) that was similar to the α1a 9-mer/12-mer but which had an alternative residue at the extension into position 10 (i.e. not proline) and which therefore could potentially result in good binding by the antibodies 107 and 4.7C.

In carrying out this work many candidate sequences were identified and eliminated. For example, it was observed that candidate sequences with Tyrosine, Serine and Alanine residues at position 10 did not show any significant binding to the 4.7C antibody. These observations were corroborated with the already known binding profiles outlined.

Eventually several further candidate sequences were identified through a more liberal ScanProsite (https://prosite.expasy.org/scanprosite/) patter search of the UniProt Knowledge Base (https://www.uniprot.org/), which had less (but still reasonable) similarity with the 9-mer, including having a Y residue at position 9, which had been identified as likely to be important for the conformation of the epitope being recognised, but also having a G residue at position 10. More of these candidates were tested for binding to the 4.7C antibody and one of these sequences showed particularly promising results.

This candidate peptide (FIG. 6A) is found as part of an omega-gliadin protein in a wild form of wheat termed red wild einkorn (Triticum uratu, UniProtKB ID: A0A0E3SZN6_TRIUA, SEQ ID NO:1, FIG. 6B).

This peptide showed some similarity to the α1a 9-mer/12-mer but also has a G residue at position 10 in relation to the 9-mer sequence. This sequence had a core 9-mer of PYPQQQQPY (SEQ ID NO:8) (FIG. 6A).

This sequence looked like a potential candidate for a CeD T cell epitope as it had some similarity with the α1a epitope (although in fact is classified as an omega-gliadin) and also occurred naturally in a form of wheat. In addition, glutamine (Q) residues were present which would be potential targets for deamidation modification into E residues in CeD patients by the TG2 enzyme (which targets QXP motifs, Sollid et al., 2002, Nat Rev Immunol 2, 647-655), see position 6 of the 9-mer (underlined above) and FIG. 6A. However, in other respects this sequence does not correspond to classic or known CeD T cell epitopes. Indeed, the use of a recognized T cell epitope predictor (NetMHCIIpan 4.0) on the T. urartu sequence did not predict the sequence to be a HLA-DQ2.5 binding peptide.

Four different forms of this peptide (see FIG. 7A) were tested by loading them onto Raji cells as described above and assessing the binding of both the 107 and 4.7C antibodies by FACS (FIG. 7B). The peptides which were assessed were i) PQQPYPQQQQPYGT (SEQ ID NO:24) (referred to as A0A0E3SZN6_p4Q_p6Q, or “QQ”, which corresponds to a native/healthy form of the peptide that contains Q residues at positions 4 and 6 of the candidate T cell epitope 9-mer); ii) PQQPYPEQQQPYGT (SEQ ID NO:62) (referred to as A0A0E3SZN6_p4E_p6Q, or “EQ”, which corresponds to a form of the peptide that contains an E rather than a Q residue at position 4 of the candidate T cell epitope 9-mer); iii) PQQPYPQQEQPYGT (SEQ ID NO:29) (referred to as A0A0E3SZN6_p4Q_p6E, or “QE”, which corresponds to a form of the peptide that contains an E rather than a Q residue at position 6 of the candidate T cell epitope 9-mer); iv) PQQPYPEQEQPYGT (SEQ ID NO:63) (referred to as A0A0E3SZN6_p4E_p6E, or “EE”, which corresponds to a form of the peptide that contains E rather than Q residues at positions 4 and 6 of the candidate T cell epitope 9-mer). Both antibodies were shown to bind well to the QE and EE peptides but not to the QQ and EQ forms (FIG. 7B). Peptides containing an E at position 6 of the T cell 9-mer epitope are more likely to be found in patients with CeD as this residue is part of a classic TG2 target sequence, QXP (here QQP). Notably, the classical DQ2.5-glia-α1a epitope, as well as an artificial version containing a p10 glycine extension were included as controls in this assay. As expected, the antibodies bound to the artificial version, whereas low level binding was seen to the classical form. No binding was observed to the homologous DQ2.5-glia-α2 epitope.

It was also assessed how the antibodies reacted with more physiologically realistic forms of this peptide, which would be versions likely to exist in CeD patients, by carrying out an in-silico trypsin/chymotrypsin digest of the T. uratu sequence (FIG. 8A). As control, the T. aestivum α-gliadin sequence encoding the 33-mer peptide was included in parallel (FIG. 8B). This identified two possible physiological forms of the sequence, QPQQPYPQQQQPY (SEQ ID NO:20) (sometimes referred to herein as “medium” peptides) and QPQQPYPQQQQPYGTSL (SEQ ID NO:22) (sometimes referred to herein as “long” peptides). These two peptides were tested in the Raji peptide loading assays as described above (p4Q_p6Q medium, p4Q_p6E medium and p4Q_p6E long (FIG. 8C), with Gln (Q) to Glu (E) substitutions in line with that of the likely TG2 activity (QXP) found in CeD patients.

Indeed, both the 107 and 4.7C antibodies were shown to bind well to the E versions of these anticipated physiologically relevant peptides, and the length was crucial for this binding, as the p6 deamidated 9-mer showed no binding (FIG. 8D). Importantly, the isotype-matched DQ2.5-glia-α2 specific 3.C11 antibody did not exhibit any binding to any of the T. urartu sequences, evidencing the specific binding of 107 and 4.7C (FIG. 8D). It is interesting to note here that the results with both the p6 deamidated forms showed a level of binding for the 107 and 4.7C antibodies, which reflects the levels observed when these antibodies were tested on material (antigen presenting cells in the form of CD19+ plasma cells) from CeD patients (FIGS. 1 and 5). Thus, it is believed that a new T cell epitope with the defined 9-mer core PYPQQQQPY (SEQ ID NO:8) associated with CeD has been identified, together with longer CeD associated peptides containing this epitope.

Gut proteolysis of wheat by gastric and pancreatic enzymes is heterogenous, and it has become well established that predicted enzyme specificity is only partially reflecting the actual variation in digest. Thus, to get a more realistic picture of what is a likely protein fragmentation pattern, we manufactured the A0A0E3SZN6_TRIUA, as well as the main wheat ω-gliadin Q9FUW7_WHEAT and α-gliadin Q9M4L6_WHEAT proteins (FIG. 9A to C) and performed an in vitro chymotrypsin digest, essentially as described (Arentz-Hansen E H, et ai. Gut 2000:446-51 and Molberg O, et al., Methods Mol Med. 2000; 41:105-24). These proteolytic digests were then analyzed by mass spectrometry, essentially as described (Dorum et al., 2014, supra), FIG. 9D-F.

Indeed, the α-gliadin control protein Q9M4L6_WHEAT was fragmented into a variety of smaller peptide fragments (FIG. 9D), including the well-characterized 33-mer multi-epitope segment (Shan L, et al., Science. 2002, 297(5590):2275-9.). The extensive proteolysis observed is well in line with previous reports (Dorum et al., 2014, supra). Addressing the same proteolysis of A0A0E3SZN6_TRIUA, this protein was also fragmented into a variety of smaller peptides (FIG. 9E). Importantly, no destructive processing was observed of the postulated novel 9-mer core peptide and multiple species seen would be expected to have HLA-DQ2.5 binding capacity in line with what was already seen in the previous analyses (FIG. 8). The minimum proteolytic segment identified from A0A0E3SZN6_TRIUA corresponded to the postulated A0A0E3SZN6_p4Q_p6Q_medium 13-mer peptide (FIGS. 8C and 9E). As Triticum urartu is the diploid A genome ancestor of the modern hexaploidy Triticum aestivum wheat crop (Marcussen et al., 2014, Science:345 (6194), 1250092), it is highly likely that the A0A0E3SZN6_TRIUA encoding gene is found as part of the highly complex domesticated crop genome and thereby represent a relevant food source for human consumption (Appels et al., 2018, Science 361 (6403), eaar7191 and Juhasz et al., 2018, Science Advances 4(8), eaar 8602). Indeed, signature motifs from food source analysis have pointed to A0A0E3SZN6 as such a component (Spada et al., 2020, Front. Nutr. 7:98). Correspondingly, we see that the major known (ω-gliadin cannot be the genetic source of the epitope as it only encodes eight of nine necessary residues to build the 9-mer core binding to the HLA (FIG. 9F).

We noted also that many of the peptides in the A0A0E3SZN6_TRIUA MS analysis (FIG. 9E) lacked the p10Gly residue that appeared imperative for efficient 4.7C binding to the DQ2.5-glia-α1a epitope (FIG. 7). Thus, these data strongly indicated that these two peptides take different shapes in the HLA groove and also are decoded by the 4.7C antibody in a different manner. This was subsequently confirmed by alanine scanning experiments.

A large body of experimental evidence points to two main mechanisms on how the human immune system acquires the ability to present pathogenic gluten peptides in CeD (Lindfors, K., et al., 2019, Nat Rev Dis Primers 5, 3). These observations corroborate the central role B cells appear to have in this disease, which chiefly falls into two categories, namely tissue transglutaminase 2 (TG2) and gliadin specific B cells. With respect to the latter, an extensive characterization of the B cell receptor (BCR) of these gliadin peptide specific B cells have identified the particularly frequent sequence motif (QPQQPFP (SEQ ID NO:64)) that is part of these peptides that most often is of the omega gliadin type (Dorum S., et al., 2016, Sci Rep. 6:25565) and which forms an N-terminal extension of CD4⁺ T cell epitope. Based on the in silico (FIG. 8) and MS analyses (FIG. 9) of both the T. aestivum Q9M4L6_WHEAT and T. urartu A0A0E3SZN6_TRIUA proteins, we noted that in contrast to the α-gliadin sequence of Q9M4L6_WHEAT, which lacks this BCR consensus sequence, the ω-gliadin sequence of A0A0E3SZN6_TRIUA has a closely related QPQQPYP (SEQ ID NO:37) motif in its N-terminus, with the Q/E position of the BCR epitope (which is overlapping with the T cell epitope) corresponding to position p-2 when annotated according to the HLA 9-mer core. To investigate if the A0A0E3SZN6_TRIUA epitope had the capacity to serve as target for such BCRs, we therefore reconstructed two CeD patient derived prototypic anti-gliadin peptide BCRs (termed 1002-1E01 and 1002-1E03) as soluble antibodies and tested them for binding to Neutravidin-immobilized peptide in ELISA (FIG. 10), essentially as described (Snir et al., 2017, JCI Insight, 2(16):e93961).

The two BCRs chosen have been extensively described and differ in their binding profile to the target sequence (FIG. 10A). Importantly, a critical feature of the known QPQQPFP (SEQ ID NO:64) motif is deamidation modification by TG2, into QPEQPFP (SEQ ID NO:65) which is a signature feature separating healthy individuals from those with CeD. The two BCRs differ in their requirement for this TG2 modification, and to validate our approach, we therefore included two positive control peptides with either Gln (Q) or Glu (E) in this position (PC1 and PC2, FIG. 10B). The results indeed showed strong reactivity of the BCRs to the appropriate PCs (FIG. 10C), and the 1002-1E03 antibody showed an apparent equally good binding to the A0A0E3SZN6_TRIUA epitope, but only with a Glu (E) in p-2. The 1002-1E01 antibody only reacted with the PC peptides, which is likely explained by its documented strong requirement for Phe (F) in position p2. Thus, our results indeed confirmed that the A0A0E3SZN6_TRIUA T cell epitope harbors an N-terminal QPEQPYP (SEQ ID NO:38) extension that serves as an excellent target for a TG2-sensitive prototypic gliadin peptide-specific BCR that recurringly is shared across CeD patients (Snir et al., 2017, supra). Given that there is a difference in the BCR epitope described in comparison with the one seen in the A0A0E3SZN6_TRIUA sequence, where the Phe (F) residue is exchanged with a Tyr (Y) in the latter, we wondered if the 1002-1E03 antibody had a preference for either sequence. Thus, we repeated the experiment in a serial dilution of the antibody to estimate the individual EC50. Indeed, the results showed that both sequences were equally well recognized (FIG. 10D).

In summary, these results thus point to a well-documented mechanism by which the A0A0E3SZN6_TRIUA BCR epitope may lead to generation of strong antibody responses by effective presentation of the T cell epitope and thus establishment of T-cell help to B cells. This finding further reinforces the relevance of our patient staining data by use of the 107 and 4.7C antibodies (FIGS. 1 and 5) which identifies B cells and their plasma cell (PC) offspring as the dominating gluten peptide presenting cells in the inflamed gut of CeD patients. That B cells play an imperative role in the GI tissue destruction characteristic of CeD has further gained strong evidence in the only murine CeD model that recapitulates the villus atrophy seen in CeD, where B cell depletion completely abrogated gluten induced atrophy. In summary, our data fully recapitulates major critical components necessary to fulfil the requirements for being a pathogenic gluten epitope in CeD and pointing to the A0A0E3SZN6_TRIUA sequence protein as the source in food.

Production of Recombinant Soluble pHLA Molecules

Recombinant soluble pHLA (rs-pHLA) is a valuable reagent in immunological research, but in contrast to HLA class I, these reagents are generally still very difficult to manufacture for HLA class II, and HLA-DQ has proven particularly difficult in this manner partly due to unknown reasons (Davis et al., 2011, Nat. Rev. Imm., 11, 551-558). In other reports, it is well documented that in nature HLA-DQ2.5 has a very narrow peptide repertoire with a distinct phenotype pointing to special requirements to the peptides for being able to productively be combined with the HLA (Fallang et al., 2009, Nat. Imm., 10, 1096-1101 and Bergseng, E., et al, 2015, Immunogenetics 67, 73-84). To further validate the A0A0E3SZN6_TRIUA T cell epitope candidate, we sought to generate rs-pHLA complexes essentially as previously described (Quarsten et al J Immunol, 2001, 167: 4861). To benchmark the performance, we included the two already reported versions harboring the DQ2.5-glia-α1a and DQ2.5-glia-α2 T cell epitopes, respectively.

Indeed, it was possible to produce all three rs-pHLA complexes by this method, which comprises covalent coupling of the T cell epitope to the N-terminus of the HLA β-chain by use of a synthetic linker and expression concomitantly with engineered in vivo biotinylation in Sf9 insect cells. The yield and purity of the affinity purified (FLAG-tag purification) material from these expression cultures varied between each peptide variant as expected but overall, they were in the usual range seen for such molecules (FIG. 11D). Thus, the sheer ability to manufacture these molecules to fairly good homogeneity (FIG. 11A-C), including the pHLA containing a T cell epitope of the invention (DQ2.5-glia-NTP-001), is strongly indicative of being true T cell epitopes in the context of HLA-DQ2.5.

To further assess the integrity of these rs-pHLA complexes, we conducted neutravidin capture ELISA binding experiments, essentially as described (Frick R., et al., 2021, Sci. Immunol. 6(62):eabg4925) to panels of selected well-documented pan-HLA and TCR-Like antibodies. All three versions showed a good and concentration dependent binding to the conformation specific pan-DQ antibody SPV-L3, whereas no such binding was seen with the conformation specific pan-DR antibody L243 (FIG. 12A-C), strongly indicative of correctly folded molecules. The two TCR-Like antibodies 107 and 4.7C were developed by use of rs-pHLA of HLA-DQ2.5: DQ2.5-glia-α1a highly similar to the versions produced here and when tested for binding to this complex in the ELISA, we indeed showed highly specific, and concentration dependent binding as expected (FIG. 12D), and which parallels previous experiments (FIGS. 1A and 2B), and also with a binding hierarchy where the high affinity 4.7C was superior to low affinity mother clone 107. This binding profile was closely mirrored when repeated with the HLA-DQ2.5: DQ2.5-glia-NTP-001 (FIG. 12E). In contrast, no binding to these complexes were seen with the 3.C11 antibody that specifically bound only to its HLA-DQ2.5: DQ2.5-glia-α2 target (FIG. 12F). Collectively, these results show that all three HLA complexes are correctly folded and binding only to strictly conformation dependent ligands.

We thus conclude that indeed the DQ2.5-glia-NTP-001 (A0A0E3SZN6_p-2E_p6E_medium) epitope candidate fulfils the strict requirements for being successfully produced as a fully functional intact rs-pHLAII in the context of HLA-2.5 on par with the two other known immunodominant CeD epitopes DQ2.5-glia-α1a and DQ2.5-glia-α2.

### T Cell Activation

Through the line of experiments described above, the data clearly show that the novel DQ2.5-glia-NTP-001 peptide fulfils a strict set of requirements to both be the source of the observed TCR-Like antibody reactivity in the CeD patient material (FIG. 5), as well as doing this by virtue of containing an HLA-DQ2.5 restricted CeD-specific T cell epitope that also cross-talks with the anti-DGP BCRs well documented as part of the CeD pathogenesis. We have as of yet not identified defined T cell clones (TCC) from CeD tissue, which would allow us to, in a highly controlled manner, assess the T cell reactivity potential. It is however well documented that quite a few CeD TCCs are somewhat promiscuous and readily cross-react with multiple gluten epitopes (e.g. Dahal-Koirala, S., et al., 2016, supra). From past studies we do have a panel of such TCCs as SKW-3 reconstructions (Frick et al., 2021, supra, and Petersen et al., 2014, supra). We therefore chose to test different versions of the A0A0E3SZN6 peptide in T cell activation assays against such SKW-3 cells. We included the two known DQ2.5-glia-α1a reactive clones 380 and S2, as well as the DQ2.5-glia-α2 reactive 364. Notably, the 380 clone is known to not distinguish between the two closely related epitopes DQ2.5-glia-α1a and DQ2.5-glia-ω1 (Gunnarsen et al., 2017, supra). We first estimated the peptide sensitivity and specificity between S2 and 380 towards DQ2.5-glia-α1a, and both were confirmed specific and the S2 clone being somewhat more sensitive (FIG. 13A).

We then repeated the assay, but now with a panel of the various A0A0E3SZN6 versions, as outlined in FIG. 8C. None of these peptides showed any reactivity, whereas all control peptides readily activated the T cells (FIG. 13B). Collectively, this again underpins the notion that the A0A0E3SZN6 derived peptide represents a novel T cell epitope.

The prevalence of CeD specific T cells varies between different patients and within each patient on a spatial (tissue versus peripheral) and temporal (time) gradient. However, it is well documented that even at high inflammatory status the absolute frequency is low (Risnes et al., J Clin Invest. 2018; 128(6):2642-2650). With a tissue abundance of up to 1-2% at the most, between 30-50% of these T cells have an unknown reactivity (Raki et al., 2017, supra, and Qiao S-W, et al, 2021, Front. Immunol. 12:646163). Further, some of these cells migrate at a low frequency between blood and the gut, and we thus chose to use a very sensitive intracellular IFNγ cytokine flow assay to assess whether we could detect an apparent CeD specific reactivity to the A0A0E3SZN6-derived candidate using HLA-DQ2.5 typed peripheral blood mononuclear cells (PBMCs) from either confirmed CeD patients or healthy controls (HC) (FIG. 14).

The total amount of T cells in PBMCs varies between 45-75%. Thus, we first established the baseline IFN-γ detection levels in CeD PBMC T cells (CD3+/CD4+) in absence of any exogenous peptide (FIG. 14A). We then used cultivated CeD and HC PBMCs from an HLA-DQ2.5 positive CeD (donor 595) and a HC (donor 557) in the presence of either the well-characterized deamidated 33-mer peptide from T. aestivum α-gliadin, or the deamidated long version of the T. urartu A0A0E3SZN6 ω-gliadin for 48h (FIG. 14B). Indeed, here we did see a clear expansion of IFN-γ positive CD4 T cells towards both peptides in the CeD material whereas no such expansion was seen in the HC (FIG. 14B). The apparent expansion was slightly higher for the 33-mer as compared to the A0A0E3SZN6 peptide (average 4.2% versus 2.8%). Notably, this apparent expansion of INF-γ positive cells was not seen in the CD3+/CD4− cell population (FIG. 14C).

To get a better understanding of individual variation in responses, we repeated the experiment with an extended number of PBMC samples covering three different CeD donors, as well as two HCs (FIG. 15). This is important, as it is well known that T cell responses and amplitude against gluten epitopes vary between CeD individuals, and responses in blood are usually more variable due to low T cell abundance as compared with gut biopsy material. Firstly, we confirmed the peptide-specific responses in CeD donor 595, and despite the frequently seen variation in absolute responses in such assays, the peptide reactivity hierarchy remained essentially identical (FIG. 15A). Moreover, we indeed here also observed a CeD donor dependent peptide reactivity variation in that donor 600 appeared to favor the A0A0E3SZN6 peptide, donor 595 was about equal on both (no statistical difference), and donor 585 appeared to favor the known 33-mer from T. aestivum α-gliadin (FIG. 15A). Hence, these two epitopes both exhibited donor-specific responses and with a presence in 2/3 each in the tested PBMC material. Importantly, no responses were seen in the two HCs (FIG. 15B). In summary, these data strongly suggests that the T. urartu A0A0E3SZN6 sequence is a true T cell epitope in the context of HLA-DQ2.5 which appears restricted to CeD on par with the 33-mer peptide from T. aestivum α-gliadin.

1. A peptide comprising an epitope that comprises the amino acid sequence PYPQQQQPY (SEQ ID NO:8) or an epitope that comprises the amino acid sequence PYPQQQQPY (SEQ ID NO:8) in which one or more of the Q residues is replaced by an E residue, wherein the peptide is up to 40 amino acids in length.

2. The peptide of claim 1, wherein said peptide comprises the amino acid sequence of QPQQPYPQQQQPY (SEQ ID NO:20), PQQPYPQQQQPYGT (SEQ ID NO:24), or QPQQPYPQQQQPYGTSL (SEQ ID NO:22), or an amino acid sequence wherein one or more of said Q residues is replaced by an E residue,
or an amino acid sequence substantially homologous thereto wherein said substantially homologous sequence comprises a sequence with 1, 2 or 3 amino acid substitutions, additions or deletions, and wherein said amino acid substitutions, additions or deletions are located outside of the PYPQQQQPY core sequence as defined in claim 1.

3. The peptide of claim 1 or claim 2 wherein said epitope comprises the amino acid sequence PYPQQEQPY (SEQ ID NO:25), PYPEQEQPY (SEQ ID NO:26), or PYPEQQQPY (SEQ ID NO:27).

4. The peptide of any one of claims 1 to 3, wherein said peptide comprises a sequence obtainable by transglutaminase-2 (TG2) deamidating said amino acid sequence.

5. The peptide of any one of claims 1 to 4, wherein said peptide comprises the amino acid sequence PYPQQEQPY (SEQ ID NO:25), preferably wherein said peptide comprises QPQQPYPQQEQPY (SEQ ID NO:28), QPQQPYPQQEQPYGTSL (SEQ ID NO:30), or PQQPYPQQEQPYGT (SEQ ID NO:29).

6. The peptide of any one of claims 1 to 5, wherein said peptide comprises the amino acid sequence QPQQPYPQQQQPY (SEQ ID NO:20) and wherein said Q at residue 3 is replaced by an E residue, and/or wherein said Q at residue 10 is replaced by an E residue, and/or wherein said Q at residue 8 is replaced by an E residue; or
wherein said peptide comprises the amino acid sequence PQQPYPQQQQPYGT (SEQ ID NO:24) and wherein said Q at residue 2 is replaced by an E residue, and/or
wherein said Q at residue 9 is replaced by an E residue, and/or wherein said Q at residue 7 is replaced by an E residue; or
wherein said peptide comprises the amino acid sequence QPQQPYPQQQQPYGTSL (SEQ ID NO:22) and wherein said Q at residue 3 is replaced by an E residue, and/or wherein said Q at residue 10 is replaced by an E residue, and/or wherein said Q at residue 8 is replaced by an E residue.

7. The peptide of any one of claims 1 to 6, wherein said peptide comprises the amino acid sequence:
(SEQ ID NO: 28)
QPQQPYPQQEQPY,
(SEQ ID NO: 31)
QPEQPYPQQEQPY,
(SEQ ID NO: 32)
QPEQPYPQQQQPY,
(SEQ ID NO: 30)
QPQQPYPQQEQPYGTSL,
(SEQ ID NO: 33)
QPEQPYPQQEQPYGTSL,
(SEQ ID NO: 34)
QPEQPYPQQQQPYGTSL,
(SEQ ID NO: 29)
PQQPYPQQEQPYGT,
(SEQ ID NO: 35)
PEQPYPQQEQPYGT,
or
(SEQ ID NO: 36)
PEQPYPQQQQPYGT.

8. The peptide of any one of claims 1 to 7, wherein said peptide comprises the amino acid sequence QPQQPYP (SEQ ID NO:37) or QPEQPYP (SEQ ID NO:38).

9. The peptide of claim 8, wherein said peptide comprises the amino acid sequence QPEQPYPQQEQPY (SEQ ID NO:31) or QPEQPYPQQEQPYGTSL (SEQ ID NO:33).

10. The peptide of any one of claims 1 to 9, wherein said peptide comprises a G residue at position 10 or at the position corresponding to position 10, preferably said peptide comprises the residues G and T at positions 10 and 11 or at the positions corresponding to positions 10 and 11, or the residues G and L at positions 10 and 13 or at the positions corresponding to positions 10 and 13, wherein said positions are defined in relation to the 9-mer as defined in claim 1.

11. A conjugate or complex comprising a peptide as defined in any one of claims 1 to 10 coupled to or associated with an MHC molecule.

12. One or more nucleic acid molecules comprising nucleotide sequences that encode a peptide as defined in any one of claims 1 to 10 or a conjugate or complex as defined in claim 11.

13. An expression vector comprising the nucleic acid molecules of claim 12, or a cell comprising said expression vector or comprising the nucleic acid molecules of claim 12.

14. A composition comprising a peptide as defined in any one of claims 1 to 10, a conjugate or complex as defined in claim 11, the nucleic acid molecules as defined in claim 12, or the expression vectors or cells as defined in claim 13.

15. The composition of claim 14, wherein said composition is a vaccine composition.

16. A method for diagnosing celiac disease in a subject, said method comprising contacting a sample from said subject with a peptide as defined in any one of claims 1 to 10 or a conjugate or complex as defined in claim 11, and determining whether said peptide, conjugate or complex binds to T cells in said sample, or whether said sample contains antibodies which bind to said peptide, conjugate or complex, wherein the binding of said peptide, conjugate or complex to T cells, or the presence of said antibodies in the sample, indicates that the subject has, or is susceptible to, celiac disease.

17. A peptide as defined in any one of claims 1 to 10, or a conjugate or complex as defined in claim 11, or nucleic acid molecules as defined in claim 12, or expression vectors or cells as defined in claim 13, for use in therapy, preferably for use in the treatment or prevention of celiac disease, or for use in the tolerization of a subject to said peptide, conjugate or complex, or for use to suppress or reduce an immune response to said peptide, conjugate or complex.

18. The use of a peptide as defined in any one of claims 1 to 10, or a conjugate or complex as defined in claim 11, or nucleic acid molecules as defined in claim 12, or expression vectors or cells as defined in claim 13, in the manufacture of a medicament or composition for use in therapy, preferably for use in the treatment or prevention of celiac disease, or for use in the tolerization of a subject to said peptide, conjugate or complex, or for use to suppress or reduce an immune response to said peptide, conjugate or complex.

19. A method of treating or preventing CeD in a subject, or a method of tolerization of a subject, or a method of suppressing an immune response in a subject, said method comprising the step of administrating an effective amount of a peptide as defined in any one of claims 1 to 10, or a conjugate or complex as defined in claim 11, or nucleic acid molecules as defined in claim 12, or expression vectors or cells as defined in claim 13, to said subject.

20. A binding protein that specifically binds to a conjugate or complex as defined in claim 11.

21. The binding protein of claim 20, wherein said binding protein comprises a T cell receptor, or an antibody, or an antigen-binding domain of an antibody.

22. The binding protein of claim 21, wherein said T cell receptor is a soluble T cell receptor.

23. The binding protein of claim 20 or claim 21, wherein said binding protein is expressed on the surface of cells, preferably eukaryotic cells, more preferably T cells or NK cells.

24. The binding protein of any one of claims 20 to 22, wherein said binding protein is associated with a payload such as a cytotoxic moiety or siRNA, or is associated with a second binding protein with specificity for effector cells.

25. The binding protein of any one of claims 20 to 24 for use in therapy, preferably for use in the treatment or prevention of celiac disease.

26. The use of the binding protein of any one of claims 20 to 24, in the manufacture of a medicament or composition for use in therapy, preferably for use in the treatment or prevention of celiac disease.

27. A method of treating or preventing celiac disease in a subject, said method comprising the step of administrating an effective amount of a binding protein of any one of claims 20 to 24 to said subject.

28. A method of producing the binding protein of any one of claims 20 to 22, said method comprising the use of a conjugate or complex as defined in claim 11.
